SEQUENCE LISTING (48 sequences)

SEQ ID NO: 1 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 2 - protein, 20 aa, Homo sapiens
MSRSVALAVLALLSLSGLEA

SEQ ID NO: 3 - protein, 99 aa, Homo sapiens [sequence omitted]

SEQ ID NO: 4 - protein, 297 aa, synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 5 - protein, 296 aa, synthetic construct
AEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 6 - protein, 5 aa, synthetic construct
GGGGS

SEQ ID NO: 7 - protein, 15 aa, synthetic construct
GGGGSGGGGSGGGGS

SEQ ID NO: 8 - protein, 20 aa, synthetic construct
GGGGSGGGGSGGGGSGGGGS

SEQ ID NO: 9 - protein, 341 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 10 - protein, 430 aa, synthetic construct
MSRSVALAVLALLSLSGLEAAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 11 - protein, 410 aa, synthetic construct
AEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 12 - protein, 791 aa, synthetic construct
MSRSVALAVLALLSLSGLEAAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 13 - protein, 771 aa, synthetic construct
AEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 14 - protein, 9 aa, synthetic construct
SLLMWITQC

SEQ ID NO: 15 - protein, 9 aa, synthetic construct
RMFPNAPYL

SEQ ID NO: 16 - protein, 10 aa, synthetic construct
ELAGIGILTV

SEQ ID NO: 17 - protein, 9 aa, synthetic construct
NLVPMVATV

SEQ ID NO: 18 - protein, 83 aa, synthetic construct
CGGAGATGTGTATAAGAGACAGNNNNNNNNNNGGACGCAACTTAAGANNNNNNNNNCCCATATAAGAAAWHEREINNACGORT

SEQ ID NO: 19 - protein, 206 aa, Staphylococcus aureus
MKKWTNRLMTIAGVVLILVAAYLFAKPHIDNYLHDKDKDEKIEQYDKNVKEQASKDKKQQAKPQIPKDKSKVAGYIEIPDADIKEPVYPGPATSEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVK

SEQ ID NO: 20 - protein, 147 aa, Staphylococcus aureus
QAKPQIPKDKSKVAGYIEIPDADIKEPVYPGPATSEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWEKRKIFVATEVK

SEQ ID NO: 21 - protein, 206 aa, Staphylococcus aureus
MKKWTNRLMTIAGVVLILVAAYLFAKPHIDNYLHDKDKDEKIEQYDKNVKEQASKDKKQQAKPQIPKDKSKVAGYIEIPDADIKEPVYPGPATSEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWETRKIFVATEVK

SEQ ID NO: 22 - protein, 147 aa, Staphylococcus aureus
QAKPQIPKDKSKVAGYIEIPDADIKEPVYPGPATSEQLNRGVSFAEENESLDDQNISIAGHTFIDRPNYQFTNLKAAKKGSMVYFKVGNETRKYKMTSIRDVKPTDVGVLDEQKGKDKQLTLITCDDYNEKTGVWETRKIFVATEVK

SEQ ID NO: 23 - protein, 249 aa, Streptococcus pyogenes
MVKKQKRRKIKSMSWARKLLIAVLLILGLALLFNKSIRNTLIARNSNKYQVTKVSKKQIKKNKEAKSTFDFQAVEPVSTESVLQAQMAAQQLPVIGGIAIPELGINLPIFKGLGNTELIYGAGTMKEEQVMGGENNYSLASHHIFGITGSSQMLFSPLERAQNGMSIYLTDKEKIYEYIIKDVFTVAPERVDVIDDTAGLKEVTLVTCTDIEATERIIVKGELKTEYDFDKAPADVLKAFNHSYNQVST

SEQ ID NO: 24 - protein, 168 aa, Streptococcus pyogenes
VLQAQMAAQQLPVIGGIAIPELGINLPIFKGLGNTELIYGAGTMKEEQVMGGENNYSLASHHIFGITGSSQMLFSPLERAQNGMSIYLTDKEKIYEYIIKDVFTVAPERVDVIDDTAGLKEVTLVTCTDIEATERIIVKGELKTEYDFDKAPADVLKAFNHSYNQVST

SEQ ID NO: 25 - protein, 41 aa, synthetic construct
LXXTXWHEREINXPAORSXANYAMINOACIDANDXGASORC

SEQ ID NO: 26 - protein, 25 aa, synthetic construct
LPXTGWHEREINXANYAMINOACID

SEQ ID NO: 27 - protein, 25 aa, synthetic construct
LPXTAWHEREINXANYAMINOACID

SEQ ID NO: 28 - protein, 25 aa, synthetic construct
LPXTSWHEREINXANYAMINOACID

SEQ ID NO: 29 - protein, 451 aa, synthetic construct
VTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 392 aa, synthetic construct
QVTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSGGGGSGGGGSQAKPQIPKDKSKVAGYIEIPDADIKEPVYPGPATSEQLNRGVSFAEENESLDDQNISIAGHTFIDRPNYQFTNLKAAKKGSMVYFKVGNETRKYKMTSIRNVKPTDVGVLDEQKGKDKQLTLITCDDYNEKTGVWETRKIFVATEVK

SEQ ID NO: 31 - protein, 219 aa, synthetic construct
SIQMTQSPSSLSASVGDRVTITCQSSHSVYSDNDLAWYQQKPGKAPKLLIYQASKLASGVPSRFSGSGSGTDFTLTISSLQPEDFATYYCLGGYDDESDTYGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 32 - protein, 219 aa, synthetic construct
DIQMTQSPSSLSASVGDRVTITCQSSHSVYSDNDLAWYQQKPGKAPKLLIYQASKLASGVPSRFSGSGSGTDFTLTISSLQPEDFATYYCLGGYDDESDTYGFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 121 aa, synthetic construct
VTLKESGPVLVKPTETLTLTCTVSGFSLSTYSMSWIRQPPGKALEWLGFIGSRGDTYYASWAKGRLTISKDTSKSQVVLTMTNMDPVDTATYYCARERDPYGGGAYPPHLWGRGTLVTVSS

SEQ ID NO: 34 - protein, 10 aa, synthetic construct
GFSLSTYSMS

SEQ ID NO: 35 - protein, 16 aa, synthetic construct
FIGSRGDTYYASWAKG

SEQ ID NO: 36 - protein, 14 aa, synthetic construct
ERDPYGGGAYPPHL

SEQ ID NO: 37 - protein, 24 aa, synthetic construct
VTLKESGPVLVKPTETLTLTCTVS

SEQ ID NO: 38 - protein, 14 aa, synthetic construct
WIRQPPGKALEWLG

SEQ ID NO: 39 - protein, 32 aa, synthetic construct
RLTISKDTSKSQVVLTMTNMDPVDTATYYCAR

SEQ ID NO: 40 - protein, 9 aa, synthetic construct
RGTLVTVSS

SEQ ID NO: 41 - protein, 111 aa, synthetic construct
IQMTQSPSSLSASVGDRVTITCQSSHSVYSDNDLAWYQQKPGKAPKLLIYQASKLASGVPSRFSGSGSGTDFTLTISSLQPEDFATYYCLGGYDDESDTYGFGGGTKVEIK

SEQ ID NO: 42 - protein, 13 aa, synthetic construct
QSSHSVYSDNDLA

SEQ ID NO: 43 - protein, 7 aa, synthetic construct
QASKLAS

SEQ ID NO: 44 - protein, 12 aa, synthetic construct
LGGYDDESDTYG

SEQ ID NO: 45 - protein, 22 aa, synthetic construct
IQMTQSPSSLSASVGDRVTITC

SEQ ID NO: 46 - protein, 15 aa, synthetic construct
WYQQKPGKAPKLLIY

SEQ ID NO: 47 - protein, 32 aa, synthetic construct
GVPSRFSGSGSGTDFTLTISSLQPEDFATYYC

SEQ ID NO: 48 - protein, 10 aa, synthetic construct
FGGGTKVEIK

(19) United States
(12) Patent Application Publication
(10) Pub. No.: US 2025/0347693 A1
(43) Pub. Date: Nov. 13, 2025

(54) T Cell Receptor Identification

(71) Applicant: Hoffmann-La Roche Inc., Little Falls, NJ (US)

(72) Inventors: Stephan Gasser, Fislisbach (CH); Florian Kast, Zurich (CH); Pablo Umaña, Wollerau (CH); Dario Venetz, Zurich (CH)

(73) Assignees: Hoffmann-La Roche Inc., Little Falls, NJ (US); Hoffmann-La Roche Inc., Little Falls, NJ (US)

(21) Appl. No.: 19/073,394

(22) Filed: Mar. 7, 2025

Related U.S. Application Data
(63) Continuation of application No. PCT/EP2023/074374, filed on Sep. 6, 2023.

(30) Foreign Application Priority Data
Sep. 8, 2022 (EP) 22194490.3
Feb. 15, 2023 (EP) 23156673.8

(51) Int. Cl. G01N 33/569; C07K 14/74; C07K 19/00; C12N 9/14; C12N 15/85; G01N 33/68

(52) U.S. Cl. CPC G01N 33/56972; C07K 14/70539; C07K 19/00; C12N 9/14; C12N 15/85; G01N 33/6872; C07K 2319/61; C12Y 308/01005; G01N 2333/7051; G01N 2333/70539; G01N 2333/914

(57) ABSTRACT

The present disclosure relates to the fields of molecular biology and immunology.

### CROSS-REFERENCE TO RELATED APPLICATIONS

This application is a continuation of PCT Application No. PCT/Eβ2023/074374. filed September 6. 2023, which claims benefit of priority under 35 U.S.C. § 119 to European Patent Application No 22194490.3, filed Sep. 8, 2022, and European Patent Application No. 23156673.8. filed Feb. 15, 2023. each of which is incorporated by reference in its entirety.

### SEQUENCE LISTING

This application contains a Sequence Listing which has been submitted electronically in XML format and is hereby incorporated bs reference in its entirety. Said XML copy. created on Jan. 16, 2025. is named P37751 sequence listing.xml and is 48.832 bytes in size.

### TECHNICAL FIELD

The present disclosure relates to the fields of molecular biology and immunology.

### BACKGROUND

Adoptive T cell therapy (ACT) is a powerful approach to the treatment of cancer, using cancer-specific T cells (Rosenberg and Restifo. Science (2015) 348(6230).62-68). The cells employed in ACT are typically either naturally-occurring cancer antigen-specific cells, or T cells engineered to express a TCR specific for cells expressing a MHC:peptide complexes comprising a peptide of a target antigen of interest (i.e. TCR-engneered T cells), or T cells engineered to express chimeric antigen receptors (CARs) comprising an antigen-binding domain specific for the target antigen of interest (CAR-engineered T cells: Rosenberg and Restifo. Science (2015) 348(6230):62-68). Engineering such cells requires the identification of the appropriate target antigens and targeting molecules (Leko and Rosenberg. Cancer Cell (2020) 38(4):454-472). TCR discovery remains a challenging endeavor (Joglekar and ii, Nat Methods (2021) 18(8):873-880), Rapid and precise identification of neoantigens and evaluation of their immunogenicity is important for widespread adoption of ACT (Garcia-Garijo el al., Front Immunol. (2019) 10:1392).

Trogocytosis is a well-established process that occurs bidirectionally between T cells and cells expressing MHC:peptide complexes recognised by the TCRs they express, during immune interactions (Miyake and Karasuyama. Cells (2021) 10(5):1255). Identification of HLA:peptide targets of orphan TCRs has been achieved by evaluation of trogocytosis of membrane contents from the T cell onto the HLA:peptide-presenting target cell (Li et al, Nat Methods (2019) 16(2):183-190).

There remains a need in the art for techniques providing for high-throughput identification of TCR and MHC:peptide interaction partners.

### SUMMARY

In a first aspect. the present disclosure provides a polypeptide comprising (i) the amino acid sequence of a major histocompatibility complex (MHC) polypeptide, and (ii) a moiety facilitating labelling of the polypeptide with an identifier moiety.

In some embodiments in accordance with the various different aspects described herein relating to polypeptides. the MHC polypeptide is β2 microglobulin.

In some embodiments in accordance with the various different aspects described herein relating to polypeptides, the identifier moiety is a nucleic acid moiety. In some embodiments, the identifier moiety comprises or consists of single-stranded DNA (ssDNA).

In some embodiments in accordance with the various different aspects described herein relating to polypeptides, the moiety facilitating labelling of the polypeptide with an identifier moiety is or comprises a self-labelling protein tag In some embodiments, the moiety facilitating labelling of the polypeptide with an identifier moiety is or comprises a HaloTag®.

In some embodiments in accordance with the various different aspects described herein relating to polypeptides, the polypeptide further comprises a sortase substrate motif.

In some embodiments in accordance with the various different aspects described herein relating to polypeptides. the polypeptide further comprising a detectable moiety.

In some embodiments in accordance with the various different aspects described herein relating to polypeptides, the detectable moiety is a fluorescent label.

In some embodiments in accordance with the various different aspects described herein relating to polypeptides, the polypeptide comprises or consists of an amino acid sequence having at least 70% amino acid sequence identity to SEQ ID NO. 11, 10, 13 or 12.

The present disclosure also provides a polypeptide comprising (i) the amino acid sequence of β2 microglobulin, and (ii) Halolag®.

The present disclosure also provides a polypeptide comprising (i) the amino acid sequence of β2 microglobulin. (ii) a sortase substrate motif, and (iii) HaloTag.

The present disclosure also provides a MHC molecule comprising a polypeptide according to the present disclosure. 10 The present disclosure also provides a MHC:peptide complex, comprising a MHC molecule according to the present disclosure, and a peptide presented by the MHC molecule.

The present disclosure also provides a nucleic acid. or a plurality of nucleic acids, encoding a polypeptide according to the present disclosure.

In some embodiments in accordance with the various different aspects described herein relating to nucleic acids, the nucleic acid or plurality of nucleic acids further comprises nucleic acid encoding a peptide presented by a MHC molecule comprising a polypeptide according to the present disclosure.

The present disclosure also provides an expression vector. or a plurality of expression vectors, comprising a nucleic acid. or a plurality of nucleic acids, according to the present disclosure.

The present disclosure also provides a cell comprising a polypeptide, MHC molecule. MHC:peptide complex, nucleic acid or plurality of nucleic acids, or expression vector or plurality of expression vectors according to the present disclosure.

In some embodiments in accordance with the various different aspects described herein relating to cells, the cell is an antigen presenting cell (APC).

The present disclosure also provides a method for producing a cell comprising a NIC molecule labelled with an identifier moiety, comprising
(1) introducing into a cell a nucleic acid or plurality of nucleic acids according to the present disclosure; and
(2) contacting the cell with a labelling moiety comprising an identifier moiety. wherein the labelling moiety is suitable for labelling the polypeptide encoded by the nucleic acid or plurality of nucleic acids of (1) with an identifier moiety.

The present disclosure also provides a method for producing a cell comprising a MHC:peptide complex comprising a MHC molecule labelled with a single-stranded DNA (ssDNA) moiety, comprising:
(1) introducing into a cell a nucleic acid or plurality of nucleic acids encoding a polypeptide comprising (i) the amino acid sequence of β2 microglobulin, and (ii) a HaloTag,:
(2) introducing into the cell: (i) a peptide presented by a MHC molecule comprising the polypeptide of (1). or (ii) nucleic acid encoding a peptide presented by a MHC molecule comprising the polypeptide of (1); and
(3) contacting the cell with a HaloTag® ligand comprising the ssDNA moiety and a chloroalkane moiety.

The present disclosure also provides a method for producing a cell comprising a MHC:peptide complex comprising a MHC molecule labelled with a single-stranded DNA (ssDNA) moiety. comprising:
(1) introducing into a cell a nucleic acid or plurality of nucleic acids encoding a 20 polypeptide comprising (i) the amino acid sequence of β2 microglobulin., (ii) a sortase substrate motif, and (iii) a HaloTag®:
(2) introducing into the cell: (i) a peptide presented by a MHC molecule comprising the polypeptide of (1). or (ii) nucleic acid encoding a peptide presented by a MHC molecule comprising the polypeptide of (1); and
(3) contacting the cell with a HaloTag® ligand comprising the ssDNA moiety and a chloroalkane moiety.

The present disclosure also provides a cell produced by a method for producing a cell comprising a MHC:peptide complex according to the present disclosure.

The present disclosure also provides a composition comprising a cell according to the present disclosure, and a T cell.

The present disclosure also provides a method for identifying a T cell receptor (TCR) that binds to an MHC:peptide complex, comprising:
(1) contacting a cell comprising an MHC:peptide complex according to claim 13 with a population of T cells:
(2) incubating the cells under conditions suitable for trogocytosis of an MHC:peptide complex by a T cell comprising a TCR that binds to the MHC:peptide complex; and
(3) subsequently analysing T cells to identify a TCR that binds to the MHC:peptide complex.

The present disclosure also provides a method for identifying a T cell receptor (TCR) that binds to a MHC peptide complex, comprising:
(1) contacting (a) a cell comprising a MHC:peptide complex with (b) a population of T cells:
(2) incubating the cells obtained after step (1) under conditions suitable for trogocytosis of a MHC:peptide complex by a T cell comprising a TCR that binds to the MHC:peptide complex; and
(3) subsequent analysing the cells obtained after step (2) to identify a TCR that binds to the MHC:peptide complex:
wherein the MHC:peptide complex comprises a MHC molecule, and wherein the MHC molecule comprises a polypeptide comprising (i) the amino acid sequence of a MHC polypeptide, and (ii) an identifier moiety. wherein the identifier moiety is covalently associated with the polypeptide via linkage formed by a self-labelling protein tag.

The present disclosure also provides a method for identifying a T cell receptor (TCR) that binds to a MHC:peptide complex, comprising:
(1) contacting (a) a cell comprising a MHC:peptide complex with (b) a population of cells comprising T cells comprising a polypeptide comprising a sortase acceptor motif at the cell surface, in the presence of a sortase:
(2) incubating the cells obtained after step (1) under conditions suitable for interaction between the cell of (a) and the population of cells of (b); and
(3) subsequently analysing the cells obtained after step (2) to identify a T cell comprising a TCR that binds to the MHC:peptide complex, wherein the MHC:peptide complex comprises a MHC molecule, and wherein the MHC molecule comprises a polypeptide comprising (i) the amino acid sequence of a MHC polypeptide, (ii) a sortase substrate motif, and (iii) an identifier moiety. wherein the identifier moiety is covalently associated with the polypeptide via linkage formed by a self-labelling protein tag.

In some embodiments,. the sortase is provided at the cell surface of T cells comprising a polypeptide comprising a sortase acceptor motif.

In some embodiments in accordance with the various different aspects described herein relating to methods for identifying a TCR that binds to a MHC:peptide complex, the MHC polypeptide is β2 microglobulin.

In some embodiments in accordance with the various different aspects described herein relating to methods for identifying a TCR that binds to a MHC:peptide complex, the identifier moiety is a nucleic acid moiety. In some embodiments, the identifier moiety comprises or consists of single-stranded DNA (ssDNA).

In some embodiments in accordance with the various different aspects described herein relating to methods for identifying a TCR that binds to a MHC:peptide complex. the self-labelling protein tag is or comprises a HaloTag®.

In some embodiments in accordance with the various different aspects described herein relating to methods for identifying a TCR that binds to a MHC:peptide complex. the identifier moiety is covalently associated with the polypeptide via an ester bond formed by dehalogenase activity of the HaloTag® on a HaloTag® ligand comprising the identifier moiety and a chloroalkane moiety.

### DESCRIPTION

### Polypeptides

Aspects and embodiments of the present disclosure pertain to polypeptides. A ‘polypeptide’ refers to a polymer chain of a plurality of amino acid monomers linked by peptide bonds Polypeptides include peptides. which generally comprise -50 amino acids.

In some aspects and embodiments, the polypeptides of the present disclosure comprise the amino acid sequence of a Major histocompatibility complex (MHC) polypeptide, and a moiety facilitating labelling of the polypeptide with an identifier moiety.

In some aspects and embodiments, the polypeptides of the present disclosure comprise the amino acid sequence of a Major histocompatibility complex (MHC) polypeptide, and an identifier moiety, wherein the identifier moiety is covalently associated with the polypeptide via linkage formed by a self-labelling protein tag.

In some aspects and embodiments, the polypeptides of the present disclosure comprise a sortase substrate motif, and a moiety facilitating labelling of the polypeptide with an identifier moiety.

In some aspects and embodiments, the polypeptides of the present disclosure comprise a sortase substrate motif, and an identifier moiety, wherein the identifier moiety is covalently associated with the polypeptide via linkage formed by a self-labelling protein tag.

Proteins that localise to the cell membrane In some aspects and embodiments, polypeptides of the present disclosure comprise the amino acid sequence of a protein that localises to the cell membrane. Such proteins may also be referred to as cell surface proteins.

A protein that localises to the cell membrane is a protein that, when expressed by a cell (e g, a eukaryotic/mammalian cell), is detectable in or at the cell membrane. A protein that localises to the cell membrane may be detectable in or at the cell membrane e g. by analysis by immunohisto/cytochemistry or flow cytometry, e g. using an antibody to the protein

It will be appreciated that a polypeptide according to the present disclosure that comprises the amino acid sequence of a protein that localises to the cell membrane similarly localises to the cell membrane of a cell comprising/expressing the polypeptide.

In some embodiments, the protein that localises to the cell membrane is a protein expressed by an immune cell In some embodiments, an immune cell may be a cell of hematopoietic origin. e g, a neutrophil. eosinophil, basophil, dendritic cell. lymphocyte, or monocyte. A lymphocyte may be e.g., a T cell, B cell, natural killer (NK) cell, NKT cell or innate lymphoid cell (ILC), or a precursor thereof (e.g., a thymocyte or pre-B cell)

In some embodiments, the immune cell is an antigen-presenting cell (APC). APCs are cells that express MHC molecules (e.g. MHC class I and/or MHC class II molecules), and are capable of presenting MHC:peptide complexes.

APCs according to the present disclosure may be professional APCs Professional APCs are specialised for presenting antigens to T cells; they are efficient at processing and presenting MHC-peptide complexes at the cell surface, and express high levels of costimulatory molecules. Professional APCs include dendritic cells (DCs). macrophages, and B cells Non-professional APCs are other cells capable of presenting MHC-peptide complexes to T cells, in particular MHC Class 1-peptide complexes to CD8+ T cells. Accordingly, in some embodiments, the protein that localises to the cell membrane is a protein expressed by an APC (e.g., a DC, macrophage or B cell).

In some embodiments, the protein that localises to the cell membrane is an interaction partner (e.g., a ligand) for a protein that localises to the cell membrane of a T cell. A T cell according to the present disclosure may express a CD3-TCR complex In some embodiments, a T cell is a CD3+, CD4+ T cell. In some embodiments, a T cell is a CD3+, CD8+ T cell. In some embodiments, a T cell is a T helper cell (T_{H} cell). In some embodiments, a T cell is a cy to toxic T cell (e.g., a cytotoxic T lymphocyte (CTL). In some embodiments, the protein that localises to the cell membrane is an interaction partner (e.g., a ligand) for a protein selected from, a CD3-TCR complex polypeptide (e.g TCRα, TCRβ, TCRγ, TCRδ, CD3ε, CD3δ, CD3γ, CD3ζ or CD3η), CD3, CD8, CD4, CD28, PD-I, CTLA-4, LAG-3, TIM-3, VISTA, TIGIT, BTLA, OX40, 4-1BB, ICOS and CD27.

In some embodiments, the protein that localises to the cell membrane is selected from: a MHC polypeptide (e.g., a MHC polypeptide as described hereinbelow), PD-L 1, PD-L2, CD80, CD86, HVEM, B7-H3, B7-14, OX40L, 4-1BBL, ICOSL, CD40, B7RP1. CD70 and GAL9. In preferred embodiments, the protein that localises to the cell membrane is a MHC polypeptide.

### Major Histocompatibility Complex (MHC) Polypeptides

In some aspects and embodiments, polypeptides of the present disclosure comprise the amino acid sequence of a Major histocompatibility complex (MHC) polypeptide.

As used herein, an ‘MHC polypeptide’ refers to a constituent polypeptide of a MHC: molecule. A MHC molecule in turn refers to a polypeptide complex formed by non-covalent interaction between MHC polypeptides, and which is capable of binding to and presenting a peptide. Thus, a MHC polypeptide is a polypeptide capable of interacting with another MHC polypeptide to form a MHC molecule.

Polypeptide complexes according to the present disclosure may be characterised by non-covalent, protein, protein interaction between constituent polypeptide(s)/peptide(s). In some embodiments, the association comprises electrostatic interaction (e.g. ionic bonding. hydrogen bonding) and/or Van der Waals forces.

MHC molecule broadly into two classes: class I and class 11. MHC class I molecules are non-covalent heterodimers of a MHC class I alpha (α) chain polypeptide and beta (β)2 microglobulin polypeptide MHC class I α-chain polypeptide have three domains designated α1, α2 and α3. The α1 and α2 domains together form the groove to which the peptide presented by the MHC class I molecule binds, to form the MHC class I:peptide complex. MHC class I molecules are non-covalent heterodimers of a MHC class II alpha (α) chain polypeptide and a MHC class II beta (β) chain polypeptide. The α-chain comprises two domains designated α1 and α2, and the β-chain similarly comprises two domains designated β1 and β2 The α1 and β1 domains together form the groove to which the peptide presented by the MHC class II molecule binds. to form the MHC class II:peptide complex.

In humans. MHC class I and II polypeptides are encoded by polymorphic human leukocyte antigen (HLA) genes, encoding MHC complexes capable of binding to and presenting different peptides. MHC class I a chain polypeptides are encoded by HLA-A, HL4-B, HLA-C, HLA-L HLA- and HLA-G. MHC class II α chain polypeptides are encoded by HLA-DPA1, H14-DOA 1, HLA-DQA2 and HLA-DRA. MHC class II β chain polypeptides are encoded by HLA-DPB1, HLA-DQB1, HLA-DQ, HLA-DRB1, HLA-DRB3, HLA-DRB4 and HLA-DRB5. The β2 microglobulin component of the MHC class I molecule is invariant, and encoded by B2M.

In some embodiments, a MHC polypeptide may be selected from: β2 microglobulin, a MHC class I α chain polypeptide, a MHC class II α chain polypeptide. or a MHC class I β chain polypeptide. A MHC class i α chain polypeptide may be an HLA-A, HLA-B, HLA-C, HLA-E, HLA-F or HLA-G polypeptide (e.g., an HLA-A, HLA-B or HLA-C polypeptide). An HLA-A polypeptide may be an HLA-A02, HLA-A01, IHILA-A03, 1-LA-A1 1 or HLA-A24 polypeptide. A MHC class II α chain polypeptide may be an HLA-DPA1, HLA-DQAL. I-LA-DQA2 or HLA-DRA polypeptide A MHC class 11 P chain polypeptide may be an HLA-DPB1, HLA-DQB1, HLA-DQB2, HLA-DRB1, HLA-DRB3, HLA-DRB4 or HLA-DRB5 polypeptide.

In preferred embodiments, a MHC polypeptide is a MHC class I molecule polypeptide. In further preferred embodiments, a MHC polypeptide is β2 microglobulin It is advantageous to employ β2 microglobulin in the polypeptide of the present disclosure, as it provides for universal labelling of MHC class I molecules. That is, is provides for labelling of MHC class I molecules comprising a diversity of MHC class I α chain polypeptides

Reference herein to a given MHC polypeptide also encompasses isoforms, fragments, variants or homologues of the relevant polypeptide, from any species. By way of illustration, reference to β2 microglobulin includes human β2 microglobulin having the amino acid sequence shown in SEQ ID NO: I or 3, and also includes isoforms. fragments, variants or homologues of human β2 microglobulin.

As used herein, isoforms. fragments, variants or homologues of a given reference polypeptide may be characterised as having at least 70% sequence identity, preferably one of ≥80%, ≥85%, ≥90%, ≥91%, ≥92%, ≥93%, ≥94%, ≥95%, ≥96%, ≥97%, ≥98%, ≥299% or ≥100% amino acid sequence identity to the amino acid sequence of the reference polypeptide. A ‘fragment’ generally refers to a fraction of the reference protein. A ‘variant’ generally refers to a protein having an amino acid sequence comprising one or more amino acid substitutions. insertions, deletions or other modifications relative to the amino acid sequence of the reference protein, but retaining a considerable degree of sequence identity (e.g., at least 60%) to the amino acid sequence of the reference protein. An ‘isoform’ generally refers to a variant of the reference protein expressed by the same species as the species of the reference protein. A ‘homologue’ generally refers to a variant of the reference protein produced by a different species as compared to the species of the reference protein. Homologues include orthologues.

Isoforms, fragments, variants or homologues of a given reference protein may optionally be characterised as having at least 70%. preferably one of ≥80%, ≥85%, ≥90%, ≥91%, ≥92%, ≥93%, ≥94%, ≥95%, ≥96%, ≥97%, ≥98%, ≥99% or ≥100% amino acid sequence identity to the amino acid sequence of an immature or mature (i.e, after processing to remove signal peptide) form of a specified isoform of the relevant polypeptide from a given species, e.g. human.

In some aspects and embodiments according to the present disclosure, the polypeptide comprises the amino acid sequence of β2 microglobulin. In some embodiments, the amino acid sequence of β2 microglobulin comprises. or consists of, an amino acid sequence having at least 70% amino acid sequence identity, preferably one of ≥80%, ≥85%, ≥90%, ≥91%, ≥92%, ≥93%, ≥94%, ≥95%, ≥96%, 397%, ≥98%, ≥99% or ≥100% amino acid sequence identity. to SEQ ID NO 3 or 1.

Moieties Facilitating Labelling of the Polypeptide with an Identifier Moiety

In aspects and embodiments of the present disclosure. the polypeptides further comprise a moiety facilitating labelling of the polypeptide with an identifier moiety (e g, an identifier moiety as described hereinbelow).

In some embodiments, a moiety facilitating labelling of the polypeptide with an identifier moiety is or comprises an amino acid sequence forming a peptide/polypeptide moiety.

In some embodiments, the moiety facilitating labelling of the polypeptide with an identifier moiety is or comprises a self-labelling protein tag. Self-labelling protein tags are described e g. in Liss et al., Scientific Reports (2016) 5: 17740 and Wilhelm et al, Biochemistry (2021) 15 60(33): 2560-2575, both of which are hereby incorporated by reference in their entirety.

Self-labelling protein tags comprise or consist of a moiety having enzymatic activity catalysing the covalent attachment of a moiety of interest. For example, the HaloTag® is a haloalkane dehalogenase that reacts irreversibly with primary alkylhalides (e.g haloalkanes, e.g. chloroalkanes). Nucleophilic attack causes displacement of the halogen with an amino acid residue, resulting in the formation of a covalent alkyl-enzyme conjugate Specifically. on displacement of the terminal halogen an ester bond is formed between the COO⁻ group of the Asp106 of the HaloTag® and the terminal carbon of the alkane.

In some embodiments, a self-labelling protein tag is selected from a HaloTag®, SNAP-tag®, CLIP-tag. ACP tag or MCP tag. In preferred embodiments, the self-labelling protein tag is a HaloTag®.

In some embodiments, the moiety facilitating labelling of the polypeptide with an identifier moiety according to the present disclosure comprises or consists of an amino acid sequence having at least 70% amino acid sequence identity. preferably one of ≥80%, ≥85%, ≥90%, ≥91%, ≥92%, ≥93%, ≥94%, ≥95%, ≥96%, ≥97%, ≥198%, ≥199% or ≥100% amino acid sequence identity. to SEQ ID NO:4 or 5.

It will be appreciated that in some embodiments, labelling of the polypeptide according to the present disclosure with an identifier moiety modifies the moiety facilitating labelling of the polypeptide with an identifier moiety. In some embodiments, following labelling of the polypeptide with an identifier moiety, via the moiety facilitating labelling of the polypeptide with an identifier moiety. the moiety that facilitated labelling of the polypeptide is no longer suitable for facilitating (further) labelling of the polypeptide. By way of illustration, in the example of a polypeptide comprising a HaloTag K, following labelling of the polypeptide using a labelling moiety comprising a HaloTag: ligand comprising a chloroalkane moiety and an identifier moiety. the HaloTag® is no longer able to facilitate (further) labelling of the polypeptide, as the COO group of Asp106 is no longer available.

In some embodiments, the moiety facilitating labelling of the polypeptide with an identifier moiety is N-terminal to the amino acid sequence of a major histocompatibility complex (MHC) polypeptide, in the amino acid sequence of the polypeptide of the present disclosure. That is. in some embodiments, the recombinant polypeptide comprises the structure: N-term-[ . . . ]- [moiety facilitating labelling of the polypeptide with an identifier moiety]-[amino acid sequence of a MHC polypeptide]-[ . . . ]-C-term.

As used in representations of polypeptide structures herein, ‘[ . . . ]’ indicates the optional presence of further amino acid sequence(s)/protein domain(s). For example, in the structure of the final sentence of the preceding paragraph, further sequences of amino acids/protein domain(s) may optionally be present downstream of the amino acid sequence of a MHC polypeptide. before the C terminus of the polypeptide. Furthermore, as used in representations of polypeptide structures herein ‘-’ indicates an optional linker sequence. For example, in the structure of the final sentence of the preceding paragraph, a linker sequence may optionally be provided between the moiety facilitating labelling of the polypeptide with an identifier moiety and the amino acid sequence of a MHC polypeptide.

### Labelling Moieties

Aspects and embodiments of the present disclosure employ labelling moieties, e.g. for labelling of polypeptides with identifier moieties

It will be appreciated that labelling moieties are selected for compatibility with moieties facilitating labelling of the polypeptide with an identifier moiety according to the present disclosure. A labelling moiety according to the present disclosure generally comprises an identifier moiety according to the present disclosure, and a moiety for covalently attaching the identifier moiety to a polypeptide via a moiety facilitating labelling of the polypeptide with an identifier moiety.

In some embodiments, a labelling moiety according to the present disclosure is or comprises a HaloTag® ligand A Halotag® ligand comprises a moiety capable of acting as a substrate for HaloTag® haloalkane dehalogenase, for formation of a covalent alkyl-enzyme conjugate. For example, a HaloTag® ligand may comprise a primary alkylhalide moiety, e.g a haloalkane moiety (e.g., a chloroalkane moiety).

In some embodiments, a labelling moiety according to the present disclosure comprises an identifier moiety according to the present disclosure, and a chloroalkane moiety.

Identifier moieties, and moieties facilitating labelling of the polypeptide with an identifier moiety

### in Aspects and Embodiments of the Present Disclosure, the Polypeptides Comprise an Identifier Moiety

As used herein, an ‘identifier moiety’ refers to a moiety capable of serving as a moiety for identification of a polypeptide comprising the identifier moiety Identifier moieties are preferably polymorphic, providing for the labelling of a plurality of polypeptides according to the present disclosure with non-identical identifier moieties. An ‘identifier moiety’ in accordance with the present disclosure can be any detectable moiety capable of serving to distinguish a polypeptide labelled with the identifier moiety from polypeptides that are not labelled with the identifier moiety (e.g polypeptides not labelled % with an identifier moiety, or polypeptides labelled with a non-identical identifier moiety). Identifier moieties may also be referred to as ‘barcodes’, and labelling of polypeptides with non-identical identifier moieties may be referred to as ‘barcoding’.

Identifier moieties contemplated in connection with the present disclosure include e.g. nucleic acid, fluorescent, phosphorescent, luminescent, immuno-detectable (e.g. an epitope tag), radio, chemical and enzymatic labels. In preferred embodiments, the identifier moiety comprises or consists of a nucleic acid moiety That is, preferred embodiments, the identifier moiety is or comprises a nucleic acid identifier moiety.

Nucleic acids are particularly well suited to use as identifier moieties, as they are very highly polymorphic (and it is therefore possible to produce a very large diversity of unique identifier moieties), and techniques for their detection with high sensitivity and specificity are widely available (e.g. next-generation sequencing technologies).

In some embodiments, a nucleic acid moiety according to the present disclosure may comprise or consist of DNA or RNA. In some embodiments, the nucleic acid moiety comprises or consists of a polynucleotide. A ‘polynucleotide’ refers to a polymer chain of a plurality of nucleotide monomers linked by bonds between the monomers, typically phosphodiester bonds (e.g. in the case of polynucleotides formed by naturally-occurring nucleotide monomers). Polynucleotides include oligonucleotides, which generally comprise 550 nucleotides Polynucleotide may be single-stranded. or may be double-stranded (i.e. may comprise a duplex formed by hydrogen-bonding between complementary nucleotides)

In some embodiments, the nucleic acid moiety comprises or consists of single-stranded DNA (ssDNA). That is. in some embodiments, the identifier moiety is a ssDNA identifier moiety. In some embodiments, the nucleic acid moiety comprises or consists of an ssDNA polynucleotide.

In some embodiments, the ssDNA polynucleotide comprises 5 to 200 nucleotides, e g. one of 10 to 1(0, 20 to 80 or 30 to 75 nucleotides.

It will be appreciated that the nucleic acid moiety of the present disclosure is employed as an identifier moiety. The nucleic acid moiety may comprise a structure providing for detection of a polypeptide labelled with the nucleic acid moiety by analysis of the nucleic acid moiety. For example. where the nucleic acid moiety comprises or consists of a polynucleotide. the nucleic acid moiety may provide for detection by analysis of the nucleotide sequence of the polynucleotide.

The identifier moieties according to the present disclosure are preferably employed to code for (i e, as identifiers for) peptides of interest. That is. identifier moieties and peptides of interest are employed in combination, such that a given identifier moiety corresponds to a particular peptide.

The identifier moiety is useful to easily identify peptide MHC complexes presenting the peptide of interest. e.g. in a T cell following internalisation (e.g. by trogocytosis). Detection of a given identifier moiety in a T cell indicates that the T cell has internalised peptide:MHC complexes presenting the peptide it codes for.

The identifier moiety is also useful to easily identify peptide MHC complexes that have interacted with a T cell, through detection of the identifier moiety on a T cell, as a consequence of sortase-mediated transfer. In accordance with such embodiments, detection of a given identifier moiety on a T cell indicates that the T cell has interacted with peptide:MHC complexes presenting the peptide it codes for.

### Sortase-Mediated Labelling

Aspects and embodiments of the present disclosure relate to enzyme-catalysed, site-specific labelling of polypeptides with identifier moieties, articles to be employed in such labelling reactions, and the products thereof.

In particular, the present disclosure contemplates sortase-mediated transfer of identifier moieties between polypeptides. Site-specific labelling of polypeptides via sortase-catalysed transpeptidation is described e g. in Antos et al, Curr Protoc Protein Sci. (2009) CHAPTER 15 Unit-15.3, and Popp et al., Curr Protoc Protein Sci. (2009) 15:3.1-3.9. both of which are hereby incorporated by reference in their entirety.

As used herein, a ‘sortase’ refers to an enzyme that recognises and cleaves a polypeptide comprising a sortase substrate motif conforming to the consensus Leu-Pro/Ala-Xaa-Thr-Gly/Ala/Ser (SEQ ID NO. 25). Cleavage occurs between positions 4 and 5 of SEQ ID NO:25 (i.e. at the peptide bond between Thr and Gly/Ala/Ser). Sortases are useful to transfer the positions I to 4 of the sortase substrate motif, and amino acids N-terminal thereto. to a polypeptide comprising an appropriate sortase acceptor motif, via transpeptidation. Sortases and their function are reviewed e.g. in Mazmanian et al. Science (1999) 285(5428): 760-763 and Paterson and Mitchell. Trends Microbiol. (2004) 12(2): 89-95. both of which are incorporated herein by reference in their entirety.

In aspects and embodiments according to the present disclosure, a sortase is employed to catalyse transfer of a region of (a) a polypeptide comprising a sortase substrate motif (e.g., a region comprising an identifier moiety, e.g. covalently linked thereto) to (b) a polypeptide comprising a sortase acceptor motif The sortase-mediated transfer is indicative of physical interaction between polypeptides (a) and (b), as it requires their close physical proximity for efficient transfer to occur. Sortase-catalysed labelling of cells during productive immune synapses is described e.g. in Pasqual et al., Nature (2018) 553. 496-500, which is hereby incorporated by reference in its entirety.

Based on sequence alignment and phylogenetic analysis of 61 sortases from gram-positive bacterial genomes, sortases have been divided into four classes: sortase A, sortase B. sortase C. and sortase D (see Dramsi et al., Res Microbiol.(2005) 156(3):289-97).

Sortases include sortase A, which is identified by Enzyme Commission number 3 4.22.70. Sortase A encompasses sortase A from Staphylococus aureus (also referred to as ‘Sa-SrtA’), having the amino acid sequence shown in SEQ ID NO. 19. The region of S, aureus sortase A conferring sortase activity is shown in SEQ ID NO 20. Sortase A also encompasses sortase A from Streptococcus pyogenes (also referred to as Sp-SrtA), having the amino acid sequence shown in SEQ ID NO. 23. The region of 'S. pyogenes sortase A conferring sortase activity is shown in SEQ ID NO. 24.

The present disclosure also contemplates sortase variants. Sortase variants are described e.g in Dorr et a. PNAS US (2014) 111(37): 13343-13348; Chen et al., PNAS USA (201 1) 108(28) 11399-114(4-; and Chen et al. Sci Rep (2016) 6 31899, all of which are hereby incorporated by reference in their entirety.

In some embodiments in accordance with various aspects of the present disclosure, a sortase is selected from: sortase A (EC 3 4.22.70). S aureus sortase A or a variant thereof, and S. pyogenes sortase A or a variant thereof.

A variant of sortase A from Staphylococcus aureus (i.e. a variant of S aureus sortase A) refers to a polypeptide having at least 70% amino acid sequence identity e.g. one of ≥75%, ≥280%, ≥85%, 90%, ≥91%, ≥92%, ≥93%, ≥94%, ≥9%, ≥96%, ≥97%, ≥98% or ≥99% amino acid sequence identity to the amino acid sequence of SEQ ID NO: 19. Similarly, variant of sortase A from Streptococcus pyogenes (i.e. a variant of S pyogenes sortase A) refers to a polypeptide having at least 70% amino acid sequence identity e.g. one of ≥75%, ≥80%, ≥85%,≥90%, ≥91%, ≥292% ≥93%, ≥94%, ≥95%, ≥96%, ≥97%, ≥98% or ≥99% amino acid sequence identity to the amino acid sequence of SEQ ID NO:23. It will be appreciated that variants of S aureus sortase A and variants of S. pyogenes sortase A preferably retain sortase activity.

It will be appreciated that a sortase variant comprises one or more (e g. one of 1, 2, 3, 4, 5, 6, 7. 8, 9 or 10. or more) modifications relative to the amino acid sequence of a reference sortase (e.g. S aureus sortase A. S pyogenes sortase A).

A ‘modification’ refers to a difference relative to a reference amino acid sequence. A reference amino acid sequence may be the amino acid sequence encoded by the most common nucleotide sequence of the gene encoding the relevant protein. In embodiments herein (and also in the art 5 more generally), a ‘modification’ may also be referred to as a ‘substitution’ or a‘mutation’. A modification typically comprises substitution of an amino acid residue with a non-identical ‘replacement’ amino acid residue. A replacement amino acid residue of a modification according to the present disclosure may be a naturally-occurring amino acid residue (i.e. encoded by the genetic code) which is non-identical to the amino acid residue at the relevant position of the amino acid sequence prior to modification, selected from: alanine (Ala), arginine (Arg), asparagine (Asn), aspartic acid (Asp), cysteine (Cys), glutamine (Gin), glutamic acid (Glu), glycine (Gly), histidine (His), isoleucine (Ile), leucine (Leu), lysine (Lys), methionine (Met), phenylalanine (Phe), proline (Pro), serine (Ser), threonine (Mhr), tryptophan (Trp), tyrosine (Tyr), and valine (Val) In some embodiments, a replacement amino acid residue of a modification may be a non-naturally occurring amino acid residue i.e. an amino acid residue other than those recited in the preceding sentence. Examples of non-naturally occurring amino acid residues include norleucine, omithine. norvaline. homoserine, aib, and other amino acid residue analogues such as those described in Ellman, et al., Meth. Enzym. 202 (1991) 301-336.

In some embodiments, a sortase variant according to the present disclosure comprises an amino acid sequence having at least 70% amino acid sequence identity to SEQ ID NO:19, 20, 23 or 24. and comprises one or more (e.g. one of 1. 2. 3, 4, 5, 6, 7, 8. 9 or 10, or more) modifications relative to SEQ ID NO: 19, 20, 23 or 24.

In some embodiments, a sortase variant comprises one or more (e.g. one of 1. 2. 3. 4, 5, 6, 7, 8, 9 or 10, or more) of the following modifications relative to SEQ ID NO:19 or SEQ ID NO:20: K196T, P86L, P94S, P94R, N98S, A I(4T, El1060, A118T, F122S, F122Y. Dl24G, N127S, K134R, F154R, D160N, D165A, K173E, GI74S, K177E, I 182V and/or K190E In some embodiments, a sortase variant comprises K196T relative to SEQ ID NO:19 or SEQ ID NO:20. It will be appreciated that the modifications of the preceding sentence are numbered according to SEQ ID NO:19, and that when the relevant modification is instead provided in SEQ ID NO 20, the corresponding position of SEQ ID NO. 20 is instead modified By way of illustration, when the modification ‘K196T’ is provided in SEQ ID NO:20, ‘K’ at position 1 37 of SEQ ID NO:20 is replaced with ‘T’.

In some embodiments in accordance with various aspects of the present disclosure, a sortase comprises. or consists of, an amino acid sequence having the amino acid sequence of SEQ ID NO:19, 20. 21. 22, 23 or 24, or an amino acid sequence having at least 70% amino acid sequence identity, e.g. one of ≥75%, ≥80%, ≥85%, ≥90%, ≥91%, ≥92%, ≥93%, ≥94%, ≥295%, ≥196%, ≥97%, ≥98% or ≥99% sequence identity to SEQ ID NO:19, 20, 21. 22, 23 or 24. In some embodiments, a sortase comprises. or consists of, an amino acid sequence having the amino acid sequence of SEQ ID NO:19, or an amino acid sequence having at least 70% amino acid sequence identity, e.g. one of ≥75%, ≥80% =85%, ≥90%, ≥91%, ≥92%, ≥93%, ≥94%, ≥95%, ≥96%. 297%, ≥98% or ≥99% sequence identity to SEQ ID NO. 19 In some embodiments, a sortase comprises. or consists of, an amino acid sequence having the amino acid sequence of SEQ ID NO. 20, or an amino acid sequence having at least 70% amino acid sequence identity. e.g. one of 275%, ≥80%, ≥85%, ≥90%, ≥91%, ≥92%, ≥93%, ≥94%, ≥95%, ≥96%, ≥97%, ≥98% or ≥299% sequence identity to SEQ ID NO: 20. In some embodiments, a sortase comprises, or consists of. an amino acid sequence having the amino acid sequence of SEQ ID NO:21. or an amino acid 15 sequence having at least 70% amino acid sequence identity. e.g. one of 75% ≥80%, ≥85%, 290%, ≥91%, ≥92%, ≥93%, ≥94%, ≥95%, ≥96%, ≥97%, ≥98% or, 99% sequence identity to SEQ ID NO: 21. In some embodiments, a sortase comprises, or consists of, an amino acid sequence having the amino acid sequence of SEQ ID NO:22, or an amino acid sequence having at least 70% amino acid sequence identity. e.g. one of ≥75%, ≥80%, ≥85%, ≥90%, ≥91%, ≥92%, ≥93%, ≥94%, ≥95%. =96%. =97%, ≥98% or ≥99% sequence identity to SEQ ID NO:22. In some embodiments, a sortase comprises. or consists of, an amino acid sequence having the amino acid sequence of SEQ ID NO:23, or an amino acid sequence having at least 70% amino acid sequence identity. e.g. one of ≥75%, ≥80%, ≥85%, ≥90%, ≥91%, ≥292%, ≥93%, ≥94%, ≥95%, ≥96%. 297%, ≥98% or ≥99% sequence identity to SEQ ID NO:23 In some embodiments, a sortase comprises. or consists of, an amino acid sequence having the amino acid sequence of SEQ ID NO:24, or an amino acid sequence having at least 70% amino acid sequence identity, e.g. one of 275%, ≥80%, ≥85%, ≥90%, ≥91%, ≥92, 93%, ≥94%, ≥95%, ≥96%, ≥97%, ≥98% or ≥299% sequence identity to SEQ ID NO: 24.

In some embodiments in accordance with various aspects of the present disclosure, a sortase substrate motif comprises. or consists of, a substrate motif for a sortase selected from sortase A (EC 3.4.22 70). S aureus sortase A or a variant thereof, and S. pyogenes sortase A or a variant thereof.

Sortase substrate motifs include those conforming to the consensus Leu-Pro-Xaa-Thr-Gly (SEQ ID NO:26). which serves as a substrate for S aureus sortase A and S. pyogenes sortase A, and those conforming to the consensus Leu-Pro-Xaa-Thr-Ala (SEQ ID NO:27) or Leu-Pro-Xaa-Thr-Ser (SEQ ID N0:28), which serve as substrates for S. pyogenes sortase A (see e.g. Johnson et al., J Biol Chem. (2022) 298(10) 102446) Herein, where a given sortase substrate motif is said to ‘serve as a substrate’ for a given reference sortase, the given reference sortase is capable of catalysing clear age of the sortase substrate motif (i.e. at the peptide bond between positions 4 and 5 of SEQ ID NO:25).

Accordingly, in some embodiments, a sortase substrate motif comprises. or consists of, an amino acid sequence conforming to the consensus sequence of one of SEQ ID NOs. 25, 26. 27 or 28. In some embodiments, a sortase substrate motif comprises, or consists of, an amino acid sequence conforming to the consensus sequence of SEQ ID NO. 25 In some embodiments, a sortase substrate motif comprises. or consists of, an amino acid sequence conforming to the consensus sequence of SEQ ID NO. 26. In some embodiments, a sortase substrate motif comprises. or consists of, an amino acid sequence conforming to the consensus sequence of SEQ ID NO:27. In some embodiments, a sortase substrate motif comprises. or consists of, an amino acid sequence conforming to the consensus sequence of SEQ ID NO:28.

In some embodiments, a sortase substrate motif may be provided at. or proximal to, the C-terminus of a polypeptide comprising the sortase substrate motif. That is, in some embodiments, position I of the sequence conforming to the consensus sequence of SEQ ID NO:25 is provided within 50 amino acids, e.g. within one of 40, 30. 25, 20. 15. 10 or 5 amino acids of the C-terminus of the polypeptide.

Sortases catalyse the transfer of polypeptide/peptide moieties from proteins comprising a sortase substrate motif for the sortase, to proteins comprising a sortase acceptor motif for the sortase. Sortase acceptor motifs are typically oligopeptide sequences of small, non-polar. hydrophobic amino acids (e.g. glycine or alanme), provided at the N-terminus of a polypeptide/peptide comprising the oligopeptide. The best-characterised sortase acceptor motif, which serves as an acceptor motif for e.g S aureus sortase A, is an N-terminal oligoglycine.

Herein, where a given sortase acceptor motif is said to ‘serve as an acceptor motif for a given reference sortase, the given sortase acceptor motif is capable of serving as a nucleophile for the intermediate formed by cleavage of a polypeptide/peptide comprising an appropriate sortase substrate motif for the given reference sortase by the sortase, i.e. in the context of a transpeptidation reaction.

In some embodiments, a sortase acceptor motif maty comprise, or consist of, at least 1. 2. 3, 4, 5. 6, 7, 8, 9 or 10 (e g. 2 to 10, e.g. 4) amino acids provided at the N-terminus of a polypeptide/peptide comprising such a motif, wherein the amino acids are selected from glycine and alanine.

In some embodiments, a sortase acceptor motif may comprise, or consist of, Gₙ provided at the N-terminus of a pols peptide/peptide comprising the Gₙ i.e. ‘N-term-G’) wherein ‘n’ is at least 1. 2. 3, 4, 5, 6. 7, 8. 9 or 10. In some embodiments. ‘n’ is at least 2. In some embodiments, ‘n’ is 2 to 10. In some embodiments, ‘n’ is 4. The sortase acceptor motif ‘N-term-G₄’ serves as an acceptor motif for S aureus sortase A and S. pyogenes sortase A.

In some embodiments, a sortase acceptor motif may comprise, or consist of, An provided at an N-terminus (i e ‘N-term-An ) wherein ‘n’ is at least 1. 2, 3, 4, 5. 6, 7. 8. 9 or 10. In some embodiments. ‘n’ is at least 2. In some embodiments, ‘n’ is 2 to 10. In some embodiments. ‘n’ is 4.

It will be appreciated that where an aspect or embodiment of the present disclosure relates to (i) a polypeptide comprising a sortase substrate motif and (ii) a sortase. the sortase substrate motif and sortase are preferably selected to be compatible with one another. Similarly, where an aspect or embodiment relates to (i) a polypeptide comprising a sortase acceptor motif and (ii) a sortase, the 25 sortase acceptor motif and sortase are preferably selected to be compatible with one another.

Similarly, where an aspect or embodiment relates to (i) a polypeptide comprising a sortase substrate motif, (ii) a polypeptide comprising a sortase acceptor motif, and (iii) a sortase. the sortase substrate motif, the sortase acceptor motif and sortase are preferably selected to be compatible with one another.

A given sortase substrate motif and a given sortase are compatible if the sortase catalyses cleavage of the sortase substrate motif (i e at the peptide bond between positions 4 and 5 of SEQ ID NO:25). By way of illustration, the sortase consisting of the amino acid sequence of SEQ ID NO. 22 catalyses cleavage of a polypeptide/peptide comprising a sortase substrate motif conforming to the consensus of SEQ ID NO:26, and so this combination of sortase substrate motif and sortase is compatible.

A given sortase acceptor motif and a given sortase are compatible if the sortase acceptor motif serves as a nucleophile for the intermediate formed by cleavage of a polypeptide/peptide comprising an appropriate sortase substrate motif for the sortase by the sortase By way of illustration, the sortase acceptor motif ‘N-term-G₄′ serves as a nucleophile for the intermediate formed by cleavage of a polypeptide/peptide comprising an appropriate sortase substrate motif for the sortase consisting of the amino acid sequence of SEQ ID NO: 022, and so this combination of sortase acceptor motif and sortase is compatible.

A given sortase substrate motif, a given sortase acceptor motif and a given sortase are compatible if the sortase catalyses cleavage of the sortase substrate motif, and the sortase acceptor motif serves as a nucleophile for the intermediate formed by cleavage of the sortase substrate motif by the sortase. By way of illustration, the sortase acceptor motif ‘N-term-G₄′ serves as a nucleophile for the intermediate formed by cleavage of a polypeptide/peptide comprising a sortase substrate motif conforming to the consensus of SEQ ID NO:26 by the sortase consisting of the amino acid sequence of SEQ ID NO:22, and so this combination of sortase acceptor motif, sortase substrate motif and sortase is compatible.

It will be appreciated that in aspects and embodiments wherein a polypeptide comprises a sortase substrate motif. the sortase substrate motif is positioned within the amino acid sequence of the polypeptide such as to permit sortase-mediated transfer of an identifier moiety, when the polypeptide comprises such moiety (e.g. following covalent association of the identifier moiety with the polypeptide via linkage formed by a self-labelling protein tag). That is. in embodiments wherein a polypeptide comprises a sortase substrate motif and a moiety facilitating labelling of the polypeptide with an identifier moiety (e.g. a self-labelling protein tag. e.g. a HaloTag®), the sortase substrate motif is provided downstream of (i e C-terminal to) the moiety facilitating labelling of the polypeptide with an identifier moiety, in the amino acid sequence of the polypeptide. Similarly, in embodiments wherein a polypeptide comprises a sortase substrate motif and an identifier moiety covalently associated with the polypeptide via linkage formed by a self-labelling protein tag (e g, a Halotag®), the sortase substrate motif is provided downstream of (i.e. C-terminal to) the self-labelling protein tag. That is, in some embodiments, the polypeptide comprises the structure. N-term-[ . . . ]-[moiety facilitating labelling of the polypeptide with an identifier moiety[-] sortase substrate motif]-[ . . . ]-C-term.

Similarly, it will be appreciated that in aspects and embodiments wherein a polypeptide comprises a sortase acceptor motif, the sortase acceptor motif is positioned within the amino acid sequence of the polypeptide such as to permit sortase-mediated transfer of an identifier moiety to the polypeptide. That is. where a polypeptide comprises a sortase acceptor motif, the sortase acceptor motif is provided at the N-terminus of the polypeptide.

Aspects and embodiments according to the present disclosure provide a cell comprising a polypeptide. wherein the polypeptide comprises a sortase acceptor motif. In preferred embodiments, the cell is a T cell. A T cell according to the present disclosure may express a CD3-TCR complex. In some embodiments, the T cell is a CD3+, CD4+ T cell. In some embodiments, the T cell is a CD3+, CD8+ T cell. In some embodiments, the T cell is a T helper cell (Tn cell) In some embodiments, the T cell is a cytotoxic T cell (e.g a cytotoxic T lymphocyte (CTL))

In some embodiments, the T cell comprises a polypeptide comprising a sortase acceptor motif at its cell surface. That is. in some embodiments, a polypeptide comprising a sortase acceptor motif is present in or at the cell surface of the T cell.

The T cell may comprise a polypeptide comprising a sortase acceptor motif as a consequence of modification of the T cell to compose the polypeptide. In some embodiments, the T cell may comprise a polypeptide comprising a sortase acceptor motif as a consequence of modification of the T cell to comprise nucleic acid encoding a polypeptide comprising a sortase acceptor motif (e.g. using a suitable genetic engineering platform)

However. in preferred embodiments, the T cell is modified to comprise a polypeptide comprising a sortase acceptor motif through non-genetic modification of the T cell For example, the polypeptide may be non-genetically bioconjugated to the T cell. Non-genetic bioconjugation techniques are reviewed e.g. in Roy el al. Bioconjug Chem. (2020) 31(11): 2465-2475, which is hereby incorporated by reference in its entirety. Advantageously, such techniques do not require genetic engineering to produce a T cell comprising a polypeptide comprising a sortase acceptor motif.

In some embodiments, a T cell is a non-genetically -modified T cell. In some embodiments, a T cell according to the present disclosure does not comprise exogenous nucleic acid. Non-genetic bioconjugation strategies typically comprise attaching exogenous functional groups to the cell membrane, without changing the genetic nature of the cell.

A polypeptide comprising a sortase acceptor motif may be attached to the surface of a T cell through conjugation to a functional group introduced into the T cell metabolically Azide, alkyne or ketone moieties can be introduced into glycans presented at the cell surface through metabolic glycan labelling (MGL).

In some aspects and embodiments, the present disclosure provides a T cell comprising a polypeptide comprising a sortase acceptor motif, wherein the polypeptide is linked to the cell surface of the T cell via metabolic glycan labelling of the T cell followed by ‘Click’ chemistry-based conjugation of the polypeptide. The polypeptide may be referred to as being, or having been, ‘immobilised’ on the T cell. i.e. on or at the cell surface of the T cell.

In some embodiments, a polypeptide comprising a sortase acceptor motif is provided at the cell surface of the T cell as a consequence of covalent association of the polypeptide with a molecule in or at the cell membrane of the T cell. In some embodiments, a polypeptide comprising a sortase acceptor motif is provided at the cell surface of the T cell as a consequence of non-covalent interaction between the polypeptide and a molecule in or at the cell membrane of the T cell.

For example, sialic acid residues of cell surface glycans can be modified to comprise azide moieties through culturing cells in cell culture medium comprising N-azidoacetylmannosamine (ManNAz). A polypeptide comprising a sortase acceptor motif and functionalised with a cycloalkyne moiety (e.g. a dibenzocyclooctyl (DBCO) moiety or an azadibenzocyclooctvne (ADIBO) moiety) may thereafter be conjugated to azide-modified cell surface glycans via strain-promoted alkyne-azide cycloaddition (SPAAC).

Accordingly, in some embodiments, the polypeptide comprising a sortase acceptor motif further comprises a moiety suitable for conjugation of the polypeptide to an azide moiety-labelled interaction partner via SPAAC. In some embodiments, the moiety suitable for conjugation of the polypeptide to an azide moiety-labelled interaction partner via SPAAC is or comprises a cycloalkyne moiety (e.g. a DBCO moiety or an azadibenzocyclooctyne (ADIBO) moiety). In some embodiments, the cycloalkyne moiety is or comprises a DBCO moiety.

In some aspects and embodiments, the present disclosure provides a T cell comprising a polypeptide comprising a sortase acceptor motif, wherein the polypeptide is conjugated to the cell surface of the T cell. via a SPAAC reaction between a cycloalkyne moiety (e.g. a DBCO moiety or an ADIBO moiety) and an azide-modified cell surface glycan.

In some aspects and embodiments, the T cell further comprises a sortase, e.g. a sortase according to an embodiment described hereinabove. Thus, aspects and embodiments according to the present disclosure provide a T cell comprising (i) a polypeptide comprising a sortase acceptor motif, and (ii) a sortase.

The T cell may comprise a sortase as a consequence of modification of the T cell to comprise the sortase In some embodiments, the T cell may comprise a sortase as a consequence of modification of the T cell to compose nucleic acid encoding a sortase (e.g. using a suitable genetic engineering platform).

However, in preferred embodiments, the T cell is modified to comprise a sortase through non-genetic modification of the T cell. In some embodiments, the T cell is modified to comprise a sortase via bioconjugation, e.g. via metabolic glycan labelling of the T cell followed by ‘Click’ chemistry-based conjugation of the sortase. The sortase may be referred to as being, or having been. ‘immobilised’ on the T cell. i.e. on or at the cell surface of the T cell.

In some embodiments, a sortase is provided at the cell surface of the T cell as a consequence of covalent association of a sortase, or of a polypeptide comprising a sortase moiety, with a molecule in or at the cell membrane of the T cell. In some embodiments, a sortase, or a polypeptide comprising a sortase moiety, is provided at the cell surface of the T cell as a consequence of non-covalent interaction between the sortase/polypeptide comprising a sortase moiety, and a molecule in or at the cell membrane of the T cell. Such non-covalent interaction may be protein:protein interaction. In some embodiments, the interaction comprises electrostatic interaction (e.g. ionic bonding. hydrogen bonding) and/or Van der Waals forces. Such non-covalent interaction may be non-covalent interaction of the kind observed in antibody:antigen interaction.

In some embodiments, the T cell is modified to comprise a sortase via metabolic gly can labelling of the T cell, ‘Click’ chemistry-based conjugation of a hapten, and application of an antigen-binding molecule that binds to the hapten, wherein the antigen-binding molecule comprises or is linked to the sortase. In some embodiments, a hapten may be selected from DOTAM, DOTA. digoxigenin, biotin and fluorescein. In some embodiments, a hapten is DOTAM or DOTA.

For example, sialic acid residues of cell surface glycans can be modified to comprise azide moieties through culturing cells in cell culture medium comprising ManNAz A molecule comprising a cycloalkyne moiety (e.g a DBCO moiety or an ADIBO moiety) conjugated to a hapten (e.g. DOTAM or DOTA) may thereafter be employed to conjugate the hapten to the azide-modified cell surface glycans via SPAAC. An antigen-binding molecule that binds specifically to the hapten, and which comprises a sortase (i.e. as a fusion polypeptide). may thereafter be applied to the cells, thereby non-covalently modifying the cells to comprise the sortase at the cell surface.

In some aspects and embodiments, the present disclosure provides a T cell comprising a hapten (e.g. DOT AM or DOTA) conjugated to the cell surface of the T cell, via a SPAAC reaction between a cycloalkyne moiety (e.g. a DBCO moiety or an ADIBO moiety) and an azide-modified cell surface glycan In some aspects and embodiments, the present disclosure provides a T cell comprising a hapten (e.g. DOTAM or DOTA) conjugated to the cell surface of the T cell, via a SPAAC reaction between a cycloalkyne moiety (e.g. a DBCO moiety or an ADIBO moiety) and an azide-modified cell surface glycan, further comprising an antigen-binding molecule bound specifically to the hapten, wherein the antigen-binding molecule comprises a sortase.

The present disclosure also provides a compound comprising a hapten (e.g. DOTAM or DOTA) and a cycloalkyne moiety (e.g. a DBCO moiety or an ADIBO moiety).

In some aspects and embodiments, the present disclosure provides a compound comprising a hapten (e.g. DOTAM or DOTA) conjugated with a cycloalkyne moiety (e.g. a DBCO moiety), via a linkage. In some embodiments, the linkage is. or comprises, a thiourea moiety. Conjugation of the hapten with the cycloalksne moiety is thus achieved by forming a thiourea bond. for example by reaction of an isothiocyanate (NCS) group with a primary amine group.

Accordingly, present disclosure provides a compound of structure (I):

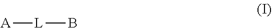

wherein A is a hapten, L is a linkage, and B is a cycloalkyne moiety In some embodiments, the linkage L is. or comprises, a thiourea moiety.

In some embodiments, the hapten is. or comprises, DOTAM. In some embodiments, DOTAM comprises a C-functionalised cyclen moiety. In some embodiments, the C-functionalised cy clen moiety comprises a NCS group.

Accordingly, in some embodiments, the hapten comprises, or consists of. structure (II),

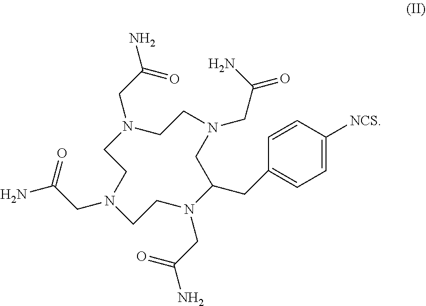

In some embodiments, the hapten comprises DOTA. In some embodiments. DOTA comprises a C-functionalised cyclen moiety In some embodiments, the C-functionalised cyclen moiety comprises a NCS group. Accordingly, in certain embodiments, the hapten comprises structure I

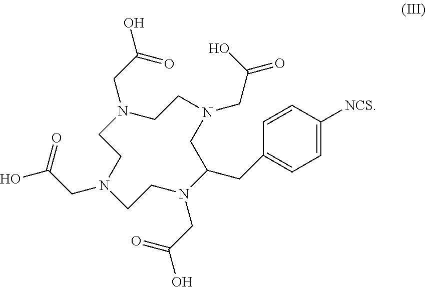

In some aspects and embodiments, the cycloalkyne moiety comprises an amine group. In some embodiments, the cycloalkyne moiety comprises DBCO-amine. DBCO-amine has structure IV:

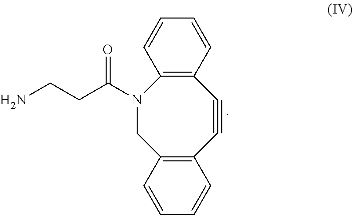 [SMILES: NCCC(=O)N1Cc2ccccc2C#Cc2ccccc21]

In preferred embodiments, the compound comprises DOTAM conjugated to DBCO via a thiourea bond. In some embodiments, the compound has structure V:

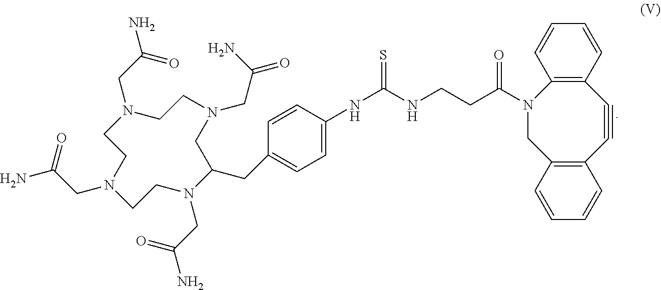 [SMILES: NC(=O)CN1CCN(CC(N)=O)CCN(CC(N)=O)C(Cc2ccc(NC(=S)NCCC(=O)N3Cc4ccccc4C#Cc4ccccc43)cc2)CN(CC(N)=O)CC1]

In certain embodiments, the compound comprises DOTA conjugated to DBCO via a thiourea bond. In some embodiments, the compound has structure VI:

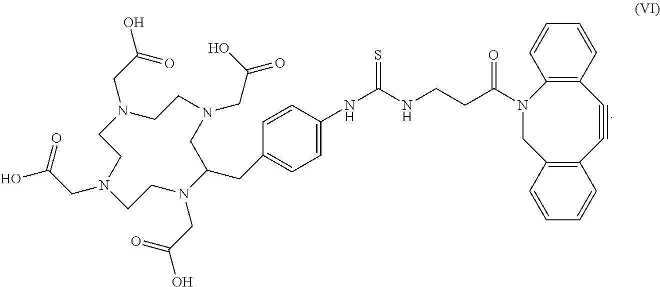 [SMILES: O=C(O)CN1CCN(CC(=O)O)CCN(CC(=O)O)C(Cc2ccc(NC(=S)NCCC(=O)N3Cc4ccccc4C#Cc4ccccc43)cc2)CN(CC(=O)O)CC1]

It will be understood that references herein to compounds include, where appropriate salts, hydrates and solvates thereof.

In aspects and embodiments of the present disclosure., a sortase is provided as a fusion protein with a polypeptide of an antigen-binding molecule. In such aspects and embodiments, the antigen-binding molecule may be described as comprising a sortase moiety.

Accordingly. the present disclosure also provides an antigen-binding molecule comprising a sortase moiety. The present disclosure also provides a nucleic acid, or a plurality of nucleic acids, encoding such an antigen-binding molecule. Also provided is an expression vector. or a plurality of expression vectors, comprising a nucleic acid or plurality of nucleic acids encoding such an antigen-binding molecule. Also provided is a cell comprising such an antigen-binding molecule, such a nucleic acid or plurality of nucleic acids, or such an expression vector or plurality of expression vectors. Also provided is a composition comprising such an antigen-binding molecule. such a nucleic acid or plurality of nucleic acids. such an expression vector or plurality of expression vectors, or such a cell.

The sortase moiety may comprise, or may consist of, a sortase or variant thereof according to any embodiment described herein.

As used herein, an ‘antigen-binding molecule’ refers to a molecule that binds to a given target antigen. Antigen-binding molecules include antibodies (i.e. immunoglobulins (Igs)), and antigen-binding fragments and derivatives thereof. In some embodiments, an antigen-binding molecule according to the present disclosure comprises. or consists of, a monoclonal antibody, a monospecific antibody, a multispecific (e.g., bispecific, trispecific. etc.) antibody, a variable fragment (Fv) molecule, a single-chain Fv (scFv) molecule, a fragment antigen-binding (Fab) molecule, a single-chain Fab molecule (scFab), a crossFab molecule, a Fab’ molecule, a Fab′-SH molecule, a F(ab′)₂ molecule, a diabodA molecule, a triabody molecule, an scFv-Fc molecule, a minibody molecule, a heavy chain only antibody (HCAb) molecule. or a single domain antibody (dAb, VHH) molecule.

Antigen-binding molecules according to the present disclosure also include further target antigen-binding peptides/polypeptides such as peptide aptamers. thioredoxins, anticalins., Kunitz domains, avimers, knottins, fynomers, atrimers, DARPins, affibodys, affilins, armadillo repeat proteins (ArmRPs). OBodys and adnectins (reviewed e.g. in Reverdatto et al., Curr Top Med Chem. 2015. 15(12). 1082-1101. which is hereby incorporated by reference in its entirety (see also e.g. Boersma et al., J Biol Chem (2011) 286:41273-85 and Emanuel era., Mabs (2011) 3 38-44)) Antigen-binding molecules according to the present disclosure also include target antigen-binding nucleic acids, e.g. nucleic acid aptamers (reviewed, for example, in Zhou and Rossi Nat Rev Drug Discov 2017 16(3):181-202). Antigen-binding molecules according to the present disclosure also include target antigen-binding small molecules (e g. low molecular weight (<1000 daltons, typically between ˜300-700 daltons) organic compounds).

In some embodiments, an antigen-binding molecule according to the present disclosure is. or comprises, an antigen-binding peptide/polypeptide, or an antigen-binding peptide/polypeptide complex An antigen-binding molecule may comprise more than one peptide/polypeptide that together form an antigen-binding molecule. The peptides/polypeptides may associate covalent or non-covalently In some embodiments, the peptides/polypeptides form part of a larger polypeptide comprising the peptides/polypeptides (e.g. in the case of an scFv molecule comprising a VH region and a VL region, or in the case of a scFab molecule comprising VH-CH1 and VL-CL).

In some embodiments, the antigen-binding molecule comprises an antibody heavy chain variable (VH) region and an antibody light chain variable (VL) region of an antibody capable of binding to a given target antigen In some embodiments, the antigen-binding molecule comprises. or consists of, an Fv molecule formed by the VH region and a VL region of an antibody capable of binding to a given target antigen. In some embodiments, the VH region and a VL region may be provided in the same polypeptide, and joined by a linker sequence. In some embodiments, the antigen-binding molecule comprises. or consists of, an scFv molecule that binds to a given target antigen.

Antigen-binding molecules of the present disclosure general comprise six complementarity-determining regions CDRs; three in the heavy chain variable (VI-1) region: HC-CDR 1, HC-CDR2 and HC-CDR3, and three in the light chain variable (VL) region: LC-CDR1, LC-CDR2. and LC-CDR3. The six CDRs together define the paratope of the antigen-binding molecule. which is the part of the molecule that binds to the target antigen The VH region and VL region comprise framework regions (FRs) either side of each CDR, which provide a scaffold for the CDRs. From N-terminus to C-terminus. VH regions comprise the following structure: N term-[HC-FR1]-[IC-CDR1]-[HC-FR2]-[H1C-CDR2]-[H1C-FR3]-[-IC-CDR3]-[IC-FR4]-C term; and VL regions comprise the following structure: N term-[LC-FR1]-[LC-CDRI]-[LC-FR2]-[LC:-CDR2]-[LC-FR3]-[LC-CDR3]-[LC-FR4]-C term There are several different conventions for defining antibody CDRs and FRs, such as (i) the Kabat system, described in Kabat et al., Sequences of Proteins of Immunological Interest, 5th Ed Public Health Service. National Institutes of Health, Bethesda. MD (1991); (ii) the Chothia system, described in Chothia et ai., J. Mol. Biol. 196:901-917 (1987); and (iii) the international IMGT (ImMunoGeneTics) information system (LeFranc et al., Nucleic Acids Res. (2015) 43 (Database issue).D413-22). which uses the IMGT V-DOMAIN numbering rules as described in Lefranc et al., Dev Comp. Immunol. (2003) 27:55-77 In some embodiments, the CDRs and FRs of the VH regions and VL regions of the antigen-binding molecules described herein are defined according to the Kabat system, the Chothia system or the IMGT information system.

In some embodiments, the antigen-binding molecule of the present disclosure binds to a hapten, e.g. DOTAM or DOTA. In some embodiments, the antigen-binding molecule comprises the CDRs of an antigen-binding molecule that binds to a hapten (e.g. a hapten as described herein, e.g. DOTAM or DOT A). In some embodiments, the antigen-binding molecule comprises the FRs of an antigen-binding molecule that binds to a hapten (e g a hapten as described herein, e g. DOTAM or DOTA). In some embodiments, the antigen-binding molecule comprises the CDRs and the FRs of an antigen-binding molecule that binds to a hapten (e.g. a hapten as described herein. e.g. DOTAM or DOTA). That is, in some embodiments, the antigen-binding molecule comprises the VH region and the VL region of an antigen-binding molecule that binds to a hapten (e.g a hapten as described herein, e.g. DOTAM or DOTA).

In some embodiments, the antigen-binding molecule comprises the CDRs, FRs and/or the VH and/or VL regions of a DOTAM-binding antibody, or CDRs. FRs and/or VH and/or VL regions which are derived from those of a DOTAM-bonding antibody. In some embodiments, the antigen-binding molecule comprises the CDRs. FRs and/or the VH and/or VL regions of a DOTAM-binding antibody described in WO 2019/202399 A1. or CDRs, FRs and/or VH and/or VL regions which are derived from those of a DOTAM-binding antibody described in WO 2019/202399 A1.

WO 2019/202399 A1 describes DOTAM-binding antibodies such as PRIT-213, the antigen-binding region of which is formed of a VH region having the amino acid sequence of SEQ ID NO. 33 (SEQ ID NO:7 of WO 2019/202399 A1) and a VL region having the amino acid sequence of SEQ ID NO 41 (SEQ ID NO. 8 of WO 2019/202399 A 1). In some embodiments, the antigen-binding molecule comprises the CDRs, FRs and/or the VH and/or VL regions of PRIT-213, or CDRs. FRs and/or VH and/or VL regions which are derived from those of PRIT-213.

In some embodiments, the antigen-binding molecule comprises:
(1) a VH region incorporating the following CDRs:
   HC-CDR1 having the amino acid sequence of SEQ ID NO:34
   HC-CDR2 having the amino acid sequence of SEQ ID NO. 35
   HC-CDR3 having the amino acid sequence of SEQ ID NO. 36,

or a variant thereof in which 1 or 2 or 3 amino acids in HC-CDR1, and/or in which 1 or 2 or 3 amino acids in HC-CDR2, and/or in which 1 or 2 or 3 amino acids in HC-CDR3 are substituted with another amino acid.

In some embodiments, the antigen-binding molecule comprises:
(2) a VH region incorporating the following FRs:
   HC-FR 1 having the amino acid sequence of SEQ ID NO:37
   HC-FR2 having the amino acid sequence of SEQ ID NO:38
   HC-FR3 having the amino acid sequence of SEQ ID NO. 39
   HC-FR4 having the amino acid sequence of SEQ ID NO:40.
   or a variant thereof in which I or 2 or 3 amino acids in HC-FR 1, and/or in which 1 or 2 or 3 amino acids in HC-FR2, and/or in which I or 2 or 3 amino acids in HC-FR3, and/or in which I or 2 or 3 amino acids in HC-FR4 are substituted with another amino acid.

In some embodiments, the antigen-binding moiety comprises a VH region comprising the CDRs according to (1) and the FRs according to (2).

In some embodiments, the antigen-binding molecule comprises:
(3) a VH region comprising an amino acid sequence having at least 70%,0 sequence identity, more preferably one of at least ≥75%, ≥80%, ≥85%, ≥86%, ≥287%, ≥88%, ≥89%, ≥290%, ≥91%, ≥92%, 293%, ≥294%, ≥2.95%, ≥96%, ≥97%, ≥98%, ≥9% or ≥100%, sequence identity, to the amino acid sequence of SEQ ID NO. 33.

In some embodiments, the antigen-binding moiety comprises:
(4) a VL region incorporating the following CDRs.
   LC-CDR I having the amino acid sequence of SEQ ID NO 42
   LC-CDR2 having the amino acid sequence of SEQ ID NO 43
   LC-CDR3 having the amino acid sequence of SEQ ID NO 44,

or a variant thereof in which 1 or 2 or 3 amino acids in LC-CDR 1, and/or in which I or 2 or 3 amino acids in LC-CDR2, and/or in which 1 or 2 or 3 amino acids in LC-CDR3 are substituted with another amino acid.

In some embodiments, the antigen-binding moiety comprises.
(5) a VL region incorporating the following FRs:
   LC-FR1 having the amino acid sequence of SEQ ID NO:45
   LC-FR2 having the amino acid sequence of SEQ ID NO:46
   LC-FR3 having the amino acid sequence of SEQ ID NO:47
   LC-FR4 having the amino acid sequence of SEQ ID NO:48.
   or a variant thereof in which I or 2 or 3 amino acids in LC-FR1, and/or in which I or 2 or

3 amino acids in LC-FR2, and/or in which 1 or 2 or 3 ammo acids in LC-FR3, and/or in which 1 or 2 or 3 amino acids in LC-FR4 are substituted with another amino acid.

In some embodiments, the antigen-binding moiety comprises a VL region comprising the CDRs according to (4) and the FRs according to (5).

In some embodiments, the antigen-binding moiety comprises
(6) a VL region comprising an amino acid sequence having at least 70% sequence identity, more preferably one of at least 275%, ≥80%, ≥85%, ≥86%, ≥287%, ≥88%, ≥289%, ≥290%, ≥91%, ≥92%, ≥93%, ≥94%, ≥95%, ≥96%, ≥297%, ≥98%, ≥99% or ≥100%, sequence identity. to the amino acid sequence of SEQ ID NO:41.

In some embodiments, the antigen-binding moiety comprises a VH region according to any one of (1) to (3) above., and a VL. region according to any one of (4) to (6) above Substitutions of amino acids in accordance with the present disclosure may be biochemically conservative In some embodiments, where an amino acid to be substituted is provided in one of rows 1 to 5 of the table below. the replacement amino acid of the substitution is another. non-identical amino acid provided in the same row

| Row | Shared property | Amino acids |
|---|---|---|
| 1 | Hydrophobic | Met, Ala, Val, Leu, Ile, |
| | | Trp, Tyr, Phe, Norleucine |
| 2 | Neutral hydrophilic | Cys, Ser, Thr, Asn, Gln |
| 3 | Acidic or negatively- | Asp, Glu |
| | charged | |
| 4 | Basic or positively- | His, Lys, Arg |
| | charged | |
| 5 | Orientation influencing | Gly, Pro |

By way of illustration, in some embodiments wherein substitution is of a Met residue. the replacement amino acid may be selected from Ala, Val. Leu. Ile. Trp, Tyr. Phe and Norleucine.

In some embodiments, a replacement amino acid in a substitution may have the same side chain polarity as the amino acid residue it replaces. In some embodiments, a replacement amino acid in a substitution may have the same side chain charge (at pH 7 4) as the amino acid residue it replaces:

| | | Side-chain |
|---|---|---|
| Amino Acid | Side-chain polarity | charge (pH 7.4) |
| Alanine | nonpolar | neutral |
| Arginine | basic polar | positive |
| Asparagine | polar | neutral |
| Aspartic acid | acidic polar | negative |
| Cysteine | nonpolar | neutral |
| Glutamic acid | acidic polar | negative |
| Glutamine | polar | neutral |
| Glycine | nonpolar | neutral |
| Histidine | basic polar | positive (10%) |
| | | neutral (90%) |
| Isoleucine | nonpolar | neutral |
| Leucine | nonpolar | neutral |
| Lysine | basic polar | positive |
| Methionine | nonpolar | neutral |
| Phenylalanine | nonpolar | neutral |
| Proline | nonpolar | neutral |
| Serine | polar | neutral |
| Threonine | polar | neutral |
| Tryptophan | nonpolar | neutral |
| Tyrosine | polar | neutral |
| Valine | nonpolar | neutral |

That is, in some embodiments, a nonpolar amino acid is substituted with another., non-identical nonpolar amino acid In some embodiments, a polar amino acid is substituted with another, non-identical polar amino acid In some embodiments, an acidic polar amino acid is substituted with another, non-identical acidic polar amino acid. In some embodiments, a basic polar amino acid is substituted with another. non-identical basic polar amino acid In some embodiments, a neutral amino acid is substituted with another, non-identical neutral amino acid. In some embodiments, a positive amino acid is substituted with another, non-identical positive amino acid. In some embodiments, a negative amino acid is substituted with another, non-identical negative amino acid.

In some embodiments, substitution(s) may be functionally, conservative. That is, in some embodiments, the substitution may not affect (or may not substantially affect) one or more functional properties (e.g. target antigen binding) of the antigen-binding moiety comprising the substitution, as compared to the equivalent unsubstituted molecule.

In some embodiments, the sortase moiety is provided as a fusion polypeptide with a constituent polypeptide of the antigen-binding molecule. The sortase moiety may be connected to an amino acid sequence of an antigen-binding molecule via a linker sequence.

In some embodiments, the sortase moiety is fused to the N-terminus or the C-terminus of a constituent polypeptide of the antigen-binding molecule. In some embodiments, the sortase moiety is comprised in a polypeptide comprising a VH region (e.g a VH region according to an embodiment described hereinabove). In some embodiments, the sortase moiety is comprised in a polypeptide comprising a VL region (e.g a VL region according to an embodiment described hereinabove). In some embodiments, the sortase moiety is comprised in a polypeptide comprising a VH region and a VL region.

Immunoglobulins and their structures are described e.g in Schroeder and Cavacini J Allergy Clin Immunol. (2010) 125(20)2): S41-S52, which is hereby incorporated by reference in its entirety. Immunoglobulins of type G (i.e. IgG) are ˜150 kDa glycoproteins comprising two heavy chains and two light chains. From N- to C-terminus. immunoglobulin heavy chains comprise a VH follow ed by a heavy chain constant region comprising three constant domains (CH 1, CH2, and CH3, with a CH 1-CH2 hinge region provided between CH1 and CH2). Immunoglobulin light chains comprise a VL followed by a CL Depending on the heavy chain, immunoglobulins may be classed as IgG (e g. IgG1, IgG2, IgG3, IgG4), IgA (e.g IgA1, IgA2). IgD, IgE, or IgM. The light chain may be kappa (κ) or lambda (λ).

In some embodiments, the antigen-binding molecule of the present disclosure comprises one or more domains (e.g CH1, CH1-CH2 hinge, CH2. CH3, etc.) of an immunoglobulin heavy chain constant region sequence. In some embodiments, the immunoglobulin heavy chain constant region sequence is. or is derived from. the heavy chain constant region sequence of an IgG (e.g. IgG1, IgG2, IgG3, IgG4), IgA (e.g. IgA1, IgA2), IgD, IgE or IgM, e.g. a human IgG (e.g. higG1, hIgG2, hIgG3, hIgG4), higA (e.g hIgA1, hIgA2). hIgD, higE or hIgM. In some embodiments, the immunoglobulin heavy chain constant region sequence is, or is derived from. the heavy chain constant region sequence of a human IgG1 allotype (e.g. G1m1, G1m2, G1m3 or G1m17). In some embodiments, the antigen-binding molecule of the present disclosure comprises one or more domains of an immunoglobulin light chain constant region sequence. In some embodiments, the immunoglobulin light chain constant region sequence is human immunoglobulin kappa constant (IGKC, Cκ). In some embodiments, the immunoglobulin light chain constant region sequence is a human immunoglobulin lambda constant (IGLC: Cλ), e.g. IGLC1, IGLC2, IGLC3. IGLC6 or IGLC7.

In some embodiments, the sortase moiety is comprised in a polypeptide comprising one or more domains of an immunoglobulin constant region. Immunoglobulin heavy chain constant region domains include the CH1 region. the CH1 -CH2-hinge region. the CH2 region and the CH3 region. Immunoglobulin light chain constant region domains include the CL region.

Herein, a ‘CI-l region’ refers to an amino acid sequence corresponding to the CH1 region of an immunoglobulin. The CH1 region is the region of an Ig formed by positions 118 to 215 of the Ig constant region, according to the EU numbering system described in Edelman et al., Proc Nal Acad Sci USA (1969) 63(1): 78-85. A ‘CH 1-CI-12 hinge region’ refers to an amino acid sequence corresponding to the CH1-CH2 hinge region of an Immunoglobulin The CH1-CH2 hinge region is the region of an Ig formed by positions 216 to 230 of the Ig constant region, according to the EU numbering system described in Edelman et al., Proc Natd Acad Sci USA (1969) 63(1): 78-85. A ‘CH2 region’ refers to an amino acid sequence corresponding to the CH2 region of an Immunoglobulin. The CH2 region is the region of an Ig formed by positions 231 to 340 of the Ig constant region, according to the EU numbering system described in Edelman e al, Proc Nal Acad Sci USA (1969) 63(l): 78-85. A ‘CH3 region’ refers to an amino acid sequence corresponding to the CH3 region of an Immunoglobulin. The CH3 region is the region of an Ig formed by positions 341 to 447 of the Ig constant region, according to the EU numbering system described in Edelman ei al. Proc Natl Acad Sci USA (1969) 63(1): 78-85. A ‘CH2-CH3 region refers to an amino acid sequence corresponding to the -12 and CH3 regions of an Immunoglobulin. The CH2-CH3 region is the region of an Ig formed by positions 231 to 447 of the Ig constant region, according to the EU numbering system described in Edelman ei al, Proc Natl Acad Sci USA (1969) 63(1): 78-85.

In some embodiments, the antigen-binding molecule comprises a polypeptide comprising or consisting of one of the following structures:
N-term-[VI-I region]-[ . . . j-sortase moiety]-C-term
N-term-[VH region]-[CH1 region]-[ . . . ]-[sortase moiety]-C-term
N-term-IVH region]-[CH1 region-ICH1-CH2 hinge region]-[CH2 region]-[CH3 region]-[sortase moiety]-C-term
N-term-IVIL region]-[ . . . ]-[sortase moiety]-C-term
N-term-[VL region]-[CL region]-[..]-[sortase moiet]-C-term

In accordance with such embodiments, a ‘VH region’ may be a VH region as defined in one of (1) to (3) above, and a ‘VL region’ may be a VL region as defined in one of (4) to (6) above.

In some embodiments, an antigen-binding molecule of the present disclosure comprises a polypeptide that comprises. or consists of, an amino acid sequence having the amino acid sequence of SEQ ID NO. 29. or an amino acid sequence having at least 70% amino acid sequence identity, e.g. one of:275%, ≥80%, ≥85%, ≥90%, ≥91%, ≥92%, ≥93%, 94%, ≥95%, ≥96%, ≥97%, ≥98% or ≥99% sequence identity to SEQ ID NO:29. In some embodiments, the antigen-binding molecule comprises a polypeptide that comprises, or consists of, an amino acid sequence having the amino acid sequence of SEQ ID NO:30. or an amino acid sequence having at least 70% amino acid sequence identity, e.g one of ≥75%, ≥80%, ≥85%, ≥90% ≥91%, ≥92%, ≥93%, 94%, ≥95%, ≥96%, ≥97%, ≥98% or 99% sequence identity to SEQ ID NO: 30. In some 15 embodiments, the antigen-binding molecule comprises a polypeptide that comprises. or consists of, an amino acid sequence having the amino acid sequence of SEQ ID NO:31, or an amino acid sequence having at least 70% amino acid sequence identity. e.g one of ≥75%, ≥80%, ≥85%. ≥90%, ≥91%. ,92%, ≥93%, ≥94%, ≥95%, ≥96%, ≥97%, ≥98% or ≥99% sequence identity to SEQ ID NO:31. In some embodiments, the antigen-binding molecule comprises a polypeptide that comprises, or consists of, an amino acid sequence having the amino acid sequence of SEQ ID N0-32, or an amino acid sequence having at least 70% amino acid sequence identity. e.g one of ≥75%, ≥80%, ≥85%, ≥90%, ≥91%, ≥92%, ≥93%, ≥94%, ≥95%, ≥96%, ≥97%, ≥98% or ≥99% sequence identity to SEQ ID NO. 32.

In some embodiments, an antigen-binding molecule of the present disclosure comprises: (i) a polypeptide that comprises, or consists of, an amino acid sequence having the amino acid sequence of SEQ ID NO 29, or an amino acid sequence having at least 70% amino acid sequence identity, e.g. one of 75%, ≥80%, ≥85%, ≥90%, ≥91%, ≥92%, ≥93%, ≥94%, ≥95%, ≥96%. ≥97%, ≥98% or ≥99% sequence identity to SEQ ID NO:29, and (ii) a polypeptide that comprises, or consists of, an amino acid sequence having the amino acid sequence of SEQ ID NO 31, or an amino acid sequence having at least 70% amino acid sequence identity. e.g. one of 75%, ≥80%, ≥85%, ≥90%, ≥91%, ≥92%, ≥93%, ≥94%, ≥95%, ≥96%, ≥97%, ≥298% or ≥99% sequence identity to SEQ ID NO:31.

In some embodiments, an antigen-binding molecule of the present disclosure comprises: (i) a polypeptide that comprises, or consists of, an amino acid sequence having the amino acid sequence of SEQ ID NO:30, or an amino acid sequence having at least 70% amino acid sequence identity, e.g one of 75%, ≥8%, ≥85%, ≥90%, ≥91%, ≥92%, ≥93%, ≥94%, ≥295%. ¹96%, ≥97%, ≥98% or ≥99% sequence identity to SEQ ID NO:30, and (ii) a polypeptide that comprises, or consists of, an amino acid sequence having the amino acid sequence of SEQ I) NO:32, or an amino acid sequence having at least 70% amino acid sequence identity, e.g. one of 75%, ≥80%, ≥85%, ≥90%, 95, ≥92%, ≥93%, ≥94%, ≥95%, ≥296%, ≥97%, ≥98% or ≥99% sequence identity to SEQ ID NO. 32

### Detectable Moieties

In some aspects and embodiments, a polypeptide according to the present disclosure further comprises a detectable moiety. In some embodiments, a detectable moiety is provided at the N-terminus and/or C-terminus of the polypeptide

In accordance with such embodiments, it should be appreciated that the detectable moiety is non-identical to the identifier moiety of the polypeptide (or to be employed with the polypeptide).

The detectable moiety is useful for the identification and/or selection of T cells that have taken-up/initialised MHC molecules comprising the polypeptide of the present disclosure.

In some embodiments, a detectable moiety is a fluorescent label. phosphorescent label, luminescent label. immuno-detectable label (e g, an epitope tag), radiolabel. chemical. nucleic acid or enzymatic label. A polypeptide according to the present disclosure may be covalently or non-covalently labelled with the detectable moiety. In preferred embodiments, the detectable moiety is a fluorescent label.

Fluorescent labels include e.g fluorescein, rhodamine, allophycocyanin, eosine and NDB, green fluorescent protein (GFP). enhanced GFP (eFP), chelates of rare earths such as europium (Eu), terbium (Tb) and samanum (Sm), tetramethyl rhodamine. Texas Red, 4-methyl umbelliferone, 7-amino-4-methyl coumarin, Cy3, and Cy5. Radiolabels include radioisotopes such as Hydrogen³, Sulfur³⁵, Carbon¹⁴, Phosphorus³², lodine¹²³. Iodine¹²⁵, Iodine¹²⁶, Iodine¹³¹, Iodine¹³³, Bromine⁷⁷, Technetium^{99m}, Indium¹¹¹, Indium^{113m}, Gallium⁶⁷, Gallium⁶⁸, Ruthenium⁹⁵, Ruthenium⁹⁷, Ruthenium¹⁰³, Ruthenium¹⁰⁵, Mercury²⁰⁷, Mercury²⁰³, Rhenium^{99m}, Rhenium¹⁰¹, Rheniumie¹⁰⁵, Scandium⁴⁷, Tellurium^{121m}, Tellurium^{122m}, Telluriumm^{125m}, Thulium^{125m}, Thuliuml¹⁶⁵, Thulium¹⁶⁷, Copper⁶⁷, Fluorine¹⁸, Yttrium⁹⁰, Palladium¹⁰⁰, Bismutl²¹⁷ and Antimony²¹¹. Luminescent labels include as radioluminescent, chemiluminescent (e.g. acridinium ester, luminol, isoluminol) and bioluminescent labels. Immuno-detectable labels include haptens, peptides/polypeptides, antibodies, receptors and ligands such as biotin, avidin, streptavidin or digoxigenin. Nucleic acid labels include aptamers.

In some embodiments, the detectable moiety is an epitope tag, e.g. His. (e.g. 6XHis), FLAG. c-Myc, StrepTag. haemagglutinin, E. calmodulin-binding protein (CBP), glutathione-s-transferase (GST), maltose-binding protein (MBP), thioredoxmn, S-peptide, T7 peptide, SH2 domain, avidin, streptavidin, or hapten (e.g. biotin. digoxigenin. dinitrophenol).

In some embodiments, the detectable moiety is a moiety having a detectable activity e.g. an enzymatic moiety. Enzymatic moieties include e.g. luciferases, glucose oxidases, galactosidases (e.g. beta-galactosidase), glucuronidases, phosphatases (e.g. alkaline phosphatase), peroxidases (e.g. horseradish peroxidase) and cholinesterases.

### Further MHC Complex Polypeptide Sequences

In some embodiments, the polypeptide of the present disclosure further comprises an amino acid sequence capable of associating with the amino acid sequence of a MHC polypeptide as described hereinabove. for the formation of a MHC molecule.

That is, in some embodiments, the polypeptide comprises:
(i) the amino acid sequence of a major histocompatibility complex (MHC) polypeptide;
(ii) an amino acid sequence capable of associating with the amino acid sequence of (i) to form a MHC molecule; and
(iii) a moiety facilitating labelling of the polypeptide with an identifier moiety.

In some embodiments, the polypeptide comprises:
(i) the amino acid sequence of a major histocompatibility complex (MC-1 polypeptide,
(ii) an amino acid sequence capable of associating with the amino acid sequence of (i) to form a MHC molecule; and
(iii) an identifier moiety, wherein the identifier moiety is covalently associated with the polypeptide via linkage formed by a self-labelling protein a

In some embodiments, the polypeptide comprises:
(i) the amino acid sequence of a major histocompatibility complex (M-IC) polypeptide,
(ii) an amino acid sequence capable of associating with the amino acid sequence of (i) to form a MHC molecule;
(iii) a moiety facilitating labelling of the polypeptide with an identifier moiety; and
(iv) a sortase substrate motif.

In some embodiments, the polypeptide comprises:
(i) the amino acid sequence of a major histocompatibility complex (MHC) polypeptide:
(ii) an amino acid sequence capable of associating with the amino acid sequence of (i) to form a MHC molecule.
(iii) an identifier moiety. w herein the identifier moiety is covalently associated with the polypeptide via linkage formed by a self-labelling protein tag; and
(iv) a sortase substrate motif.

By way of illustration, the polypeptide of SEQ ID NO: 13 herein comprises the mature sequence of β2 microglobulin, and further comprises the amino acid sequence of mature I LA-A02 (connected via a flexible GS linker). The β2M and HLA-A02 sequences of the polypeptide associate to form a MHC molecule.

In embodiments wherein the polypeptide of the present disclosure comprises an amino acid sequence according to (ii) above, it will be appreciated that the amino acid sequence capable of associating with the amino acid sequence of a MHC polypeptide to form a MHC molecule is selected in accordance with the identity of the amino acid sequence of (i), such that the amino acid sequences are capable of association with one another to form a competent MHC molecule.

By way of illustration, in embodiments wherein the amino acid sequence of(i) is, or is derived from, the amino acid sequence of 2 microglobulin, the amino acid sequence of (ii) is, or is derived from, the amino acid sequence of an HLA class I u polypeptide (e g, an HLA-A, HLA-B, HLA-C, HLA-E, HLA-F or HLA-G polypeptide; e.g. an HLA-A, HLA-B or HLA-C polypeptide).

In some embodiments, the polypeptide of the present disclosure comprises.
(i) an amino acid sequence which is. or which is derived from, a MHC polypeptide selected from of Column A of Table 1:
(ii) an amino acid sequence which is, or which is derived from, a MHC polypeptide selected from of Column B of Table 1, and
(iii) a moiety facilitating labelling of the polypeptide with an identifier moiety.

In some embodiments, the polypeptide of the present disclosure comprises:
(i) an amino acid sequence which is. or which is derived from, a MHC polypeptide selected from of Column A of Table 1:
(ii) an amino acid sequence which is. or which is derived from, a MHC polypeptide selected from of Column B of Table 1 and
(iii) an identifier moiety. wherein the identifier moiety is covalently associated with the polypeptide via linkage formed by a self-labelling protein tag, wherein the MHC polypeptide selected from Column A of Table 1 and the MHC polypeptide selected from Column B of Table 1 are selected from the same row of Table 1.

In some embodiments, the polypeptide of the present disclosure comprises
(i) an amino acid sequence which is, or which is derived from, a MHC polypeptide selected from of Column A of Table 1;
(ii) an amino acid sequence which is. or which is derived from, a MHC polypeptide selected from of Column B of Table 1;
(iii) a moiety facilitating labelling of the polypeptide with an identifier moiety; and (iv) a sortase substrate motif.

In some embodiments, the polypeptide of the present disclosure comprises
(i) an amino acid sequence which is, or which is derived from, a MHC polypeptide selected from of Column A of Table 1;
(ii) an amino acid sequence which is. or which is derived from, a MHC polypeptide selected from of Column B of Table 1;
(iii) an identifier moiety. w herein the identifier moiety is covalently associated with the polypeptide via linkage formed by a self-labelling protein tag; and
(iv) a sortase substrate motif, wherein the MHC polypeptide selected from Column A of Table 1 and the MHC polypeptide selected from Column B of Table 1 are selected from the same row of Table 1.

TABLE 1

| Row | Column A | Column B |
|---|---|---|
| 1 | β2 microglobulin | A MHC class I α chain |
| | | polypeptide |
| 2 | β2 microglobulin | an HLA-A, HLA-B, HLA-C, |
| | | HLA-E, HLA-F or HLA-G |
| | | polypeptide |
| 3 | β2 microglobulin | an HLA-A, HLA-B or HLA-C |
| | | polypeptide |
| 4 | β2 microglobulin | an HLA-A polypeptide (e.g. |
| | | an HLA-A02, HLA-A01, |
| | | HLA-A03, HLA-A11 or |
| | | HLA-A24 polypeptide) |
| 5 | A MHC class I α chain | β2 microglobulin |
| | polypeptide | |
| 6 | an HLA-A. HLA-B, HLA-C, | β2 microglobulin |
| | HLA-E, HLA-F or HLA-G | |
| | polypeptide | |
| 7 | an HLA-A, HLA-B or HLA-C | β2 microglobulin |
| | polypeptide | |
| 8 | an HLA-A polypeptide (e.g. | β2 microglobulin |
| | an HLA-A02, HLA-A01, HLA- | |
| | A03, HLA-A11 or HLA-A24 | |
| | polypeptide) | |
| 9 | A MHC class II α chain | A MHC class II β chain |
| | polypeptide | polypeptide |
| 10 | an HLA-DPA1, HLA-DQA1, | an HLA-DPB1, HLA-DQB1, |
| | HLA-DQA2 or HLA-DRA | HLA-DQB2, HLA-DRB1, |
| | polypeptide | HLA-DRB3, HLA-DRB4 or |
| | | HLA-DRB5 polypeptide |
| 11 | A MHC class II β chain | A MHC class II α chain |
| | polypeptide | polypeptide |
| 12 | an HLA-DPB1, HLA-DQB1, | an HLA-DPA1, HLA-DQA1, |
| | HLA-DQB2, HLA-DRB1, | HLA-DQA2 or HLA-DRA |
| | HLA-DRB3, HLA-DRB4 or | polypeptide |
| | HLA-DRB5 polypeptide | |

### Linkers, Labels and Conjugates

The polypeptides of the present disclosure may additionally comprise further amino acids or sequences of amino acids.

The polypeptides may comprise one or more linker sequences between sequences of amino acids. By way of example, a linker sequence may be provided between different domains of a polypeptide (e.g. between the amino acid sequence of a MHC polypeptide and the moiety facilitating labelling of the polypeptide with an identifier moiety).

Linker sequences are known to the skilled person, and are described, for example in Chen et al.. Adv Drug Deliv Rev (2013) 65(10): 1357-1369, which is hereby incorporated by reference in its entirety. In some embodiments, a linker sequence may be a flexible linker sequence. Flexible linker sequences allow for relative movement of the amino acid sequences which are linked by the linker sequence. Flexible linkers are known to the skilled person, and several are identified in Chen c al. Adv Drug Deli Rev, (2013) 65(10): 1357-1369. Flexible linker sequences often comprise high proportions of glycine and/or serine residues.

In some embodiments, a linker sequence comprises at least one glycine residue and/or at least one serine residue. In some embodiments, the linker sequence comprises or consists of glycine and serine residues In some embodiments., the linker sequence has the structure (GxS)n or (GxS)nGm, wherein G=glycine. S=serine, x=3 or 4, n=2, 3. 4. 5 or 6, and m=0, 1. 2 or 3.

In some embodiments, the linker sequence comprises one or more (e.g. 1. 2, 3, 4, 5 or 6) copies (e.g in tandem) of the sequence motif G₄S. In some embodiments, a linker sequence comprises or consists of (G₄S)₃ or (G₄S )₄. In some embodiments, the linker sequence has a length of 1-2. 1-3. 1-4, 1-5. 1-10, 1-15, 1-20, 1-25. or 1-30 amino acids.

In some embodiments, the linker sequence comprises one or more copies of an amino acid sequence according to SEQ ID NO:7 or 8 In some embodiments, the linker sequence comprises at least 1. 2. 3 or 4 copies of an amino acid sequence according to SEQ ID NO:6.

In some embodiments, the linker sequence comprises. or consists of, an amino acid sequence having at least 60%, preferably one of 70%, ≥75%, ≥80% ≥85%, ≥90%, ≥91%, ≥92%, ≥93%, ≥94%, ≥95%, 96%, ≥97%, ≥98%, ≥99% or ≥100% amino acid sequence identity to SEQ ID NO. 7 or 8.

The polypeptides of the present disclosure may comprise amino acid sequence(s) to facilitate expression, folding. trafficking. processing. purification or detection of the antigen-binding molecule/polypeptide. For example, polypeptides of the present disclosure may additionally comprise a sequence of amino acids forming a detectable moiety, e.g. as described hereinabove.

The polypeptides may additionally comprise a signal peptide (also known as a leader sequence or signal sequence). Signal peptides nominally consist of a sequence of 5-30 hydrophobic amino acids, which form a single alpha helix. Secreted proteins and proteins expressed at the cell surface often comprise signal peptides. Signal peptides are known for many proteins, and are recorded in databases such as GenBank. UniProt and Ensembl, and/or can be identified/predicted e.g using amino acid sequence analysis tools such as SignalP (Petersen et al, 2011 Nature Methods 8 785-786) or Signal-BLAST (Frank and Sippl. 2008 Bioinformatics 24. 2172-2176).

The signal peptide may be present at the N-terminus of the polypeptide, and may be present in the newly-synthesised polypeptide. The signal peptide provides for efficient trafficking of the polypeptide Signal peptides are often removed by cleavage, and thus are not comprised in the mature polypeptide.

Signal peptides are known for many proteins, and are recorded in databases such as GenBank. UniProt, Swiss-Prot, TrEMBL, Protein Information Resource. Protein Data Bank. Ensembl, and InterPro, and/or can be identified/predicted e.g. using amino acid sequence analysis tools such as SignalP (Petersen et al., 2011 Nature Methods 8. 785-786) or Signal-BLAST (Frank and Sippl, 2008 Bioinformatics 24: 2172-2176).

In some embodiments, the signal peptide comprises. or consists of, an amino acid sequence having at least 60%. preferably one of 0.70%, ≥75%, ≥80%, ≥85%, ≥90%, ≥91%, ≥92%, ≥93%, 94%, ≥9%, ≥96%, ≥97%, ≥98%, ≥99% or ≥100% amino acid sequence identity to SEQ ID NO: 2.

### Particular Exemplary Polypeptides, MHC Molecules and MHC:Peptide Complexes

In some embodiments, a polypeptide according to the present disclosure comprises or consists of one of the following structures

N-term-[signal peptide]-[moiety facilitating labelling of the polypeptide with an identifier moiety]-[amino acid sequence of [a MHC polypeptide]-C-term

N-term-[moiety facilitating labelling of the polypeptide with an identifier moiety]-[amino acid sequence of a MHC polypeptide]-C-term

N-term-[signal peptide-IfHaloTage]-[mature β2 microglobulin sequence]-C-tern

N-term-[HaloTag®]-[mature β2 nucroglobulin sequence]-C-term

N-term-[signal peptide]-[moiety facilitating labelling of the polypeptide with an identifier moiety]-[amino acid sequence of a MHC polypeptide]-[amino acid sequence capable of associating with the amino acid sequence of a MHC polypeptide to form a MHC molecule]-C-term

N-term-[moiety facilitating labelling of the polypeptide with an identifier moiety]-[amino acid sequence of a MHC polypeptide]-[amino acid sequence capable of associating with the amino acid sequence of a MHC polypeptide to form a MHC molecule]-C-term

N-term-[signal peptide]-[HaloTag® ]-[mature β2 microglobulin sequence]-[mature HLA-A02 sequence]-C-ter.

N-term-[signal peptide]-[HaloTag®]W-[tmature β2 microglobulin sequence]-[mature HLA class I a polypeptide sequence]-C-term

N-term-[HaloTag;]-[mature [32 inicroglobulin sequence]-[mature HLA class I a polypeptide sequence]-C-term

N-term-[signal peptide]-[moiety facilitating labelling of the polypeptide with an identifier moiety -[sortase substrate motif]-[amino acid sequence of a protein that localises to the cell membrane]-C-term

N-term-[noiets facilitating labelling of the polypeptide with an identifier moiety]-[sortase substrate motif]-[amino acid sequence of a protein that localises to the cell membrane]-C-term

N-term-[signal peptide]-[moiety facilitating labelling of the polypeptide with an identifier moiety 1-[sortase substrate motif]-[amino acid sequence of a MHC polypeptide]-C-term

N-term-[moiety facilitating labelling of the polypeptide with an identifier moiety]-[sortase substrate motif]-[amino acid sequence of a MHC polypeptide]-C-term

N-term-[signal peptide]-|HaloTag®;]-[sortase substrate motitf-[mature β2 microglobulin sequence[-C-term

N-term-[HaloTag®]-[sortase substrate motif]-[mature 132 microglobulin sequence]-C-term

N-term-[signal peptide]-[moiety facilitating labelling of the polypeptide with an identifier moiety]-[sortase substrate motif]-[amino acid sequence of a MHC polypeptide]-[amino acid sequence capable of associating with the amino acid sequence of a MHC polypeptide to form a MHC molecule]-C-term

N-term-[moiety facilitating labelling of the polypeptide with an identifier moiety]-[sortase substrate motif]-[amino acid sequence of a MHC polypeptide]-[amino acid sequence capable of associating with the amino acid sequence of a MHC polypeptide to form a MHC molecule]-C-term.

N-term-[signal peptide]-[HaloTag” @W-[sortase substrate motif]-[mature [ ]2 microglobulin sequence]-[mature HL A-A02 sequence]-C-term

N-term-[signal peptide]-[HaloTag®]-[sortase substrate motif]-[mature β2 microglobulin sequence]-[mature HLA class I a. polypeptide sequence]-C-term

N-term-[HaloTag]-[sortase substrate motif]-[mature [2 microglobulin sequence]-[mature HLA class I α polypeptide sequence]-C-term The present disclosure further provides:
(1) A polypeptide comprising or consisting of (e.g. from N-terminus to C-terminus):
   (i) an amino acid sequence encoding a signal peptide. e.g. an amino acid sequence having at least 70%, preferably one of ≥80%, ≥85%, ≥90%, ≥91%, ≥92%, ≥93%, ≥94%, ≥95%, ≥296%, 0.97%, ≥98%, ≥99% or ≥0x % amino acid sequence identity. to SEQ ID NO:2:
   (ii) an amino acid sequence encoding a moiety facilitating labelling of the polypeptide with an identifier moiety (e.g. a self-labelling protein tag. e.g. a HaloTag K): e.g. an amino acid sequence having at least 70%, preferably one of ≥80%, ≥85%, ≥90%, ≥91%, ≥92%, ≥93%, 0.94%, ≥95%,6, % 97%, ≥98%, ≥99% or ≥100% amino acid sequence identity, to SEQ ID NO. 4 or 5; and
   (iii) an amino acid sequence encoding the amino acid sequence of a MHC polypeptide (e.g. β2 microglobulin); e.g. an amino acid sequence having at least 70%. preferably one of 0.280%, ≥85%, ≥90%, ≥91%, ≥292%, 93%, ≥94%, ≥95%, ≥96%, ≥97%, ≥98%, ≥99% or ≥100% amino acid sequence identity. to SEQ ID NO. 3.
(2) A polypeptide comprising or consisting of (e g. from N-terminus to C-terminus):
   (i) an amino acid sequence encoding a moiety facilitating labelling of the polypeptide with an identifier moiety (e.g. a self-labelling protein tag. e.g a HaloTag®; e.g. an amino acid sequence having at least 70%. preferably one of ≥80%, ≥85%, ≥90%, ≥91%, ≥92%, ≥93%. ≥94%, ≥95%., ≥96%, ≥97%, ≥98%, ≥99% or ≥100% amino acid sequence identity, to SEQ ID NO:4 or 5, and
   (ii) an amino acid sequence encoding the amino acid sequence of a MHC polypeptide (e.g β2 microglobulin): e.g. an amino acid sequence having at least 70%, preferably one of ≥80%, ≥85% ≥90%, ≥91%, ≥92%, ≥93%, ≥94%, ≥95%:96%, ≥97% ≥98%, ≥99% or ≥100% amino acid sequence identity, to SEQ ID NO:3.
(3) A polypeptide comprising or consisting of(e.g. from N-terminus to C-terminus): (i) an amino acid sequence encoding a signal peptide. e.g an amino acid sequence having at least 70%. preferably one of 80%, ≥85%, ≥90%, ≥91%, ≥92%., 93%, ≥94%, ≥95%, ≥96%. =97%, ≥98%, ≥99% or ≥100% amino acid sequence identity, to SEQ ID NO::2;
(ii) an amino acid sequence encoding a moiety facilitating labelling of the polypeptide with an identifier moiety (e.g. a self-labelling protein tag. e.g a HaloTag®) e.g an amino acid sequence having at least 70%. preferably one of 80%., 85%, ≥90%, ≥91%, ≥92%, ≥93%, 94%, ≥95%, ≥96%, ≥97%, ≥8%, ≥99% or 100% amino acid sequence identity. to SEQ ID NO 4 or 5.
(iii) an amino acid sequence encoding the amino acid sequence of β2 microglobulin; e.g. an amino acid sequence having at least 70%. preferably one of 80%, ≥85%, ≥90%, ≥91%, ≥92%, ≥93%, ≥94%, ≥95%, ≥96%, ≥97%, ≥98%, ≥99% or 100% amino acid sequence identity, to SEQ ID NO:3; and
(iv) an amino acid sequence encoding the amino acid sequence of an HLA class I a polypeptide; e.g. an amino acid sequence having at least 70%. preferably one of =,80%, ≥285%. 90%, ≥91%, ≥92%, ≥93%, ≥94%, ≥95%, ≥96%, ≥97%, ≥98%, ≥99% or ≥100% amino acid sequence identity, to SEQ ID NO: 9.
(4) A polypeptide comprising or consisting of (e.g. from N-terminus to C-terminus):
   (i) an amino acid sequence encoding a moiety facilitating labelling of the polypeptide with an identifier moiety (e.g. a self-labelling protein tag. e.g a HaloTag): e.g an amino acid sequence having at least 70%. preferably one of 80%, ≥85%, ≥90%, ≥91%, ≥92%, ≥93%, 94%, ≥95%, ≥96%, ≥97%, ≥98%, ≥99% or ≥100% amino acid sequence identity, to SEQ ID NO 4 or 5.
   (ii) an amino acid sequence encoding the amino acid sequence of β2 nucroglobulin: e.g an amino acid sequence having at least 70%, preferably one of 80%, ≥85%, ≥90%, ≥91%, ≥92%, ≥93%, ≥94%, ≥95%, ≥96%, ≥97%, ≥98%, ≥99% or 100% amino acid sequence identity, to SEQ ID NO; and
   (iii) an amino acid sequence encoding the amino acid sequence of an HLA class I a polypeptide; e.g. an amino acid sequence having at least 70%. preferably one of =,80%, ≥285%, ≥90%, ≥291%, ≥92%, ≥93%, ≥94%, ≥95%, ≥96%, ≥97%, ≥98%, ≥99% or ≥100% amino acid sequence identity, to SEQ ID NO: 9.
(5) A polypeptide according to the present disclosure comprises or consists of an amino acid sequence having at least 70%, preferably one of ≥80%, ≥85%, ≥90% ≥91%, =92%, ≥93%, 94%, ≥95%, ≥96%, ≥97%, ≥98%, ≥99% or ≥100% amino acid sequence identity. to SEQ ID NO 10.
(6) A polypeptide according to the present disclosure comprises or consists of an amino acid sequence having at least 70%, preferably one of ≥80%, ≥85%, ≥290%, ≥91% ≥93%. 094%, ≥9%, ≥96%, ≥97%, ≥98%, ≥99% or ≥100% amino acid sequence identity, to SEQ ID NO: 11.
(7) A polypeptide according to the present disclosure comprises or consists of an amino acid sequence having at least 70%, preferably one of 80%, ≥85%, ≥90%, ≥91%, ≥92%, ≥93%, ≥94%, ≥95%, ≥96%, ≥97, ≥98%, ≥99% or ≥100% amino acid sequence identity. to SEQ ID NO. 12.
(8) A polypeptide according to the present disclosure comprises or consists of an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or ≥100% amino acid sequence identity. to SEQ ID NO 13.
(9) A polypeptide comprising or consisting of (e.g. from N-terminus to C-terminus):
   (i) an amino acid sequence encoding a signal peptide. e.g. an amino acid sequence having at least 70%, preferably one of ≥80%, ≥85%, ≥90%, ≥91%, ≥92%, ≥93%, ≥94%, ≥95%, ≥96%, ≥97%, ≥98%, ≥99% or ≥100% amino acid sequence identity. to SEQ ID NO:2:
   (ii) an amino acid sequence encoding a moiety facilitating labelling of the polypeptide with an identifier moiety (e.g. a self-labelling protein tag. e.g. a HaloTag ): e.g. an amino acid 25 sequence having at least 70%, preferably one of ≥80%, ≥85%, ≥90%, ≥91%, ≥92%, ≥93%, ≥94%, ≥95%, ≥96%, ≥97%, ≥98%, ≥99% or ≥100)% amino acid sequence identity, to SEQ ID NO. 4 or 5:
   (iii) an amino acid sequence conforming to the consensus of SEQ ID NO: 25; and
   (iv) an amino acid sequence encoding the amino acid sequence of a MHC polypeptide (e.g. β2 microglobulin); e.g. an amino acid sequence having at least 70%, preferably one of 280%, ≥85%, ≥90%, ≥91%, ≥92%, ≥93%, ≥94%, ≥95%, ≥96%, ≥97%, ≥98%, ≥99% or ≥100% amino acid sequence identity, to SEQ ID NO:3.
(10) A polypeptide comprising or consisting of (e.g. from N-terminus to C-terminus):
   (i) an amino acid sequence encoding a moiety facilitating labelling of the polypeptide with an identifier moiety (e.g a self-labelling protein tag, e g, a HaloTag): e.g. an amino acid sequence having at least 70%, preferably one of ≥8% ≥85%, ≥90%, ≥91%, ≥92%, ≥93%, ≥94%, ≥95%, ≥96%, ≥97, ≥98%, ≥99% or ≥100% amino acid sequence identity. to SEQ ID 5 NO. 4 or 5:
   (ii) an amino acid sequence conforming to the consensus of SEQ ID NO:25:
   (iii) an amino acid sequence encoding the amino acid sequence of a MHC polypeptide (e.g. D2 microglobulin); e.g. an amino acid sequence having at least 70%, preferably one of 80%, ≥85%, ≥90%, ≥91%, ≥92%, ≥93%, ≥94%, ≥95%, ≥96%, ≥97% ≥98%, ≥99% or ≥1001% amino acid sequence identity, to SEQ ID NO:3.
(11) A polypeptide comprising or consisting of (e.g. from N-terminus to C-terminus):
   (i) an amino acid sequence encoding a signal peptide, e g, an amino acid sequence having at least 70%, preferably one of ≥80%, ≥85%, ≥90%, ≥91%, ≥92%, ≥93%, ≥94%, ≥95%, ≥96%, =97%, ≥98%, ≥99% or ≥100% amino acid sequence identity, to SEQ ID NO:2;
   (ii) an amino acid sequence conforming to the consensus of SEQ ID NO:25.
   (iii) an amino acid sequence encoding a moiety facilitating labelling of the polypeptide with an identifier moiety (e.g. a self-labelling protein tag, e g, a HaloTag® ): e.g. an amino acid sequence having at least 7(0%, preferably one of ≥80%, ≥85%, ≥90%, ≥91%, ≥92%, ≥93%, ≥94%, ≥95%, ≥96%, ≥97%. =98%, ≥99% or ≥1000% amino acid sequence identity. to SEQ ID NO:4 or 5:
   (iv) an amino acid sequence encoding the amino acid sequence of β2 microglobulin: e.g an amino acid sequence having at least 70%, preferably one of 80%, ≥85% ≥90% ≥91%, ≥92%, ≥93%, ≥94%, ≥95%, ≥96%, ≥97%, ≥98%, ≥99% or ≥100% amino acid sequence identity , to SEQ ID NO:3; and
(v) an amino acid sequence encoding the amino acid sequence of an HLA class I a polypeptide. e.g. an amino acid sequence having at least 70%. preferably one of 80%, ≥85%, 90%, ≥91%, ≥92%, ≥93%, ≥94%, ≥95%, ≥96%, ≥97%, ≥98%, ≥99% or ≥100% amino acid sequence identity, to SEQ ID NO:9.
(12) A polypeptide comprising or consisting of(e.g. from N-terminus to C-terminus):
   (i) an amino acid sequence encoding a moiety facilitating labelling of the polypeptide with an identifier moiety (e.g. a self-labelling protein tag, e g, a HaloTag® ); e.g. an amino acid sequence having at least 7% %, preferably one of ≥80%, ≥85%, ≥90%, ≥91%, ≥92%, ≥93%, ≥94%, ≥95%, ≥96%, 97%, ≥98% ≥99% or 100% amino acid sequence identity. to SEQ ID NO 4 or 5:
   (ii) an amino acid sequence conforming to the consensus of SEQ ID NO:25:
   (iii) an amino acid sequence encoding the amino acid sequence of 12 microglobulin; e.g. an amino acid sequence having at least 70%. preferably one of 80%, ≥85%, ≥90%, ≥291%. ≥92%, ≥93%, ≥94%, ≥95%, ≥96%, ≥97%, ≥98%, ≥99% or ≥100% amino acid sequence identity, to SEQ ID NO:3; and
(iv) an amino acid sequence encoding the amino acid sequence of an HLA class I α polypeptide: e.g. an amino acid sequence having at least 70%., preferably one of ≥80%, ≥85%,90% ≥91%, ≥92%, ≥93%, ≥94%, ≥95%, ≥96%, ≥97%, ≥98%, ≥99% or ≥100% amino acid sequence identity. to SEQ ID NO:9.

The present disclosure further provides:
(A) A MHC molecule comprising or consisting of: (i) α polypeptide according to (2) or (6) above, and (ii) a MHC polypeptide capable of associating the polypeptide of (i) to form a MHC molecule; e.g. an 1-ILA class I a polypeptide.
(B) A MHC molecule comprising or consisting of α polypeptide according to (4) or (8) above.
(C) A MHC molecule comprising or consisting of. (i) α polypeptide according to (10) above, and (ii) a MHC polypeptide capable of associating with the polypeptide of (i) to form a MHC molecule: e.g an HLA class I α polypeptide.
(D) A MHC molecule comprising or consisting of α polypeptide according to (12) above.

The present disclosure further provides:
(I) A MHC:peptide complex comprising a MHC molecule according to (A) or (B) above. and a peptide presented by the MHC molecule.
(II) A MHC:peptide complex comprising. (i) a MHC molecule according to (A) or (B) above. (ii) a peptide presented by the MHC molecule, and (ii) an identifier moiety (e.g. a nucleic acid identifier moiety, e g, an ssDNA identifier moiety),
(III) A MHC:peptide complex comprising a MHC molecule according to (C) or (D) above. and a peptide presented by the MHC molecule.
(IV) A MHC-peptide complex comprising. (i) a MHC molecule according to (C) or (D) above. (ii) a peptide presented by the MHC molecule, and (ii) an identifier moiety (e.g. a nucleic acid identifier moiety. e.g. an ssDNA identifier moiety).

### Nucleic Acids and Vectors

The present disclosure provides nucleic acids, or pluralities of nucleic acids, encoding polypeptides according to the present disclosure (e.g. α polypeptide or antigen-binding molecule according to any aspect or embodiment described herein). In some embodiments, the nucleic acid(s) comprise or consist of DNA and/or RNA.

A polypeptide according to the present disclosure may be produced within a cell by translation of RNA encoding the polypeptide A polypeptide according to the present disclosure may be produced within a cell by transcription from nucleic acid encoding the polypeptide, and subsequent translation of the transcribed RNA.

In some embodiments, the nucleic acid(s) may be, or may be comprised/contained in, a vector, or a plurality of vectors. A ‘vector’ as used herein is a nucleic acid molecule used as a Vehicle to transfer exogenous nucleic acid into a cell.

Accordingly. the present disclosure also provides a vector, or plurality of vectors, comprising the nucleic acid or plurality of nucleic acids according to the present disclosure. The vector may facilitate delivery of the nucleic acid(s) encoding a polypeptide according to the present disclosure to a cell The vector may be an expression vector comprising elements required for expressing α polypeptide according to the present disclosure. The vector may comprise elements facilitating integration of the nucleic acid(s) into the genomic DNA of cell into which the vector is introduced.

Nucleic acids and vectors according to the present disclosure may be provided in purified or isolated form, i.e. from other nucleic acid, or naturally -occurring biological material.

A vector may be a vector for expression of the nucleic acid in the cell (i.e. an expression vector) Such vectors may include a promoter sequence operably linked to a nucleotide sequence encoding a polypeptide according to the present disclosure. A vector may also include a termination codon (i.e. 3′ in the nucleotide sequence of the vector to the nucleotide sequence encoding the polypeptide) and expression enhancers Any suitable vectors, promoters, enhancers and termination codons known in the art may be used to express a peptide or polypeptide from a vector according to the present disclosure.

The term ‘operably linked’ may include the situation where nucleic acid encoding a polypeptide according to the present disclosure and regulatory nucleic acid sequence(s) (e.g a promoter and/or enhancers) are covalent linked in such a way as to place the expression of the nucleic acid encoding a polypeptide under the influence or control of the regulatory nucleic acid sequence(s) (thereby forming an expression cassette). Thus, a regulatory sequence is operably linked to the selected nucleic acid sequence if the regulatory sequence is capable of effecting transcription of the nucleic acid sequence. The resulting transcript(s) may then be translated into the desired polypeptide(s).

Vectors contemplated in connection with the present disclosure include DNA vectors. RNA vectors, plasmids (e.g. conjugative plasmids (e.g. F plasmids). non-conjugative plasmids, R plasmids. col plasmids. episomes), viral vectors (e.g. retroviral vectors. e.g. gammaretroviral vectors (e g. murine Leukemia virus (MLV)-derived vectors, e.g. SFG vector), lentiviral vectors, adenovirus vectors, adeno-associated virus vectors. vaccinia virus vectors and herpesvirus vectors), transposon-based vectors, and artificial chromosomes (e.g. yeast artificial chromosomes). e.g. as described in Maus et al., Annu Rev Immunol (2014) 32.189-225 and Morgan and Boyerinas, Biomedicines (2016) 4:9. which are both hereby incorporated by reference in their entirety. In some embodiments, a vector according to the present disclosure is a lentiviral vector.

In some embodiments, the vector may be a eukaryotic vector, i e a vector comprising the elements necessary for expression of protein from the vector in a eukaryotic cell. In some embodiments, the vector may be a mammalian vector. e.g. comprising a cytomegalovirus (CMV) or SV40 promoter to drive protein expression.

### Peptides

The articles and methods of the present disclosure find use in the identification of T cell receptors (TCRs) capable of binding to MHC-peptide complexes. In particular. it is contemplated to employ the articles and methods of the present disclosure for the identification of TCRs capable of binding to MI C molecules presenting peptide(s) of interest.

A peptide of the present disclosure may be a peptide of α polypeptide of interest A peptide of a reference polypeptide of interest may be any fragment of the polypeptide.

A polypeptide of interest may be any polypeptide. A polypeptide of interest may for example be α polypeptide. or α polypeptide of an infection agent or cell. to which it is desired to direct an immune response (e g, a cell-mediated immune response). A polypeptide of interest may be a disease-associated antigen.

A ‘disease-associated antigen’ refers to an antigen whose presence is indicative of a given disease/disease state. or an antigen for which an elevated level of the antigen is positively-correlated with a given disease/disease state. The disease-associated antigen may be an antigen whose expression is associated with the development. progression or severity of symptoms of a given disease. The disease-associated antigen may be associated with the cause or pathology of the disease, or may be expressed abnormally as a consequence of the disease A disease-associated antigen may be an antigen of an infectious agent or pathogen, a cancer-associated antigen or an autoimmune disease-associated antigen In some embodiments, the disease-associated antigen is an antigen of a pathogen. The pathogen may be prokaryotic (bacteria), eukaryotic (e.g. protozoan, helminth, fungus). virus or pron.

In some embodiments, the pathogen is an intracellular pathogen. In some embodiments the pathogen is a virus. A virus may be a dsDNA virus (e.g adenovirus. herpes virus, poxvirus), ssRNA virus (e.g. parvovirus), dsRNA virus (e.g. reovirus), (+”)ssRNA virus (e.g. picornavirus, togavirus). (-)ssRNA virus (e.g. orthomyxovirus. rhabdovirus), ssRNA-RT virus (e.g. retrovirus) or dsDNA-RT virus (e.g. hepadnavirus). In particular, viruses of the families adenoviridae, herpesviridae, papillomaviridae, polyomaviridae, poxviridae, hepadnaviridae, parvoviridae, astroviridae, caliciviridae, picornaviridae, coronaviridae, flaviviridae, togaviridae, Hepeviridae. retroviridae. orthomyxoviridae, arenavindae. bunvaviridae, filovindae, paramyxoviridae, rhabdoviridae and reoviridae are contemplated. In some embodiments the virus is selected from Epstein-Barn virus, adenovirus, herpes simplex type I virus. herpes simplex type 2 virus. Varicella-Zoster virus, cytomegalovirus. human herpes virus type 8, human papillomavirus, BK virus. JC virus, smallpox. hepatitis B virus, Parvovirus β139, human astrovirus, lymphocytic choriomeningitis virus, Norwalk virus, coxsackievirus, hepatitis A virus, poliovirus, rhinovirus, severe acute respiratory syndrome virus, hepatitis C virus, yellow fever virus, dengue virus, West Nile virus. TBE virus, rubella virus. hepatitis E virus, human immunodeficiency virus, influenza virus. lassa virus, Crimean-Congo hemorrhagic fever virus. Hantaan virus. ebola virus. Marburg virus. measles virus, mumps virus, parainfluenza virus, picornavirus, respiratory syncytial virus, rabies virus, hepatitis D virus, rotavirus, orbivirus, coltivirus, and banna virus. In some embodiments, the pathogen is a bacterium The bacterium may be gram-positive or grain-negative. In particular, bacteria of the genera Bacillus, Bartonella. Bordetella, Borrelia. Brucella, Campylobacter, Chlamvdia, Chlamydophila, Clostridium, Corvnebacterium. Enterococcus, Escherichia. Francisella. Haemophilus, Helicobacter. Legionella. Leptospira. Listeria. Mycobacterium, Mycoplasma Neisseria, Pseudomonas. Rickettsia. Salmonella, Shigella. Staphylococcus. Streptococcus. Treponema, Ureaplasma. Vibrio and Yersinia are contemplated. In some embodiments the pathogen is protozoan. In particular. protozoa of the genera Entamoeba. Plasmodium, Giardia, Trypanosoma. Leishmania, Besnoitia and Toxoplasma are contemplated. In some embodiments the pathogen is a fungus. In particular, fungi of the genera Candida, Aspergillus, Blastomyces, Coccidioides. Sporothrix. Cryptococcus. Histoplasma, Pneumocystis, Stachy botrys. Rhizopus. Mucor, Cunninghamella, Apophysomyces, Trichophy ton. Microsporum. Epidennophyton, Fusarium, and Lichtheimia are contemplated.

In some embodiments, a polypeptide of interest is a polypeptide whose expression/activity. or whose upregulated expression/activity, is positively-associated with a disease or disorder (e.g. a cancer, an infectious disease or an autoimmune disease). In some embodiments the polypeptide of interest is associated with a cancer, an infectious disease. or an autoimmune disease.

In some embodiments, the disease-associated antigen is a cancer cell antigen. A cancer cell antigen is an antigen which is expressed or over-expressed by a cancer cell. A cancer cell antigen may be any peptide./polypeptide, glycoprotein. lipoprotein. glycan, glycolipid. lipid, or fragment thereof. A cancer cell antigen's expression may be associated with a cancer. A cancer cell antigen may be abnormally expressed by a cancer cell (e g. the cancer cell antigen may be expressed with abnormal localisation), or may be expressed with an abnormal structure by a cancer cell A cancer cell antigen may be capable of eliciting an immune response. The cancer cell antigen may be a cancer-associated antigen. In some embodiments the cancer cell antigen is an antigen w hose expression is associated with the development, progression or severity of symptoms of a cancer. The cancer-associated antigen may be associated with the cause or pathology of the cancer, or may be expressed abnormally as a consequence of the cancer. In some embodiments, the cancer cell antigen is an antigen whose expression is upregulated (e g, at the RNA and/or protein level) by cells of a cancer, e.g. as compared to the level of expression of by comparable non-cancerous cells (e.g. non-cancerous cells derived from the same tissue/cell type). In some embodiments, the cancer-associated antigen may be preferentially expressed by cancerous cells, and not expressed by comparable non-cancerous cells (e g. non-cancerous cells derived from the same tissue/cell ty pe). In some embodiments, the cancer-associated antigen may be the product of a mutated oncogene or mutated tumor suppressor gene. In some embodiments, the polypeptide of interest is a cancer-associated neoantigen. In some embodiments, the cancer-associated antigen may be the product of an overexpressed cellular protein, a cancer antigen produced by an oncogenic virus, an oncofetal antigen, or a cell surface glycolipid or glycoprotein.

It will be appreciated that in preferred embodiments, a peptide according to the present disclosure is a peptide capable of participating in a MHC:peptide complex. That is, the peptide is capable of associating with a MHC molecule according to the present disclosure (i.e. a MHC molecule comprising a polypeptide according to the present disclosure) to form a MHC:peptide complex

In some embodiments, the peptide has a length of 5 to 30, e.g. 10 to 25 or 8 to 11 amino acids.

### MHC Molecules and MHC:Peptide Complexes

The present disclosure provides MHC molecules comprising a polypeptide according to the present disclosure The MHC molecule may be a MHC class I molecule or a MHC class II molecule. In preferred embodiments, the MHC molecule is a MHC class 1 molecule.

The MHC molecule is formed of α polypeptide complex, formed by association between a polypeptide according to the present disclosure and another MHC polypeptide It will be appreciated that the constituent polypeptides of the MHC molecule are selected so as to form a competent MHC molecule. By way of illustration, in embodiments wherein the polypeptide of the present disclosure comprises the amino acid sequence of β2 microglobulin, the other MHC polypeptide of a MHC molecule comprising the polypeptide of the present disclosure is a MHC class I α chain polypeptide.

The MHC molecule is capable of presenting a peptide of the present disclosure (i.e. in the form of a MHC:peptide complex) to a T cell expressing a TCR that binds to the MHC:peptide complex.

Accordingly. the present disclosure also provides a MHC peptide complex comprising a MHC molecule according to the present disclosure (i.e. comprising a polypeptide of the present disclosure), and a peptide presented by the MHC molecule.

### Cells Comprising/Expressing the Polypeptides and Nucleic Acids/Vectors of the Disclosure

The present disclosure also provides a cell comprising a polypeptide. MHC molecule, MHC:peptide complex, antigen-binding molecule, nucleic acid/plurality or vector/plurality according to the present disclosure.

It will be appreciated that where cells are referred to herein in the singular (i.e. ‘a/the cell’), pluralities/populations of such cells are also contemplated.

The present disclosure further provides a plurality of cells comprising polypeptides according to the present disclosure, wherein the polypeptides are non-identical. In particular, the present disclosure provides a plurality of cells, wherein different cells of the plurality of cells comprise polypeptides comprising non-identical identifier moieties That is. the present disclosure provides a plurality of cells (which may be referred to as a ‘library’) in which different cells of the plurality comprise different polypeptides according to the present disclosure comprising non-identical identifier moieties.

The present disclosure similarly provides a plurality of cells comprising non-identical MHC molecules according to the present disclosure, particularly wherein cells of the plurality comprise MHC molecules comprising polypeptides having non-identical identifier moieties. That is, the present disclosure provides a plurality of cells (a ‘library’) in which different cells of the plurality comprise non-identical MHC molecules according to the present disclosure, wherein the non-identical MHC molecules comprise polypeptides of the present disclosure having non-identical identifier moieties.

The present disclosure similarly provides a plurality of cells comprising non-identical MHC:peptide complexes according to the present disclosure, particularly wherein cells of the plurality comprise MHC:peptide complexes comprise MHC molecules comprising polypeptides having non-identical identifier moieties, and/or wherein cells of the plurality comprise MHC:-peptide complexes comprising non-identical peptides. That is, the present disclosure provides a plurality of cells (a ‘library’) in which different cells of the plurality comprise non-identical MHC peptide complexes according to the present disclosure, wherein the non-identical MHC:peptide complexes comprise MHC: molecules comprising polypeptides having non-identical identifier moieties, and/or wherein the non-identical MHC:peptide complexes comprise non-identical peptides.

The cell may be a eukaryotic cell. e.g. a mammalian cell. The mammal may be a primate (rhesus, cynomolgus, non-human primate or human) or a non-human mammal (e g. rabbit. guinea pig, rat. mouse or other rodent (including any animal in the order Rodentia), cat, dog, pig, sheep. goat, cattle (including cows. e.g dairy cows. or any animal in the order Bos), horse (including any animal in the order Equidae), donkey, and non-human primate). In preferred embodiments, the cell is a human cell.

In some embodiments, the cell is. or is derived from, a cell type commonly used for the expression of polypeptides for use in therapy in humans. Exemplary cells are described e g. in Kunert and Reinhart, Appl Microbiol Biotechnol. (2016) 100.3451-3461 (hereby incorporated by reference in its entirety), and include e.g. CHO. HEK 293. PER C6, NSO and BH K cells. In preferred embodiments, the cell is, or is derived from, a CHO cell.

The present disclosure also provides methods for producing a cell according to the present disclosure, and the cells obtained or obtainable by such methods. Methods for producing cells comprising/expressing α polypeptide] polypeptide complex of interest are well known to the skilled person, and generally comprise introducing nucleic acid(s)/vector(s) encoding the polypeptide(s) of interest into the cells.

Such methods may comprise nucleic acid transfer for permanent (i.e. stable) or transient expression of the transferred nucleic acid. In some embodiments, following introduction into a cell nucleic acid(s) encoding the polypeptide(s) of interest may be integrated into or form part of the genomic DNA of the cell. In some embodiments, following introduction into a cell nucleic acid(s) encoding the polypeptide(s) of interest may be maintained extrachromosomally.

Any suitable genetic engineering platform may be used, and include gammaretroviral vectors. lentiviral vectors, adenovirus vectors. DNA transfection. transposon-based gene delivery and RNA transfection. for example as described in Maus et al., Annu Rev Immunol (2014) 32.189-225, hereby incorporated by reference in its entirety. Methods also include those described e.g. in Wang and Rivire Mol Ther Oncolytics. (2016) 3:16015, which is hereby incorporated by reference in its entirety. Suitable methods for introducing nucleic acid(s)/vector(s) into cells include transduction. transfection and electroporation.

Aspects and embodiments of the present disclosure relate particularly to antigen-presenting cells (APCs). APCs are cells that express MHC molecules (e.g. NIC class I and/or MHC class II molecules), and are capable of presenting MHC:peptide complexes. It will be appreciated that APCs according to the present disclosure are capable of presenting a MHC:peptide complex according to the present disclosure.

APCs according to the present disclosure may be professional APCs. Professional APCs are specialised for presenting antigens to T cells; they are efficient at processing and presenting MIC-peptide complexes at the cell surface, and express high levels of costimulatory molecules.

Professional APCs include dendritic cells (DCs). macrophages, and B cells Non-professional APCs are other cells capable of presenting MHC-peptide complexes to T cells, in particular MHC Class I-peptide complexes to CD8+ T cells.

In some embodiments, the APC is an APC capable of cross-presentation on MHC class I of antigen intemalised by the APC (e.g. taken-up by endocytosis/phagocytosis). Cross-presentation on MHC class I of intemalised antigens to CD8+ T cells is described e.g. in Alloatti et al., Immunological Reviews (2016), 272(1): 97-108. which is hereby incorporated by reference in its entirety. APCs capable of cross-presentation include e g. dendritic cells (DCs), macrophages, B cells and sinusoidal endothelial cells.

In some embodiments, the APC expresses/comprises a peptide presented by a MHC molecule comprising a polypeptide according to the present disclosure.

In some embodiments, the APC expresses/comprises a peptide presented by a MHC molecule according to the present disclosure as a result of having been contacted with the peptide, or having been contacted with α polypeptide comprising the peptide, and having internalised it.

Where the peptide is provided to the APC in the form of α polypeptide comprising the peptide, the polypeptide may be processed by the cell to produce the peptide.

In some embodiments, the APC may have been ‘pulsed’ with the polypeptide/peptide, which may involve culturing APCs in vitro in the presence of the polypeptide/peptide, for a period of time sufficient for the APCs to internalise the polypeptide/peptide.

In some embodiments, the APC expresses/comprises a peptide presented by a MHC molecule according to the present disclosure as a result of expression of nucleic acid encoding the peptide. or expression of nucleic acid encoding a polypeptide comprising the peptide, within the cell. In some embodiments, the APC comprises nucleic acid encoding a peptide presented by a MHC molecule α polypeptide according to the present disclosure, or nucleic acid encoding a polypeptide comprising the peptide. APCs may comprise such nucleic acid as a consequence of having had nucleic acid encoding the peptide/polypeptide (e.g. in the form of a vector comprising such nucleic acid) introduced into the cell, e.g. via transfection. transduction, electroporation, etc.

In some embodiments, the APC is a cell of a cell line (e.g. an immortalised cell line).

In some embodiments, the APC comprises modification to reduce/prevent expression of one or more MHC polypeptides (i e as compared to the level of expression of the relevant MHC polypeptide(s) by an equivalent unmodified cell) In some embodiments, the APC comprises modification to reduce expression of one or more endogenous MHC polypeptides. i.e. MHC polypeptides encoded by the genome of an equivalent unmodified cell. In some embodiments, the APC comprises modification to reduce expression of one or more endogenous MHC polypeptides capable of associating with α polypeptide according to the present disclosure to form a functional MHC molecule (i.e. capable of antigen presentation). In some embodiments, the APC comprises modification to reduce expression of one or more MHC class I α chain polypeptides.

Modification to reduce/prevent expression of one or more MHC polypeptides is contemplated in particular where the APCs are allogeneic with respect to the subject from which the T cells to be screened in accordance with the methods for identifying a TCR according to the present disclosure are isolated/obtained.

Where a subject is referred to herein as being ‘allogeneic’ or ‘non-autologous' with respect to reference subject. the subjects are not the same. The allogeneic subject may be genetically non-identical to the reference subject. The allogeneic subject may comprise MHC/HLA genes encoding MHC/HLA molecules (e.g MHC class I a and/or MHC class 11 molecules) that are non-identical to the MHC/HLA molecules (e.g. MHC class I a and/or MHC class II molecules) encoded by the reference subject.

Conversely. where a subject is referred to herein as being ‘autogeneic’ or ‘autologous’, the subjects may be genetically identical. The autogeneic subject may comprise MHC/HLA genes encoding identical MHC/HLA molecules (e.g. MHC class I (and/or MHC class II molecules). The autogeneic subject may be the same subject.

Modification of a given target nucleic acid can be achieved in a variety of ways known to the skilled person. including modification of the target nucleic acid by homologous recombination, and target nucleic acid editing using site-specific nucleases (SSNs).

Suitable methods may employ targeting by homologous recombination, which is reviewed, for example, in Mortensen Curr Protoc Neurosci. (2007) Chapter 4.Unit 4 29 and Vasquez et al., PNAS 2001. 98(15): 8403-8410 both of which are hereby incorporated by reference in their entirety. Targeting by homologous recombination involves the exchange of nucleic acid sequence through crossover e % ents guided by homologous sequences. Other suitable techniques include nucleic acid editing using SSNs. Gene editing using SSNs is reviewed e.g. in Eid and Mahfouz, Exp Mol Med. 2016 Oct: 48(10): e265. which is hereby incorporated by reference in its entirety. Enzymes capable of creating site-specific double strand breaks (DSBs) can be engineered to introduce DSBs to target nucleic acid sequence(s) of interest. DSBs may be repaired by either error-prone non-homologous end-joining (NHEJ), in which the two ends of the break are rejoined. often with insertion or deletion of nucleotides. Alternatively. DSBs may be repaired by homology-directed repair (HDR), a high-fidelity mechanism in which a DNA template with ends homologous to the break site is supplied and introduced at the site of the DSB.

SSNs capable of being engineered to generate target nucleic acid sequence-specific DSBs include zinc-finger nucleases (ZFNs), transcription activator-like effector nucleases (TALENs) and clustered regularly interspaced palindromic repeats/CRISPR-associated-9 (CRISPR Cas9) systems. ZFN systems are reviewed e g. in Umov et al, Nat Rev Genet. (2010) 11(9):636-46, which is hereby incorporated by reference in its entirety. ZFNs comprise a programmable Zinc Finger DNA-binding domain and a DNA-cleaving domain (e.g. a FokI endonuclease domain).

The DNA-binding domain may be identified by screening a Zinc Finger array capable of binding to the target nucleic acid sequence. TALEN systems are reviewed e.g. in Mahfouz et al. Plant Biotechnol J. (2014) 12(8):1006-14, which is hereby incorporated by reference in its entirety. TALENs comprise a programmable DNA-binding TALE domain and a DNA-cleaving domain (e.g. a FokI endonuclease domain). TALEs comprise repeat domains consisting of repeats of 33-39 amino acids, which are identical except for two residues at positions 12 and 13 of each repeat which are repeat variable di-residues (RVDs). Each RVD determines binding of the repeat to a nucleotide in the target DNA sequence according to the following relationship: ‘HD’ binds to C. ‘NI’ binds to A. ‘NG’ binds to T and ‘NN’ or ‘NK’ binds to G (Moscou and Bogdanove. Science (2009) 326(5959):1501.) CR.ISPR/Cas9 and related systems e.g. CRISPR/Cpf1, CR.ISPR/C2cl, CRISPR/C2c2 and CRISPR % C2c3 are reviewed e.g. in Nakade et al., Bioengineered (2017) 8(3):265-273, which is hereby incorporated by reference in its entirety. These systems comprise an endonuclease (e.g. Cas9. Cpf1 etc.) and the single-guide RNA (sgRNA) molecule. The sgRNA can be engineered to target endonuclease activity to nucleic acid sequences of interest.

In some embodiments - and particularly embodiments in which the APC comprises modification to reduce/prevent expression of one or more endogenous MHC polypeptides - the APC further comprises modification to comprise/express nucleic acid encoding one or more MHC polypeptides. In such embodiments, the APC is preferably to engineered to express M1C polypeptide(s) corresponding to those that the APC comprises modification to reduce/prevent endogenous expression of (for example, if the APC comprises modification to reduce/prevent expression of an endogenous MHC class I α chain polypeptide, then the APC is engineered to express the relevant MHC class I α chain polypeptide). The APC may be engineered to express one or more MHC polypeptides encoded by the genome of a subject of interest. e.g. a patient.

The subject of interest may be the same subject from which the T cells to be screened in accordance with the methods for identifying a TCR according to the present disclosure are isolated/obtained. In some embodiments, the APC is engineered to express one or more MHC polypeptides encoded by a subject comprising a disease-associated antigen according to the present disclosure. In some embodiments, the APC is engineered to express one or more MHC polypeptides encoded by a subject comprising a cancer-associated neoantigen (i e a subject comprising cells comprising/expressing the cancer-associated neoantigen).

The preceding paragraph relates particularly to embodiments in which the articles and methods of the present disclosure are employed for the identification of TCRs binding to MHC:complexes of particular interest. Modification of the APC to reduce, prevent expression of endogenous MHC polypeptide(s), and engineering of the APC to express MHC polypeptide(s) encoded by the genome of the subject of interest. provides for antigen presentation as it would occur in the subject.

In some embodiments, the APC is a primary cell, e.g. a cell isolated/obtained directly from a live subject/tissue (e g. via biopsy). The APC may have been isolated/obtained from or derived from cells isolated obtained from a subject of interest, e.g. a patient (e.g a subject/patient comprising a disease-associated antigen according to the present disclosure, e.g. a subject/patient comprising a cancer-associated neoantigen (i.e. a subject/patient comprising cells comprising/expressing the cancer-associated neoantigen). In such embodiments, the APC may be from an autogeneic subject to the subject from which the ‘T cells to be screened in accordance with the methods for identifying a TCR according to the present disclosure are isolated/obtained. In such embodiments, it is not generally contemplated that the APC is modified to reduce/prevent expression of an endogenous MHC polypeptide, as the APC already expresses appropriate MHC molecules for presentation of the peptides of interest as the-v occur in the subject of interest.

The present disclosure provides a method for producing a cell comprising a MHC molecule labelled with an identifier moiety. comprising: (1) introducing into a cell (e.g an APC) a nucleic acid or plurality of nucleic acids encoding a polypeptide comprising (i) the amino acid sequence of a MHC polypeptide (e.g. β2 microglobulin), and (ii) a moiety facilitating labelling of the polypeptide with an identifier moiety (e.g a HaloTag®); and (2) contacting the cell with a labelling moiety comprising an identifier moiety, wherein the labelling moiety is suitable for labelling the polypeptide encoded by the nucleic acid or plurality of nucleic acids of (1) with an identifier moiety (e.g. contacting the cell with a HaloTag® ligand comprising a ssDNA moiety and a chloroalkane moiety).

The present disclosure also provides a method for producing a cell comprising a MHC:peptide complex comprising a MHC molecule labelled with an identifier moiety. comprising. (1) introducing into a cell (e.g an APC) a nucleic acid or plurality of nucleic acids encoding a polypeptide comprising (i) the amino acid sequence of a MHC polypeptide (e.g. β2 microglobulin), and (ii) a moiety facilitating labelling of the polypeptide with an identifier moiety (e g, a Halo‘ag’); (2) introducing into the cell: (i) a peptide presented by a MHC molecule comprising a polypeptide encoded by the nucleic acid or plurality of nucleic acids of (1). or (ii) nucleic acid encoding a peptide presented by a MHC molecule comprising a polypeptide encoded by the nucleic acid or plurality of nucleic acids of (1), and (3) contacting the cell with a labelling moiety comprising an identifier moiety, wherein the labelling moiety is suitable for labelling the polypeptide encoded by the nucleic acid or plurality of nucleic acids of (1) with an identifier moiety (e.g contacting the cell with a HaloTag® ligand comprising a ssDNA moiety and a chloroalkane moiety).

In accordance with the preceding two paragraphs, in some embodiments the cell is an APC. e.g an APC as described herein. In some embodiments, the methods further comprise method steps for producing an APC as described herein. e.g. by modification to reduce/prevent expression of one or more endogenous MHC polypeptides (e.g. in the case of APCs allogeneic to the subject from which the ‘T cells to be screened in accordance with the methods of the disclosure are isolated/obtained), and/or modification to express one or more non-endogenous MHC polypeptides (e.g. one or more MHC polypeptides encoded by the genome of the subject from which the T cells to be screened in accordance with the methods of the disclosure are isolated/obtained).

### Compositions

The present disclosure provides compositions comprising the polypeptides, antigen-binding molecules, nucleic acids. expression vectors and cells of the present disclosure.

The present disclosure also provides compositions comprising a cell (e.g. an antigen-presenting cell) according to the present disclosure, and a T cell.

The present disclosure also provides a composition comprising:
(α) a cell (e.g. an antigen-presenting cell) comprising a polypeptide according to the present disclosure (e g, α polypeptide comprising a moiety facilitating labelling of the polypeptide with an identifier moiety, or α polypeptide comprising an identifier moiety) at its cell surface, and
(b) a cell (e.g. a T cell) comprising a polypeptide comprising a sortase acceptor motif at its cell surface; and
(c) a sortase.

In some embodiments, the composition comprises.
(α) a cell (e.g an antigen-presenting cell) comprising a polypeptide according to the present disclosure (e.g. polypeptide comprising a moiety facilitating labelling of the polypeptide with an identifier moiety, or α polypeptide comprising an identifier moiety) at its cell surface. and
(b) a cell (e.g a T cell) comprising at its cell surface. (i) α polypeptide comprising a sortase acceptor motif, and (ii) a sortase.

It will be appreciated that cells according to the present disclosure are contemplated to be employed in methods in which T cells according to the present disclosure are contacted with cells (e g APCs) according to the present disclosure. for the purposes of identifying T cells expressing TCRs that bind to MHC:peptide complexes presented by the cells.

It will of course be appreciated that a composition according to the present disclosure may comprise a plurality of cells according to the present disclosure, and a plurality of T cells.

In some embodiments, the composition comprises a plurality of non-identical T cells. In some embodiments, the composition comprises a plurality of T cells encoding/comprising non-identical TCRs. It will be appreciated that in embodiments of the methods of the present disclosure, antigen-presenting cells according to the present disclosure are contacted with a population of T cells encoding/comprising a diversity of TCRs for the ultimate identification of TCRs capable of binding to MHC:peptide complexes presented by the antigen-presenting cells.

In some embodiments, the composition according to the present disclosure comprises a plurality of non-identical cells according to the present disclosure. In some embodiments, the composition comprises a plurality of antigen-presenting cells comprising/presenting non-identical MHC:peptide complexes. It will be appreciated that in embodiments of the methods of the 20 present disclosure, a plurality of antigen-presenting cells according to the present disclosure are engineered to express/comprise non-identical peptides for presentation by MHC peptide complexes, and contacted with a population of T cells encoding/comprising a diversity of TCRs for the ultimate identification of TCRs capable of binding to the various different MHC:peptide complexes presented by the antigen-presenting cells.

### Methods for Identifying a T Cell Receptor-Binding MHC:Peotide Complexes, and Methods

for Identifying Cells that have Interacted with One Another Aspects and embodiments of the present disclosure are concerned in particular with the identification of T cell receptors (TCRs) that bind to MHC:peptide complexes. In particular, it is contemplated to employ the articles described herein (e.g the polypeptides. MHC molecules, MHC:peptide complexes, antigen-binding molecules, and cells according to the present disclosure) in methods for the identification of TCRs that bind to MHC peptide complexes comprising peptides of interest.

Accordingly. the present disclosure provides a method for identifying a T cell receptor (TCR) that binds to a MHC:peptide complex, comprising: (1) contacting (α) a cell comprising a MHC:peptide complex with (b) a population of T cells. (2) incubating the cells obtained after step (1) under conditions suitable for trogocytosis of a MHC:peptide complex by a T cell comprising a TCR that binds to the MHC:peptide complex; and (3) subsequently analysing the cells obtained after step (2) to identify a TCR that binds to the MHC:peptide complex: wherein the MHC:peptide complex comprises a MHC molecule, and wherein the MHC molecule comprises a polypeptide comprising (i) the amino acid sequence of a MHC polypeptide, and (ii) an identifier moiety. wherein the identifier moiety is covalently associated with the polypeptide via linkage formed by a self-labelling protein tag.

The present disclosure also provides a method for identifying a T cell receptor (TCR) that binds to a MHC:peptide complex. comprising: (1) contacting (a) a cell comprising a MHC peptide complex with (b) a population of cells comprising T cells comprising a polypeptide comprising a sortase acceptor motif at the cell surface, in the presence of a sortase: (2) incubating the cells obtained after step (1) under conditions suitable for interaction between the cell of (a) and the population of cells of (b); and (3) subsequently analysing the cells obtained after step (2) to identify a T cell comprising a TCR that binds to the MHC:peptide complex: wherein the MHC:peptide complex comprises a MHC molecule, and wherein the MHC molecule comprises a polypeptide comprising (i) the amino acid sequence of a MHC polypeptide, (ii) a sortase substrate motif, and (iii) an identifier moiety, wherein the identifier moiety is covalently associated with the polypeptide via linkage formed by a self-labelling protein tag. In some embodiments, incubating the cells (i. the co-culture of cells) under conditions suitable for interaction between the cell of (α) and the population of cells of (b) comprises culture under conditions suitable for sortase-mediated transfer of the identifier moiety (i.e. from the polypeptide comprising (i) the amino acid sequence of a MHC polypeptide, (ii) a sortase substrate motif, and (iii) an identifier moiety) to the polypeptide comprising a sortase acceptor motif.

The cell comprising a MHC:peptide complex comprising a MHC molecule comprising a polypeptide according to the present disclosure may be a cell as described herein. e.g an APC. The step of ‘contacting’ such cells with T cells may comprise bringing the cells into contact with one another, in a co-culture.

Incubating the cells (i.e. the co-culture of cells) under conditions suitable for trogocytosis of a MHC:peptide complex by a T cell comprising a TCR that binds to the MHC:peptide complex may comprise maintaining the cells at 37° C. in a humidified atmosphere containing 5% CO₂. Similarly, incubating the cells (i.e. the co-culture of cells) under conditions suitable for interaction between the cell of (a) and the population of cells of (b) may comprise maintaining the cells at 37° C. in a humidified atmosphere containing 5% CO₂. Similarly, incubating the cells (i.e. the co-culture of cells) under conditions suitable for interaction between the cell of (α) and the population of cells of (b) comprises culture under conditions suitable for sortase-mediated transfer of the identifier moiety to the polypeptide comprising a sortase acceptor motif may comprise maintaining the cells at 37° C. in a humidified atmosphere containing 5% CO₂. Appropriate culture conditions can readily be determined by the skilled person.

Trogocytosis refers to intemalisation by one cell of cell membrane and/or cell-membrane-bound material of another cell. Trogocytosis is described e.g. in Zhao et al., Front Immunol. (2022) 13: 791006, which is hereby incorporated by reference in its entirety. The present disclosure is particularly interested in trogocytosis by T cells of MHC:peptide complexes presented by APCs.

In some embodiments, a sortase is provided at the cell surface of T cells comprising a polypeptide comprising a sortase acceptor motif The sortase may be immobilised on the T cell, i.e. on or at the cell surface of the T cell. In some embodiments, the sortase is covalently associated with a molecule in or at the cell membrane of the T cell. In some embodiments, the sortase is non-covalently associated (e.g through protein:protein interaction, e.g through antibody:antigen interaction) with a molecule in or at the cell membrane of the T cell.

In some aspects and embodiments, the methods of the present disclosure further comprise analysing the T cells in order to identify TCRs that bind to MHC:peptide complexes.

In some embodiments, the methods further comprise determining the peptide of a MHC:peptide complex internalised by the T cell. In preferred embodiments, analysing the T cells comprises analysis of a T cell to determine the identity of the identifier moiety (i.e. the identifier moiety of the MHC:peptide complex, or the identifier moiety transferred to the polypeptide comprising a sortase acceptor motif). As explained hereinabove, a given identifier moiety is preferably employed with and codes for a particular peptide, and therefore determination of the identity of the identifier moiety provides for determination of the peptide of the MHC:peptide complex with which the T cell has interacted (and which may e.g. have been intemalised by the T cell) The analysis employed to determine the identity of the identifier moiety is of course selected in accordance with the nature of the identifier moiety. In embodiments wherein the identifier moiety comprises or consists of a nucleic acid motets. the analysis may comprise determining the structure of the nucleic acid moiety. In some embodiments, the analysis comprises determining the nucleotide sequence of an identifier moiety comprising or consisting of pol nucleotide.

The nucleotide sequence of a polynucleotide/nucleic acid may be determined by any suitable techniques, which are % well known to the skilled person, and include e.g sanger sequencing. In preferred embodiments, next-generation sequencing (NGS) technology may be employed.

In preferred embodiments, analysing the T cells further comprises analysis of a T cell to determine the identity of the TCR encoded by the T cell. Determining the identity of the TCR encoded by a T cell mas comprise determining the nucleotide sequence of nucleic acid encoding the variable regions of the TCR, and/or determining the amino acid sequence of the variable regions of the ‘CR (e.g. via in silico translation of the encoding nucleic acid sequence) In some embodiments, the methods comprise determining the nucleotide sequence of gene regions encoding the variable region of a TCRα chain (e.g. the Va and/or Ja regions), and/or determining the nucleotide sequence of gene regions encoding the variable region of a TCRβ chain (e.g. the Vβ, Dβ and/or Jβ regions).

It will be appreciated that in some embodiments, the methods comprise determining the peptide of a MHC:peptide complex intemalised by a T cell (e.g. via determination of the identity of the identifier moiety ) and determining the identity of the TCR encoded by the T cell. It will similarly be appreciated that in some embodiments, the methods comprise determining the peptide of a MHC:peptide complex that has interacted with a T cell (e.g. via determination of the identity of the identifier moiety transferred to the T cell) and determining the identity of the TCR encoded by the T cell. In this way, the methods provide for the facile identification of particular TCRs that recognise MHC:peptide complexes presenting peptides of particular interest. In preferred embodiments, the identifier moiety comprises or consists of a nucleic acid moiety (e.g. a polynucleotide), and the methods comprise analysis of the identifier moiety and the nucleotide sequence of nucleic acid encoding the variable regions of the TCR using next-generation sequencing (NGS) technology.

It will be appreciated that the analysis is performed on a single cell having intemalised a MHC:peptide complex according to the present disclosure, or is performed on a single cell having had an identifier moiety transferred thereto via sortase-mediated transfer. Single-cell sequencing methods are reviewed e.g. in Tang et al, Cell & Bioscience (2019) 9. 53, which is hereby incorporated by reference in its entirety

The use of a plurality of identifier moieties and corresponding peptides and a population of a diversity of T cells encoding different TCRs provides for the simultaneous identification of multiple TCR-MHC:peptide complex interaction pairs. in a single experiment

In some embodiments of the methods of the present disclosure, APCs comprising MHC molecules comprising a polypeptide according to the present disclosure comprising (i) the amino acid sequence of a MHC polypeptide, and (ii) a moiety facilitating labelling of the polypeptide with an identifier moiety may be modified to comprise peptides according to the present disclosure (e.g. via pulsing the APCs with the peptides comprising relevant peptides, or via introduction into the APC of nucleic acid encoding the peptides), and the APCs may also labelled with identifier moieties corresponding to the peptides (e.g. using a labelling moiety) APCs may be labelled with different peptide and identifier pairs e.g. in wells of a polypropylene plate. The various peptide- and identifier-labelled APCs may then be pooled, and contacted with a population of T cells, and incubated under conditions suitable for trogocytosis of a MHC:peptide complexes by T cells comprising a TCR that binds to a MHC:peptide complex. T cells of the population may then be analysed to determine the identity of the TCR encoded by the T cell, and the identity of the peptide of the MHC:peptide complex internalised by the T cell. i.e. via determination of the identity of the identifier moiety.

In some embodiments of the methods of the present disclosure, APCs comprising MHC molecules comprising a polypeptide according to the present disclosure comprising (i) the amino acid sequence of a MHC polypeptide. (ii) a sortase substrate motif, and (iii) a moiety facilitating labelling of the polypeptide with an identifier moiety may be modified to comprise peptides according to the present disclosure (e.g. via pulsing the APCs with the peptides comprising relevant peptides. or via introduction into the APC of nucleic acid encoding the peptides), and the APCs may also be labelled with identifier moieties corresponding to the peptides (e g. using a labelling moiety). APCs may be labelled with different peptide and identifier pairs e g. in wells of a polypropylene plate. The various peptide- and identifier-labelled APCs may then be pooled. and contacted with a population of T cells comprising a polypeptide comprising a sortase acceptor motif at the cell surface. in the presence of a sortase, and incubated under conditions suitable for sortase-mediated transfer of the identifier moieties to the polypeptide comprising a sortase acceptor motif. T cells of the population may then be analysed to determine the identity of the TCR encoded by the T cell, and the identity of the peptide of the MHC:peptide complex that has interacted with the T cell (e.g. via determination of the identity of the identifier moiety transferred to the T cell)

It should be appreciated that in some embodiments it 6ill be possible that a T cell expressing a given single TCR may recognise, and therefore may interact with and/or intemalise. multiple. non-identical peptide:MHC complexes. In such embodiments. multiple different identifier moieties may be detected in or on a given T cell.

In some embodiments, the methods may further comprise a step of isolating/selecting T cells having participated in, or being likely to have participated in. formation of a TCR-MHC:peptide complex (i e prior to analysis of the T cells to determine the identity of the T cell and identifier moiety). In accordance with such embodiments, the cells are selected/isolated (i.e. from other cells. e.g. T cells that have not/are unlikely to have participated in the formation of such a complex) for subsequent analysis. This method step focusses downstream analysis on those T cells having interacted with and/or intemalised MHC:peptide complexes of interest.

In some embodiments, the T cells are isolated/selected on the basis of characterisation of a correlate of CD3-TCR complex-mediated signalling, e.g. a marker of proliferation/population expansion, growth factor (e.g. IL-2) expression, IFNγ expression, CD107a expression, TNFα expression, GM-CSF expression, perforin expression, granzyme expression, granulysin expression, and/or FAS ligand (FASL) expression

In some embodiments, particularly embodiments wherein the polypeptide of the present disclosure comprises a detectable moiety as described herein. T cells may be isolated/selected for subsequent analysis on the basis of detection of the detectable marker in or on the T cell. By way of illustration, in an embodiment wherein the polypeptide of the present disclosure comprises a fluorescent label. T cells having internalised MHC:peptide complexes comprising the polypeptide of the present disclosure may be detected and sorted (e.g. by fluorescence activated cell sorting (FACS)), based on detection of T cells comprising the fluorescent label. The T cells comprising the fluorescent label may then by further analysed as described above to determine the identity of the TCR and the identity of the peptide of the peptide:MHC complexes they have internalised.

It will be appreciated that in some embodiments, the methods for identifying a T cell receptor-binding MHC:peptide complexes additionally comprise method steps for the production of APCs comprising MHC molecules according to the present disclosure, and/or for the production of APCs comprising MHC:peptide complexes according to the present disclosure, as described hereinabove.

In further aspects and embodiments, the present disclosure more generally provides methods for identifying a cell that has interacted with a cell comprising a polypeptide comprising (i) a sortase substrate motif, and (ii) an identifier moiety at its cell surface, comprising. (1) contacting (α) a cell comprising a polypeptide comprising (i a sortase substrate motif, and (ii) an identifier moiety at its cell surface with (b) a population of cells comprising a cell comprising a polypeptide comprising a sortase acceptor motif at its cell surface, in the presence of a sortase; and (2) incubating the population of cells obtained after step (1) under conditions suitable for interaction between the cell of (α) and the population of cells of (b); and (3) subsequently analysing the cells obtained after step (2) to identify a cell within the population of cells of (b) that has undergone sortase-mediated transfer of an identifier moiety, thereby identifying a cell that has interacted with a cell comprising (i) a sortase substrate motif, and (ii) an identifier moiety. In some embodiments, incubating the cells (i.e. the co-culture of cells) under conditions suitable for interaction between the cell of (a) and the population of cells of (b) comprises culture tinder conditions suitable for sortase-mediated transfer of the identifier moiety (i.e. from the polypeptide comprising (i) a sortase substrate motif, and (ii) an identifier moiety) to the polypeptide comprising a sortase acceptor motif.

The step of ‘contacting’ cells of (a) with cells of (b) may comprise bringing the cells into contact with one another, in a co-culture.

Incubating the cells (i.e. the co-culture of cells) under conditions suitable for interaction between the cell of (a) and the population of cells of (b) comprises culture under conditions suitable for sortase-mediated transfer of the identifier moiety to the polypeptide comprising a sortase acceptor motif, which may comprise maintaining the cells at 37° C. in a humidified atmosphere containing 5% CO₂ Appropriate culture conditions can readily be determined by the skilled person.

In some embodiments, a sortase is provided at the cell surface of cells comprising a polypeptide comprising a sortase acceptor motif. The sortase may be immobilised on the cell. i.e. on or at the cell surface of the cell. In some embodiments, the sortase is covalently associated with a molecule in or at the cell membrane of the T cell. In some embodiments, the sortase is non-covalently associated (e.g. through protein:protein interaction, e.g. through antibody:antigen interaction) with a molecule in or at the cell membrane of the cell.

The analysis employed to identify a cell within the population of cells of (b) that has undergone sortase-mediated transfer of an identifier moiety is of course selected in accordance with the nature of the identifier moiety. In embodiments wherein the identifier moiety comprises or consists of a nucleic acid moiety, the analysis may comprise determining the structure of the nucleic acid moiety. In some embodiments, the analysis comprises determining the nucleotide sequence of an identifier moiety comprising or consisting of polynucleotide. The nucleotide sequence of a polynucleotide/nucleic acid may be determined by any suitable techniques, as described hereinabove.

It will be appreciated that the analysis is performed on a single cell having had an identifier moiety transferred thereto via sortase-mediated transfer. Single-cell sequencing methods are reviewed e.g in Tang et al., Cell & Bioscience (2019) 9: 53, which is hereby incorporated by reference in its entirety The use of a plurality of identifier moieties and a population of a diversity of cells provides for the simultaneous identification of multiple cell-cell interaction pairs, in a single experiment.

In some embodiments. cells comprising a polypeptide comprising (i) a sortase substrate motif. and (ii) a moiety facilitating labelling of the polypeptide with an identifier moiety may be labelled with different identifier moieties e.g in wells of a polypropylene plate. The cells may then be pooled, and contacted with a population of cells comprising a polypeptide comprising a sortase acceptor motif at the cell surface, in the presence of a sortase, and incubated under conditions suitable for sortase-mediated transfer of the identifier moieties to the polypeptide comprising a sortase acceptor motif

In some embodiments, the methods may further comprise a step of isolating/selecting cells having participated in, or being likely to have participated in, cell-cell interaction (i.e. prior to analysis of the cells to determine the identity of the identifier moiety). In accordance with such embodiments, the cells are selected/isolated (i.e. from other cells, e.g. cells that have not/are unlikely to have participated in such interaction) for subsequent analysis. This method step focusses downstream analysis on those cells having participated in cell-cell interactions of interest.

In some embodiments, particularly embodiments wherein the polypeptide of the present disclosure comprises a detectable moiety as described herein, cells may be isolated/selected for subsequent analysis on the basis of detection of the detectable marker in or on the cell.

It will be appreciated that in some embodiments, the methods additionally comprise method steps for producing cells comprising a polypeptide comprising (1) a sortase substrate motif, and (ii) an identifier moiety at their cell surface, as described hereinabove.

### Kits

The present disclosure also provides kits of parts. Aspects and embodiments of the present disclosure relate to kits for producing a cell (e.g. an antigen-presenting cell) according to the present disclosure. Aspects and embodiments of the present disclosure relate to kits for performing the methods according to the present disclosure.

In some aspects, a kit according to the present disclosure comprises. (1) a nucleic acid or plurality of nucleic acids according encoding a polypeptide according to the present disclosure; and (2) a labelling moiety comprising an identifier moiety. wherein the labelling moiety is suitable for labelling the polypeptide encoded by the nucleic acid or plurality of nucleic acids according to (1) with the identifier moiety via the moiety facilitating labelling of the polypeptide with an identifier moiety.

In some aspects, a kit according to the present disclosure comprises: (1) a nucleic acid, or a plurality of nucleic acids, encoding a polypeptide comprising (i) the amino acid sequence of β2 microglobulin, and (ii) a HaloTag®, and (2) a HaloTag. ligand comprising a ssDNA moiety and a chloroalkane moiety.

In some aspects, a kit according to the present disclosure comprises (1) a nucleic acid, or a plurality of nucleic acids, encoding a polypeptide comprising (i) the amino acid sequence of β2 microglobulin, (ii) a sortase substrate motif, and (iii) a HaloTag®; and (2) a HaloTag® ligand comprising a ssDNA moiety and a chloroalkane moiety. In some embodiments, the kit further comprises a sortase, and reagents for modifying cells to comprise a polypeptide comprising a sortase acceptor motif at the cell surface. In some embodiments, the kit comprises reagents for modifying cells to comprise a sortase at the cell surface.

In some embodiments, the kit further comprises: (3) a peptide presented by a MHC molecule comprising a polypeptide encoded by the nucleic acid or plurality of nucleic acids according to (1), or nucleic acid encoding a peptide presented by a MHC molecule comprising a polypeptide encoded by the nucleic acid or plurality of nucleic acids according to (1).

In some aspects, a kit according to the present disclosure comprises (1) a cell comprising a MHC molecule according to the present disclosure, or a plurality of such cells, and (2) a peptide presented by a MHC molecule of a cell according to (1), or nucleic acid encoding a peptide presented by a MHC molecule of a cell according to (1) In some aspects, a kit according to the present disclosure comprises a cell comprising a MHC:peptide complex. comprising a MHC molecule comprising a polypeptide according to the present disclosure, and a peptide presented by the MHC molecule, or a plurality of such cells.

In some embodiments, cells of the plurality of cells in accordance with the preceding two paragraphs are non-identical. In some embodiments, a plurality of cells of a kit according to the present disclosure comprises cells comprising MHC molecules, wherein the MHC molecules expressed by different cells of the plurality comprise polypeptides of the present disclosure labelled with non-identical identifier moieties. In some embodiments, a plurality of cells of a kit according to the present disclosure comprises cells comprising non-identical peptides. Kits of parts according to the present disclosure may comprise a predetermined quantity of the articles described in the preceding six paragraphs. In some embodiments, the relevant articles are provided in containers (e.g in vials or bottles). The kit may provide the relevant articles together with instructions (e g, a protocol) as to how to employ them in accordance with a method described herein.

In some embodiments, a kit of parts comprises materials for producing α polypeptide according to the present disclosure In some embodiments, a kit of parts comprises materials for producing a MHC molecule according to the present disclosure. In some embodiments, a kit of parts comprises materials for producing a MHC:peptide complex according to the present disclosure. In some embodiments, a kit of parts comprises materials for producing a cell according to the present disclosure. In some embodiments, a kit of parts comprises materials for producing a composition according to the present disclosure.

In some embodiments, the kit of parts may comprise a nucleic acid/plurality or an expression vector/plurality according to the present disclosure, and optionally materials for introducing the nucleic acid/plurality or an expression vector/plurality into a cell.

In some embodiments. reagents for modifying cells to comprise a polypeptide comprising a sortase acceptor motif at the cell surface comprise a nucleic acid/plurality encoding a polypeptide comprising a sortase acceptor motif, or an expression vector/plurality comprising such nucleic acid, and optionally materials for introducing the nucleic acid/plurality or an expression vector/plurality into a cell. However, in preferred embodiments, the reagents for modifying cells to comprise a polypeptide comprising a sortase acceptor motif at the cell surface are reagents providing for non-genetic bioconjugation of the polypeptide to the cell. In some embodiments, the reagents comprise ManNAz. In some embodiments, the reagents comprise reagents for functionalising α polypeptide comprising a sortase acceptor motif with a cycloalkyne moiety (e.g a dibenzocyclooctyl (DBCO) moiety or an azadibenzocyclooctyne (ADIBO) moiety).

In some embodiments. reagents for modifying cells to comprise a sortase at the cell surface comprise a nucleic acid/plurality encoding a polypeptide comprising a sortase acceptor motif. or an expression vector/plurality comprising such nucleic acid, and optionally materials for introducing the nucleic acid/plurality or an expression vector/plurality into a cell. In some embodiments, the reagents for modifying cells to comprise a sortase at the cell surface are reagents providing for non-genetic bioconjugation of the polypeptide to the cell. In some embodiments, the reagents comprise ManNAz. In some embodiments, the reagents comprise reagents providing for non-genetic bioconjugation of a hapten (e.g. DOTAM or DOTA) to the cell. In some embodiments, the reagents comprise a compound comprising a cycloalkyne moiety (e.g. a DBCO moiety or an ADIBO moiety) and a hapten (e g. DOTAM or DOTA).

In some embodiments, the reagents comprise an antigen-binding molecule according to the present disclosure, i e an antigen-binding molecule that binds to a hapten (e.g. DOTAM or DOTA), comprising a sortase moiety.

The manufacture of kits of parts according to the present disclosure preferably follows standard procedures which are known to the person skied in the art.

### Sequence Identity

As used herein. ‘sequence identity’ refers to the percent of nucleotides./amino acid residues in a subject sequence that are identical to nucleotides, amino acid residues in a reference sequence, after aligning the sequences and, if necessary, introducing gaps. to achieve the maximum percent sequence identity between the sequences. Pairwise and multiple sequence alignment for the purposes of determining percent sequence identity between two or more amino acid or nucleic acid sequences can be achieved in % various ways known to a person of skill in the art. for instance, using publicly available computer software such as Clustal Omega (Soding, J. 2005, Bioinformatics 21., 951-960). T-coffee (Notredame Et al. 2000, J. Mol. Biol. (2000) 302. 205-217). Kalign (Lassmann and Sonnhamnmer 2005. BMC Bioinformatics, 6(298)) and MAFFT (Katoh and Standley 2013. Molecular Biologr and Evolution. 30(4) 772-780) software. When using such software, the default parameters, e.g. for gap penalty and extension penalty, are preferably used.

### Sequences

| SEQ | | |
|---|---|---|
| ID | | |
| NO: | DESCRIPTION | SEQUENCE |
| 1 | Human β2 | MSRSVALAVLALLSLSGLEAIQRTPKIQVYSRHPAENGKSNFLNCYVS |
| | microglobulin | GFHPSDIEVDLLKNGERIEKVEHSDLSFSKDWSFYLLYYTEFTPTEKDE |
| | (UniProt: P61769) | YACRVNHVTLSQPKIVKWDRDM |
| 2 | Human β2 | MSRSVALAVLALLSLSGLEA |
| | microglobulin signal | |
| | peptide | |
| 3 | Mature human β2 | IQRTPKIQVYSRHPAENGKSNFLNCYVSGFHPSDIEVDLLKNGERIEKV |
| | microglobulin | EHSDLSFSKDWSFYLLYYTEFTPTEKDEYACRVNHVTLSQPKIVKWDR |
| | (lacking signal | DM |
| | peptide) | |
| 4 | HaloTag ® | MAEIGTGFPFDPHYVEVLGERMHYVDVGPRDGTPVLFLHGNPTSSYV |
| | | WRNIIPHVAPTHRCIAPDLIGMGKSDKPDLGYFFDDHVRFMDAFIEAL |
| | | GLEEVVLVIHDWGSALGFHWAKRNPERVKGIAFMEFIRPIPTWDEWPE |
| | | FARETFQAFRTTDVGRKLIIDQNVFIEGTLPMGVVRPLTEVEMDHYRE |
| | | PFLNPVDREPLWRFPNELPIAGEPANIVALVEEYMDWLHQSPVPKLLF |
| | | WGTPGVLIPPAEAARLAKSLPNCKAVDIGPGLNLLQEDNPDLIGSEIAR |
| | | WLSTLEISG |
| 5 | HaloTag ® | AEIGTGFPFDPHYVEVLGERMHYVDVGPRDGTPVLFLHGNPTSSYVW |
| | (lacking N-terminal | RNIIPHVAPTHRCIAPDLIGMGKSDKPDLGYFFDDHVRFMDAFIEALGL |
| | ‘M’ residue) | EEVVLVIHDWGSALGFHWAKRNPERVKGIAFMEFIRPIPTWDEWPEFA |
| | | RETFQAFRTTDVGRKLIIDQNVFIEGTLPMGVVRPLTEVEMDHYREPFL |
| | | NPVDREPLWRFPNELPIAGEPANIVALVEEYMDWLHQSPVPKLLFWG |
| | | TPGVLIPPAEAARLAKSLPNCKAVDIGPGLNLLQEDNPDLIGSEIARWL |
| | | STLEISG |
| 6 | G4S | GGGGS |
| 7 | (G4S)3 linker | GGGGGGGGSGGGGS |
| 8 | (G4S)4 linker | GGGGSGGGGSGGGGGGGGS |
| 9 | Mature HLA-A02 | GSHSMRYFFTSVSRPGRGEPRFIAVGYVDDTQFVRFDSDAASQRMEPR |
| | (positions 22 to 362 | APWIEQEGPEYWDGETRKVKAHSQTHRVDLGTLRGYYNQSEAGSHT |
| | of UniProt: O78126) | VQRMYGCDVGSDWRFLRGYHQYAYDGKDYIALKEDLRSWTAADMA |
| | | AQTTKHKWEAAHVAEQLRAYLEGTCVEWLRRYLENGKETLQRTDAP |
| | | KTHMTHHAVSDHEATLRCWALSFYPAEITLTWRDGEDQTQDTELVE |
| | | TRPAGDGTFQKWAAVVVPSGQEQRYTCHVQHEGLPKPLTLRWEPSSQ |
| | | PTIPIVGIIAGLVLFGAVITGAVVAAVMWRRKSSDRKGGSYSQAASSDS |
| | | AQGSDVSLTACKV |
| 10 | HaloTag ®-B2M | MSRSVALAVLALLSLSGLEAAFIGTGFPFDPHYVEVLGERMHYVDVG |
| | fusion | PRDGTPVLFLHGNPTSSYVWRNIIPHVAPTHRCIAPDLIGMGKSDKPDL |
| | [SignalP]- | GYFFDDHVRFMDAFIEALGLEEVVLVIHDWGSALGFHWAKRNPERVK |
| | [HaloTag ®]-[B2M] | GIAFMEFIRPIPTWDEWPEFARETFQAFRTTDVGRKLIIDNVFIEGTLP |
| | | MGVVRPLTEVEMDHYREPFLNPVDREPLWRFPNELPIAGEPANIVALV |
| | | EEYMDWLHQSPVPKLLFWGTPGVLIPPAEAARLAKSLPNCKAVDIGP |
| | | GLNLLQEDNPDLIGSEIARWLSTLEISGGGGGSGGGGSGGGGSIQRTPK |
| | | IQVYSRHPAENGKSNFLNCYVSGFHPSDIEVDLLKNGERIEKVEHSDLS |
| | | FSKDWSFYLLYYTEFTPTEKDEYACRVNHVTLSQPKIVKWDRDM |
| 11 | Mature HaloTag ® | AEIGTGFPFDPHYVEVLGERMHYVDVGPRDGTPVLFLHGNPTSSYVW |
| | B2M fusion (lacking | RNIIPHVAPTHRCIAPDLIGMGKSDKPDLGYFFDDHVRFMDAFIEALGL |
| | signal peptide) | EEVVLVIHDWGSALGFHWAKRNPERVKGIAFMEFIRPIPTWDEWPEFA |
| | [HaloTag ®]-[B2M] | RETFQAFRTTDVGRKLIIDQNVFIEGTLPMGVVRPLTEVEMDHYREPFL |
| | | NPVDREPLWRFPNELPIAGEPANIVALVEEYMDWLHQSPVPKLLFWG |
| | | TPGVLIPPAEAARLAKSLPNCKAVDIGPGLNLLQEDNPDLIGSEIARWL |
| | | STLEISGGGGGSGGGGSGGGGSIQRTPKIQVYSRHPAENGKSNFLNCY |
| | | VSGFHPSDIEVDLLKNGERIEKVEHSDLSFSKDWSFYLLYYTEFTPTEK |
| | | DEYACRVNHVTLSQPKIVKWDRDM |
| 12 | HaloTag ®-B2M- | MSRSVALAVLALLSLSGLEAAEIGTGFPFDPHYVEVLGERMHYVDVG |
| | HLA-A02 fusion | PRDGTPVLFLHGNPTSSYVWRNIIPHVAPTHRCIAPDLIGMGKSDKPDL |
| | [SignalP]- | GYFFDDHVRFMDAFIEALGLEEVVLVIHDWGSALGFHWAKRNPERVK |
| | [HaloTag ®]-[B2M]- | GIAFMEFIRPIPTWDEWPEFARETFQAFRTTDVGRKLIIDQNVFIEGTLP |
| | [HLA-A02] | MGVVRPLTEVEMDHYREPFLNPVDREPLWRFPNELPIAGEPANIVALV |
| | | EEYMDWLHQSPVPKLLFWGTPGVLIPPAEAARLAKSLPNCKAVDIGP |
| | | GLNLLQEDNPDLIGSEIARWLSTLEISGGGGGSGGGGSGGGGSIQRTPK |
| | | IQVYSRHPAENGKSNFLNCYVSGFHPSDIEVDLLKNGERIEKVEHSDLS |
| | | FSKDWSFYLLYYTEFTPTEKDEYACRVNHVTLSQPKIVKWDRDMGGG |
| | | GSGGGGSGGGGSGGGGSGSHSMRYFFTSVSRPGRGEPRFIAVGYVDD |
| | | TQFVRFDSDAASQRMEPRAPWIEQEGPEYWDGETRKVKAHSQTHRV |
| | | DLGTLRGYYNQSEAGSHTVQRMYGCDVGSDWRFLRGYHQYAYDGK |
| | | DYTALKEDLRSWTAADMAAQTTKHKWEAAHVAEQLRAYLEGTCVE |
| | | WLRRYLENGKETLQRTDAPKTHMTHHAVSDHEATLRCWALSFYPAEI |
| | | TLTWQRDGEDQTQDTELVETRPAGDGTFQKWAAVVVPSGQEQRYTC |
| | | HVQHEGLPKPLTLRWEPSSQPTIPIVGIIAGLVLFGAVITGAVVAAVMW |
| | | RRKSSDRKGGSYSQAASSDSAQGSDVSLTACKV |
| 13 | Mature HaloTag ®- | AEIGTGFPFDPHYVEVLGERMHYVDVGPRDGTPVLFLHGNPTSSYVW |
| | B2M-HLA-A02 | RNIIPHVAPTHRCIAPDLIGMGKSDKPDLGYFFDDHVREMDAFIEALGL |
| | fusion (lacking | EEVVLVIHDWGSALGFHWAKRNPERVKGIAFMEFIRPIPTWDEWPEFA |
| | signal peptide) | RETFQAFRTTDVGRKLIIDQNVFIEGTLPMGVVRPLTEVEMDHYREPFL |
| | [HaloTag ®]-[B2M]- | NPVDREPLWRFPNELPIAGEPANIVALVEEYMDWLHQSPVPKLLFWG |
| | [HLA-A02] | TPGVLIPPAEAARLAKSLPNCKAVDIGPGLNLLQEDNPDLIGSEIARWL |
| | | STLEISGGGGGSGGGGSGGGGSIQRTPKIQVYSRHPAENGKSNFLNCY |
| | | VSGFHPSDIEVDLLKNGERIEKVEHSDLSFSKDWSFYLLYYTEFTPTEK |
| | | DEYACRVNHVTLSQPKIVKWDRDMGGGGSGGGGSGGGGSGGGGSGS |
| | | HSMRYFFTSVSRPGRGEPRFIAVGYVDDTQFVREDSDAASQRMEPRAP |
| | | WIEQEGPEYWDGETRKVKAHSQTHRVDLGTLRGYYNQSEAGSHTVQ |
| | | RMYGCDVGSDWRFLRGYHQYAYDGKDYIALKEDLRSWTAADMAAQ |
| | | TTKHKWEAAHVAEQLRAYLEGTCVEWLRRYLENGKETLQRTDAPKT |
| | | HMTHHAVSDHEATLRCWALSFYPABITLTWQRDGEDQTQDTELVETR |
| | | PAGDGTFQKWAAVVVPSGQEQRYTCHVQHEGLPKPLTLRWEPSSQPT |
| | | IPIVGIIAGLVLFGAVITGAVVAAVMWRRKSSDRKGGSYSQAASSDSA |
| | | QGSDVSLTACKV |
| 14 | NY-ESO-1 antigen | SLLMWITQC |
| | peptide | |
| 15 | WT-1 antigen | RMFPNAPYL |
| | peptide | |
| 16 | MART-1 antigen | ELAGIGILTV |
| | peptide | |
| 17 | CMV antigen | NLVPMVATV |
| | peptide | |
| 18 | ssDNA oligo | CGGAGATGTGTATAAGAGACAGNNNNNNNNNNGGACGCAACTTAA |
| | barcode consensus | GANNNNNNNNNCCCATATAAGAAA |
| | sequence | wherein ‘N’ = A, C, G or T |
| 19 | S. aureus sortase A | MKKWTNRLMTIAGVVLILVAAYLFAKPHIDNYLHDKDKDEKIEQYDK |
| | (WT) | NVKEQASKDKKQQAKPQIPKDKSKVAGYIEIPDADIKEPVYPGPATSE |
| | | QLNRGVSFAEENESLDDQNISIAGHTFIDRPNYQFTNLKAAKKGSMVY |
| | | FKVGNETRKYKMTSIRDVKPTDVQVLDEQKGKDKQLTLITCDDYNEK |
| | | TGVWEKRKIFVATEVK |
| 20 | S. aureus sortase A | QAKPQIPKDKSKVAGYIEIPDADIKEPVYPGPATSEQLNRGVSFAEENE |
| | sortase domain (WT) | SLDDQNISIAGHTFIDRPNYQFTNLKAAKKGSMVYFKVGNETRKYKM |
| | | TSIRDVKPTDVGVLDEQKGKDKQLTLITCDDYNEKTGVWEKRKIFVA |
| | | TEVK |
| 21 | S. aureus sortase A | MKKWTNRLMTIAGVVLILVAAYLFAKPHIDNYLHDKDKDEKIEQYDK |
| | (K196T) | NVKEQASKDKKQQAKPQIPKDKSKVAGYIEIPDADIKEPVYPGPATSE |
| | | QLNRGVSFAEENESLDDQNISIAGHTFIDRPNYQFTNLKAAKKQSMVY |
| | | FKVGNETRKYKMTSIRDVKPTDVGVLDEQKGKDKQLTLITCDDYNEK |
| | | TGVWETRKIFVATEVK |
| 22 | S. aureus sortase A | QAKPQIPKDKSKVAGYIEIPDADIKEPVYPGPATSEQLNRQVSFAEENE |
| | sortase domain | SLDDQNISIAGHTFIDRPNYQFTNLKAAKKGSMVYFKVGNETRKYKM |
| | (K196T) | TSIRDVKPTDVGVLDEQKGKDKQLTLITCDDYNEKTGVWETRKIFVA |
| | | TEVK |
| 23 | S. pyogenes sortase | MVKKQKRRKIKSMSWARKLLIAVLLILGLALLENKSIRNTLIARNSNK |
| | | YQVTKVSKKQIKKNKEAKSTFDFQAVEPVSTESVLQAQMAAQQLPVI |
| | | GGIAIPELGINLPIFKGLGNTELIYGAGTMKEEQVMGGENNYSLASHHI |
| | | FGITGSSQMLFSPLERAQNQMSIYLTDKEKIYEYIIKDVFTVAPERVDVI |
| | | DDTAGLKEVTLVTCTDIEATERIIVKGELKTEYDFDKAPADVLKAFNH |
| | | SYNQVST |
| 24 | S. pyogenes sortase | VLQAQMAAQQLPVIGGIAIPELGINLPIFKGLGNTELIYGAGTMKEEQV |
| | A sortase domain | MGGENNYSLASHHIFGITGSSQMLFSPLERAQNGMSIYLTDKEKIYEYI |
| | | IKDVFTVAPERVDVIDDTAGLKEVTLVTCTDIEATERIIVKGELKTEYD |
| | | FDKAPADVLKAFNHSYNQVST |
| 25 | Sortase substrate | LX₁X₂TX₂ |
| | motif CON | wherein X₂ = P, A or S, X₂ = any amino acid, and X₃ = G, |
| | | A, S or C |
| 26 | Sortase substrate | LPX₁TG |
| | motif 1 | wherein X₁ = any amino acid |
| 27 | Sortase substrate | LPX₁TA |
| | motif 2 | wherein X₁ = any amino acid |
| 28 | Sortase substrate | LPX₁TS |
| | motif 3 | wherein X₁ = any amino acid |
| 29 | anti-DOTAM IgG | VTLKESGPVLVKPTETLTLTCTVSGFSLSTYSMSWIRQPPGKALEWLG |
| | HC | FIGSRGDTYYASWAKGRLTISKDTSKSQVVLTMTNMDPVDTATYYCA |
| | | RERDPYGGGAYPPHLWGRGTLVTVSSASTKGPSVFPLAPSSKSTSGGT |
| | | AALGCLVKDYFPEPVTVSWNSGALTSGVHTFPAVLQSSGLYSLSSVVT |
| | | VPSSSLGTQTYICNVNHKPSNTKVDKKVEPKSCDKTHTCPPCPAPELL |
| | | GGPSVFLFPPKPKDTLMISRTPEVTCVVVDVSHEDPEVKFNWYVDGVE |
| | | VHNAKTKPREEQYNSTYRVVSVLTVLHQDWLNGKEYKCKVSNKALP |
| | | APIEKTISKAKGQPREPQVYTLPPSRDELTKNQVSLTCLVKGFYPSDIA |
| | | VEWESNGQPENNYKTTPPVLDSDGSFFLYSKLTVDKSRWQQGNVFSC |
| | | SVMHHALHNHYTQKSLSLSPGK |
| 30 | anti-DOTAM Fab | QVTLKESGPVLVKPTETLTLTCTVSGFSLSTYSMSWIRQPPGKALEWL |
| | HC sortA | GFIGSRGDTYYASWAKGRLTISKDTSKSQVVLTMTNMDPVDTATYYC |
| | | ARERDPYGGGAYPPHLWGRGTLVTVSSASTKGPSVFPLAPSSKSTSGG |
| | | TAALGCLVKDYFPEPVTVSWNSGALTSGVHTFPAVLQSSGLYSLSSVV |
| | | TVPSSSLGTQTYICNVNHKPSNTKVDKKVEPKSCGGGGSGGGGSGGG |
| | | GSGGGGSQAKPQIPKDKSKVAGYIEIPDADIKEPVYPGPATSEQLNRGV |
| | | SFAEENESLDDQNISIAGHTFIDRPNYQFTNLKAAKKGSMVYFKVGNE |
| | | TRKYKMTSIRNVKPTDVGVLDEQKGKDKQLTLITCDDYNEKTGVWE |
| | | TRKIFVATEVK |
| 31 | anti-DOTAM IgG | SIQMTQSPSSLSASVGDRVTITCQSSHSVYSDNDLAWYQQKPGKAPKL |
| | LC | LIYQASKLASGVPSRFSGSGSGTDFTLTISSLQPEDFATYYCLGGYDDE |
| | | SDTYGFGGGTKVEIKRTVAAPSVFIFPPSDEQLKSGTASVVCLLNNFYP |
| | | REAKVQWKVDNALQSGNSQESVTEQDSKDSTYSLSSTLTLSKADYEK |
| | | HKVYACEVTHQGLSSPVTKSFNRGEC |
| 32 | anti-DOTAM Fab | DIQMTQSPSSLSASVGDRVTITCQSSHSVYSDNDLAWYQQKPGKAPKL |
| | LC | LIYQASKLASGVPSRFSGSGSGTDFTLTISSLQPEDFATYYCLGGYDDE |
| | | SDTYGFGGGTKVEIKRTVAAPSVFIFPPSDEQLKSGTASVVCLLNNFYP |
| | | REAKVQWKVDNALQSGNSQESVTEQDSKDSTYSLSSTLTLSKADYEK |
| | | HKVYACEVTHQGLSSPVTKSFNRGEC |
| 33 | anti-DOTAM VH | VTLKESGPVLVKPTETLTLTCTVSGFSLSTYSMSWIRQPPGKALEWLG |
| | | FIGSRGDTYYASWAKGRLTISKDTSKSQVVLTMTNMDPVDTATYYCA |
| | | RERDPYGGGAYPPHLWGRGTLVTVSS |
| 34 | anti-DOTAM HC- | GFSLSTYSMS |
| | CDR1 | |
| 35 | anti-DOTAM HC- | FIGSRGDTYYASWAKG |
| | CDR2 | |
| 36 | anti-DOTAM HC- | ERDPYGGGAYPPHL |
| | CDR3 | |
| 37 | anti-DOTAM HC- | VTLKESGPVLVKPTETLTLTCTVS |
| | FR1 | |
| 38 | anti-DOTAM HC- | WIRQPPGKALEWLG |
| | FR2 | |
| 39 | anti-DOTAM HC- | RLTISKDTSKSQVVLTMINMDPVDTATYYCAR |
| | FR3 | |
| 40 | anti-DOTAM HC- | RGTLVTVSS |
| | FR4 | |
| 41 | anti-DOTAM VL | IQMTQSPSSLSASVGDRVTITCQSSHSVYSDNDLAWYQQKPGKAPKLL |
| | | IYQASKLASGVPSRFSGSGSGTDFTLTISSLQPEDFATYYCLGGYDDES |
| | | DTYGFGGGTKVEIK |
| 42 | anti-DOTAM LC- | QSSHSVYSDNDLA |
| | CDR1 | |
| 43 | anti-DOTAM LC- | QASKLAS |
| | CDR2 | |
| 44 | anti-DOTAM LC- | LGGYDDESDTYG |
| | CDR3 | |
| 45 | anti-DOTAM LC- | IQMTQSPSSLSASVGDRVTITC |
| | FR1 | |
| 46 | anti-DOTAM LC- | WYQQKPGKAPKLLIY |
| | FR2 | |
| 47 | anti-DOTAM LC- | GVPSRFSGSGSGTDFTLTISSLQPEDFATYYC |
| | FR3 | |
| 48 | anti-DOTAM LC- | FGGGTKVEIK |
| | FR4 | |

### Numbered Paragraphs

The following numbered paragraphs (paras) provide further statements of features and combinations of features which are contemplated in connection with the present disclosure.
1A. A polypeptide comprising (i) the amino acid sequence of a major histocompatibility complex (MHC) polypeptide, and (ii) a moiety facilitating labelling of the polypeptide with an identifier moiety.
2A. The polypeptide according to para IA. wherein the MHC polypeptide is β2 microglobulin.
3A. The polypeptide according to para 1A or para 2A. wherein the identifier moiety is a nucleic acid moiety
4A. The polypeptide according to any one of paras 1 A to 3A, wherein the identifier moiety
15 comprises or consists of single-stranded DNA (ssDNA).
5A. The polypeptide according to any one of paras 1A to 4A. wherein the moiety facilitating labelling of the polypeptide with an identifier moiety is or comprises a self-labelling protein tag.
6A. The polypeptide according to any one of paras 1A to 5A, wherein the moiety facilitating labelling of the polypeptide with an identifier moiety is or comprises a HaloTag®.
7A. A polypeptide comprising (i) the amino acid sequence of β2 microglobulin, and (ii) HaloTag®.
8A The polypeptide according to any one of paras 1A to 7A. further comprising a detectable moiety.
9A. The polypeptide according to para 8A, wherein the detectable moiety is a fluorescent label
10A. The polypeptide according to any one of paras 1 A to 9A, wherein the polypeptide comprises or consists of an amino acid sequence having at least 70% amino acid sequence identity to SEQ ID NO: 1 1, 10. 13 or 12.
11A. A polypeptide comprising (i) the amino acid sequence of a major histocompatibility complex (MIC) polypeptide, and (ii) an identifier moiety. w herein the identifier moiety is covalently associated with the polypeptide via linkage formed by a self-labelling protein tag.
12A. The polypeptide according to para 11A, wherein the MHC polypeptide is β2 microglobulin.
13A The polypeptide according to para 11 A or para 12A, wherein the identifier moiety is a nucleic acid moiety.
14A. The polypeptide according to any one of paras 11A to 13A, wherein the identifier moiets comprises or consists of single-stranded DNA (ssDNA )
15A. The polypeptide according to any one of paras 11 A to 14A, wherein the self-labelling protein tag is or comprises a HaloTag®.
16A. The polypeptide according to para 1SA. wherein the identifier moiety is covalently associated with the polypeptide via an ester bond formed by dehalogenase activity of the HaloTag®; on a HaloTag® ligand comprising the identifier moiety and a chloroalkane moiety
17A. A polypeptide comprising (i) the amino acid sequence of D2 microglobulin, (ii) a HaloTag®, and (iii) a single-stranded DNA (ssDNA) moiety linked to the polypeptide via an ester bond formed by dehalogenase activity of the HaloTag® on a HaloTag®, ligand comprising the ssDNA moiety and a chloroalkane moiety.
18A. The polypeptide according to any one of paras 11A to 17A, further comprising a detectable moiety.
19A. The polypeptide according to para 18A. wherein the detectable moiety is a fluorescent label.
20A. An MHC molecule comprising a polypeptide according to any one of paras 1A to 19A.
21A. An MHC peptide complex, comprising an MHC molecule according to para 20A, and a peptide presented by the MHC molecule
22A. A nucleic acid, or a plurality of nucleic acids, encoding a polypeptide according to any one of paras 1A to 10A.
23A. The nucleic acid or plurality of nucleic acids according to para 11A or para 12A, further comprising nucleic acid encoding a peptide presented by an MHC molecule comprising a polypeptide according to any one of paras 1 A to 10A.
24A. An expression vector, or a plurality of expression vectors, comprising a nucleic acid, or a plurality of nucleic acids, according to para 22A or para 23A.
25A. A cell comprising a polypeptide according to any one of paras 1A to 19A, an MHC molecule according to para 20A, an MHC:peptide complex according to para 21A, a nucleic acid or plurality of nucleic acids according to any one of paras 21 A to 23A, or an expression vector or plurality of expression vectors according to para 24A.
26A. The cell according to para 15A, wherein the cell is an antigen presenting cell (APC),
27A. A method for producing a cell comprising an MHC molecule labelled with an identifier moiety, comprising:
   (1) introducing into a cell a nucleic acid or plurality of nucleic acids according to para 35 22A; and
   (2) contacting the cell with a labelling moiety comprising an identifier moiety. wherein the labelling moiety is suitable for labelling the polypeptide encoded by the nucleic acid or plurality of nucleic acids of (1) with an identifier moiety.
28A. A method for producing a cell comprising an MHC molecule labelled with a single-stranded DNA (ssDNA) moiety, comprising
   (1) introducing into a cell a nucleic acid or plurality of nucleic acids encoding a polypeptide comprising (i) the amino acid sequence of β2 microglobulin, and (ii) a HaloTag; and
   (2) contacting the cell with a HaloTag® ligand comprising a ssDNA moiety and a chloroalkane moiety
29A. A method for producing a cell comprising an MHC:peptide complex comprising an MHC molecule labelled with an identifier moiety. comprising:
   (1) introducing into a cell a nucleic acid or plurality of nucleic acids according to para 22A.
   (2) introducing into the cell. (i) a peptide presented by an MHC molecule comprising a polypeptide encoded by the nucleic acid or plurality of nucleic acids of (1), or (ii) nucleic acid encoding a peptide presented by an MHC molecule comprising a polypeptide encoded by the nucleic acid or plurality of nucleic acids of(1); and
   (3) contacting the cell with a labelling moiety comprising an identifier moiety. wherein the labelling moiety is suitable for labelling the polypeptide encoded by the nucleic acid or plurality of nucleic acids of (1) with the identifier moiety via the moiety facilitating labelling of the polypeptide with an identifier moiety.
30A. A method for producing a cell comprising an MHC:peptide complex comprising an MHC molecule labelled with a single-stranded DNA (ssDNA) moiety. comprising:
   (1) introducing into a cell a nucleic acid or plurality of nucleic acids encoding a polypeptide comprising (i) the amino acid sequence of β2 microglobulin, and (ii) a HaloTag®,
   (2) introducing into the cell: (i) a peptide presented by an MHC molecule comprising the polypeptide of (1). or (ii) nucleic acid encoding a peptide presented by an MHC molecule comprising the polypeptide of (I) and
   (3) contacting the cell with a HaloTag® ligand comprising the ssDNA moiety and a chloroalkane moiety
31A. A cell produced by the method according to any one of paras 27A to 30A.
32A. A composition comprising a cell according to any one of paras 25A, 26A or 31A, and a T cell.
33A. A method for identifying a T cell receptor (TCR) that binds to an MHC:peptide complex. comprising:
   (1) contacting a cell comprising an MHC:peptide complex according to para 21 A with a population of T cells:
   (2) incubating the cells under conditions suitable for trogocytosis of an MHC:peptide complex by a T cell comprising a TCR that binds to the MHC:peptide complex; and
   (3) subsequently analysing T cells to identify a TCR that binds to the MHC:peptide complex
34A. A kit, comprising:
   (1) a nucleic acid or plurality of nucleic acids according to para 22A, and
   (2) a labelling moiety comprising an identifier moiety. wherein the labelling moiety is suitable for labelling the polypeptide encoded by the nucleic acid or plurality of nucleic acids according to (1) with the identifier moiety via the moiety facilitating labelling of the polypeptide with an identifier moiety
35A. A kit, comprising:
   (1) a nucleic acid, or a plurality of nucleic acids, encoding a polypeptide comprising (i) the amino acid sequence of β2 microglobulin, and (ii) a HaloTag® and
   (2) a HaloTag® ligand comprising a ssDNA moiety and a chloroalkane moiety.
36A The kit according to para 35A or para 36A. further comprising:
   (3) a peptide presented by an MHC molecule comprising a polypeptide encoded by the nucleic acid or plurality of nucleic acids according to (1). or nucleic acid encoding a peptide presented by an MHC molecule comprising a polypeptide encoded by the nucleic acid or plurality of nucleic acids according to (1),
37A. A kit, comprising:
   (1) a cell comprising an M-IC molecule according to para 20A, or a plurality of such cells, and
   (2) a peptide presented by an MHC molecule of a cell according to (1). or nucleic acid encoding a peptide presented by an MHC molecule of a cell according to (1)
38A. A kit, comprising a cell comprising an MHC:peptide complex according to para 21A. or a plurality of such cells.
1B. A polypeptide comprising (i) the amino acid sequence of a major histocompatibility complex (MHC) polypeptide., and (ii) a moiety facilitating labelling of the polypeptide with an identifier moiety.
2B. The polypeptide according to para B, wherein the MHC polypeptide is β2 microglobulin.
3B. The polypeptide according to para 1B or para 2B, wherein the identifier moiety is a nucleic acid moiety.
4B. The polypeptide according to any one of paras 1B to 3B, wherein the identifier moiety comprises or consists of single-stranded DNA (ssDNA)
5B. The polypeptide according to any one of paras 1B to 48, wherein the moiety facilitating labelling of the polypeptide with an identifier moiety is or comprises a self-labelling protein tag.
6B. The polypeptide according to any one of paras 1 B to 5B, wherein the moiety facilitating labelling of the polypeptide with an identifier moiety is or comprises a HaloTag®.
7B. The polypeptide according to any one of paras 1B to 6B. wherein the polypeptide further comprises a sortase substrate motif.
8B. A polypeptide comprising (i) the amino acid sequence of β2 microglobulin, and (ii) HaloTag®.
9B. A polypeptide comprising (i) the amino acid sequence of β2 microglobulin, (ii) a sortase substrate motif, and (iii) HaloTag®.
10B. The polypeptide according to any one of paras 1B to 9B, wherein the polypeptide further comprises a detectable moiety.
11 B. The polypeptide according to para 10B, wherein the detectable moiety is a fluorescent label.
12B. The polypeptide according to any one of paras 1B to 11B. wherein the polypeptide comprises or consists of an amino acid sequence having at least 7013% amino acid sequence identity to SEQ ID NO 11, 10. 13 or 12.
13B. A polypeptide comprising (i) the amino acid sequence of a major histocompatibility complex (MHC) polypeptide, and (ii) an identifier moiety, wherein the identifier moiety is covalently associated with the polypeptide via linkage formed by a self-labelling protein tag.
14B. The polypeptide according to para 13B, wherein the MHC polypeptide is β2 microglobulin.
15B. The polypeptide according to para 13B or para 14B, wherein the identifier moiety is a nucleic acid moiety.
16B. The polypeptide according to any one of paras 13B to 15B, wherein the identifier moiety comprises or consists of single-stranded DNA (ssDNA),
17B. The polypeptide according to any one of paras 13B to 16B. wherein the self-labelling protein tag is or comprises a HaloTag®.
18B. The polypeptide according to para 17B, wherein the identifier moiety is covalently associated with the polypeptide via an ester bond formed by dehalogenase activity of the HaloTag® on a HaloTag ‘ligand comprising the identifier moiety and a chloroalkane moiety.
19B. The polypeptide according to any one of paras 13B to 18B. wherein the polypeptide further comprises a sortase substrate motif
20B. A polypeptide comprising (i) the amino acid sequence of β2 microglobulin, (ii) a HaloTag-, and (iii) a single-stranded DNA (ssDNA) moiety linked to the polypeptide via an ester bond formed by dehalogenase activity of the HaloTag® on a HaloTag t ligand comprising the ssDNA moiety and a chloroalkane moiety.
21B. A polypeptide comprising (i) the amino acid sequence of β2 microglobulin, (ii) a sortase substrate motif, (iii) a HaloTag, and (iv) a single-stranded DNA (ssDNA) moiety linked to the polypeptide via an ester bond formed by dehalogenase activity of the HaloTag® on a HaloTag® ligand comprising the ssDNA moiety and a chloroalkane moiety.
22B. The polypeptide according to any one of paras 12B to 21B. wherein the polypeptide further comprises a detectable moiety.
23B. The polypeptide according to para 22B., wherein the detectable moiety is a fluorescent label.
24B. A MHC molecule comprising a polypeptide according to any one of paras 1 B to 23B
25B. A MHC:peptide complex. comprising a MHC molecule according to para 24B, and a peptide presented by the MHC molecule.
26B. A nucleic acid, or a plurality of nucleic acids, encoding a polypeptide according to any one of paras 1B to 12B.
27B. The nucleic acid or plurality of nucleic acids according to para 26B. wherein the nucleic acid or plurality of nucleic acids further comprises nucleic acid encoding a peptide presented by a MHC molecule comprising a polypeptide according to any one of paras 11B to 12B.
28B. An expression vector, or a plurality of expression vectors, comprising a nucleic acid, or a plurality of nucleic acids, according to para 26B or para 27B.
29B. A cell comprising a polypeptide according to any one of paras 1B to 23B, a MHC molecule according to para 24B, a MHC:peptide complex according to para 25B, a nucleic acid or plurality of nucleic acids according to para 26B or para 27B, or an expression vector or plurality of expression vectors according to para 28B.
30B. The cell according to para 29B, wherein the cell is an antigen presenting cell (APC),
31B. A method for producing a cell comprising a MHC molecule labelled with an identifier moiety. comprising.
   (1) introducing into a cell a nucleic acid or plurality of nucleic acids according to para 26B; and
   (2) contacting the cell with a labelling moiety comprising an identifier moiety, herein the labelling moiety is suitable for labelling the polypeptide encoded by the nucleic acid or plurality of nucleic acids of (1) with an identifier moiety.
32B. A method for producing a cell comprising a MHC molecule labelled with a single-stranded DNA (ssDNA) moiety, comprising
   (1) introducing into a cell a nucleic acid or plurality of nucleic acids encoding a polypeptide comprising (i) the amino acid sequence of D2 microglobulin, and (ii) a HaloTag i. and
   (2) contacting the cell with a HaloTag® ligand comprising a ssDNA moiety and a chloroalkane moiety
33B. A method for producing a cell comprising a MHC molecule labelled with a single-stranded DNA (ssDNA) moiety, comprising
   (1) introducing into a cell a nucleic acid or plurality of nucleic acids encoding a polypeptide comprising (i) the amino acid sequence of D2 microglobulin, (ii) a sortase substrate motif, and (iii) a HaloTag®; and
   (2) contacting the cell with a HaloTag® ligand comprising a ssDNA moiety and a chloroalkane moiety
34B. A method for producing a cell comprising a MHC:peptide complex comprising a MHC molecule labelled with an identifier moiety, comprising
   (1) introducing into a cell a nucleic acid or plurality of nucleic acids according to para 26B;
   (2) introducing into the cell: (i) a peptide presented by a MHC molecule comprising a polypeptide encoded by the nucleic acid or plurality of nucleic acids of (1), or (ii) nucleic acid encoding a peptide presented by a MHC molecule comprising a polypeptide encoded by the nucleic acid or plurality of nucleic acids of (l); and
   (3) contacting the cell with a labelling moiety comprising an identifier moiety. wherein the labelling moiety is suitable for labelling the polypeptide encoded by the nucleic acid or plurality of nucleic acids of (1) with the identifier moiety via the moiety facilitating labelling of the polypeptide with an identifier moiety.
35B. A method for producing a cell comprising a MHC:peptide complex comprising a MHC molecule labelled with a single-stranded DNA (ssDNA) moiety, comprising:
   (1) introducing into a cell a nucleic acid or plurality of nucleic acids encoding a polypeptide comprising (i) the amino acid sequence of β2 microglobulin, and (ii) a HaloTag®,
   (2) introducing into the cell: (i) a peptide presented by a MHC molecule comprising the polypeptide of (1). or (ii) nucleic acid encoding a peptide presented by a MHC molecule comprising the polypeptide of (1); and
   (3) contacting the cell with a HaloTag® ligand comprising the ssDNA moiety and a chloroalkane moiety.
36B. A method for producing a cell comprising a MHC:peptide complex comprising a MHC molecule labelled with a single-stranded DNA (ssDNA) moiety. comprising:
   (1) introducing into a cell a nucleic acid or plurality of nucleic acids encoding a polypeptide comprising (i) the amino acid sequence of β2 microglobulin, (ii) a sortase substrate motif, and iii) a HaloTag®;
   (2) introducing into the cell. (i) a peptide presented by a MHC molecule comprising the polypeptide of (1), or (ii) nucleic acid encoding a peptide presented by a MHC molecule comprising the polypeptide of (1); and
   (3) contacting the cell with a HialoTag® ligand comprising the ssDNA moiety and a chloroalkane moiety
37B. A cell produced by the method according to any one of paras 31B to 36B.
38B. A composition comprising a cell according to any one of paras 29B. 30B or 37B, and a T cell.
39B. A method for identifying a T cell receptor (TCR) that binds to a MHC:peptide complex., comprising:
   (1) contacting (a) a cell comprising a MHC:peptide complex with (b) a population of T cells;
   (2) incubating the cells obtained after step (1) under conditions suitable for trogocytosis of a MHC:peptide complex by a T cell comprising a TCR that binds to the MHC-peptide complex; and
   (3) subsequently analysing the cells obtained after step (2) to identify a TCR that binds to the MHC peptide complex,
   wherein the MHC:peptide complex comprises a MHC molecule, and wherein the MHC molecule comprises a polypeptide comprising (i) the amino acid sequence of a MHC polypeptide, and (ii) an identifier moiety, wherein the identifier moiety is covalently associated with the polypeptide via linkage formed by a self-labelling protein tag.
40B. A method for identifying a T cell receptor (TCR) that binds to a MHC:peptide complex, comprising:
   (1) contacting (a) a cell comprising a MHC:peptide complex with (b) a population of cells comprising T cells comprising a polypeptide comprising a sortase acceptor motif at the cell surface, in the presence of a sortase;
   (2) incubating the cells obtained after step (1) under conditions suitable for interaction between the cell of (α) and the population of cells of (b); and
   (3) subsequently analysing the cells obtained after step (2) to identify a T cell comprising a TCR that binds to the MHC:peptide complex:
   wherein the MHC:peptide complex comprises a MHC molecule, and wherein the MHC molecule comprises a polypeptide comprising (i) the amino acid sequence of a MHC polypeptide. (ii) a sortase substrate motif, and (iii) an identifier moiety. wherein the identifier moiety is covalently associated with the polypeptide via linkage formed by a self-labelling protein tag.
41B. The method according to para 40B. wherein the sortase is provided at the cell surface of T cells comprising a polypeptide comprising a sortase acceptor motif.
42B. The method according to any one of paras 39B to 41B, wherein the MHC: polypeptide is β2 microglobulin.
43B. The method according to any one of paras 39B to 42B, wherein the identifier moiety is a nucleic acid moiety.
44B. The method according to any one of paras 39B to 43B, wherein the identifier moiety comprises or consists of single-stranded DNA (ssDNA )
45B. The method according to any one of paras 39B to 44B, wherein the self-labelling protein tag is or comprises a HaloTag®.
46B. The method according to para 45B, wherein the identifier moiety is covalently associated with the polypeptide via an ester bond formed by dehalogenase activity of the HaloTag® on a HaloTag® % ligand comprising the identifier moiety and a chloroalkane moiety.
47B. The method according to any one of paras 39B to 4613, wherein the polypeptide comprising the amino acid sequence of a MHC polypeptide and an identifier moiety further comprises a detectable moiety.
48B. The method according to para 47B, wherein the detectable moiety is a fluorescent label
49B. A kit, comprising:
   (1) a nucleic acid or plurality of nucleic acids according to para 26B, and
   (2) a labelling moiety comprising an identifier moiety, wherein the labelling moiety is suitable for labelling the polypeptide encoded by the nucleic acid or plurality of nucleic acids according to (1) with the identifier moiety via the moiety facilitating labelling of the polypeptide with an identifier moiety.
50B. A kit. comprising:
   (1) a nucleic acid, or a plurality of nucleic acids, encoding a polypeptide comprising (i) the amino acid sequence of β2 microglobulin, and (ii) a HaloTag®, and
   (2) a HaloTag® ligand comprising a ssDNA moiety and a chloroalkane moiety
51 B. A kit, comprising.
   (1) a nucleic acid, or a plurality of nucleic acids. encoding a polypeptide comprising (i) the amino acid sequence of D2 microglobulin, (ii) a sortase substrate motif, and (iii) a HaloTag®; and
   (2) a HaloTag®.- ligand comprising a ssDNA moiety and a chloroalkane moiety
52B. The kit according to para 51B. wherein the kit further comprises:
   a sortase, and reagents for modifying cells to comprise a polypeptide comprising a sortase acceptor motif at the cell surface.
53B. The kit according to para 51B or para 52B, wherein the kit comprises reagents for modifying cells to comprise a sortase at the cell surface.
54B. The kit according to any one of paras 49B to 53B. wherein the kit further comprises a peptide presented by a MHC molecule comprising a polypeptide encoded by the nucleic acid or plurality of nucleic acids according to (1). or nucleic acid encoding a peptide presented by a MHC molecule comprising a polypeptide encoded by the nucleic acid or plurality of nucleic acids according to (1),
55B. A kit, comprising:
   (1) a cell comprising a MHC molecule according to para 24B. or a plurality of such cells. and
   (2) a peptide presented by a MHC molecule of a cell according to (1), or nucleic acid encoding a peptide presented by a MHC molecule of a cell according to (1),
56B. A kit, comprising a cell comprising a MHC:peptide complex according to para 25B, or a plurality of such cells.
57B. A polypeptide comprising (i) a sortase substrate motif, and (ii) a moiety facilitating labelling of the polypeptide with an identifier moiety.
58B. The polypeptide according to para 57B. wherein the polypeptide further comprises the amino acid sequence of a protein that localises to the cell membrane
59B. The polypeptide according to any one of paras 57B to 58B. wherein the moiety facilitating labelling of the polypeptide with an identifier moiety is or comprises a self-labelling protein tag.
608. The polypeptide according to any one of paras 57B to 59B, wherein the moiety facilitating labelling of the polypeptide with an identifier moiety is or comprises a HaloTag.
61B. The polypeptide according to any one of paras 57B to 60B. wherein the identifier moiety is a nucleic acid moiety.
62B. The polypeptide according to any one of paras 57B to 61B, wherein the identifier moiety comprises or consists of single-stranded DNA (ssDNA)
63B. A polypeptide comprising (i) the amino acid sequence of a protein that localises to the cell membrane, (ii) a sortase substrate motif and (iii) a HaloTag®.
64B. The polypeptide according to any one of paras 578 to 63B, wherein the polypeptide further comprises a detectable moiety.
65B. The polypeptide according to para 648, wherein the detectable moiety is a fluorescent label.
66B. A polypeptide comprising (i) a sortase substrate motif, and (ii) an identifier moiety, wherein the identifier moiety is covalently associated with the polypeptide via linkage formed by a self-labelling protein tag.
67B. The polypeptide according to any one of paras 63B to 66B. wherein the self-labelling protein tag is or comprises a HaloTag®.
68B. The polypeptide according to para 67B, wherein the identifier moiety is covalently associated with the polypeptide via an ester bond formed by dehalogenase activity of the HaloTag® on a HaloTag®, ligand comprising the identifier moiety and a chloroalkane moiety
69B. The polypeptide according to any one of paras 63B to 68B, wherein the identifier moiety is a nucleic acid moiety
70B. The polypeptide according to any one of paras 63B to 68B. wherein the identifier moiety comprises or consists of single-stranded DNA (ssDNA),
71B. A polypeptide comprising (i) the amino acid sequence of a protein that localises to the cell membrane. (ii) a sortase substrate motif, (iii) a HaloTag®, and (iv) a single-stranded DNA (ssDNA) moiety linked to the polypeptide via an ester bond formed by dehalogenase activity of the HaloTag®; on a HaloTag® ligand comprising the ssDNA moiety and a chloroalkane moiety
72B. The polypeptide according to any one of paras 63B to 71B, wherein the polypeptide further comprises a detectable moiety.
73B. The polypeptide according to para 72B, wherein the detectable moiety is a fluorescent label.
74B. A nucleic acid, or a plurality of nucleic acids, encoding a polypeptide according to any one of paras 57B to 658.
75B. An expression vector, or a plurality of expression vectors, comprising a nucleic acid, or a plurality of nucleic acids, according to para 74B
76B. A cell comprising a polypeptide according to any one of paras 57B to 71B, a nucleic acid or plurality of nucleic acids according to para 74B, or an expression vector or plurality of expression vectors according to para 75B.
77B. The cell according to para 76B, wherein the cell is an antigen presenting cell (APC),
78B. A method for producing a cell comprising a polypeptide labelled with an identifier moiety. comprising:
   (1) introducing into a cell a nucleic acid or plurality of nucleic acids according to para 74B, and
   (2) contacting the cell with a labelling moiety comprising an identifier moiety, wherein the labelling moiety is suitable for labelling the polypeptide encoded by the nucleic acid or plurality of nucleic acids of (1) with an identifier moiety.
79B. The method according to para 78B. wherein the identifier moiety is a nucleic acid moiety.
80B. The method according to paras 78B or para 79B, w herein the identifier moiety comprises or consists of single-stranded DNA (ssDNA),
81B. The method according to any one of paras 78B to 80B, wherein the moiety facilitating labelling of the polypeptide with an identifier moiety is or comprises a HaloTag,, and wherein the labelling moiety is a HaloTag® ligand comprising an identifier moiety and a chloroalkane moiety.
82B. A method for producing a cell comprising a polypeptide labelled with a single-stranded DNA (ssDNA) moiety, comprising
   (1) introducing into a cell a nucleic acid, or a plurality of nucleic acids, encoding a polypeptide comprising (i the amino acid sequence of a protein that localises to the cell membrane, (ii) a sortase substrate motif, and (iii) a HaloTag®; and
   (2) contacting the cell with a HaloTag® ligand comprising a ssDNA moiety and a chloroalkane moiety.
83B. A cell produced by the method according to any one of paras 78B to 82B.
84B. A composition comprising:
   (a) a cell comprising a polypeptide according to any one of paras 57B to 73B at its cell surface.
   (b) a cell comprising a polypeptide comprising a sortase acceptor motif at its cell surface, and
   (c) a sortase.
85B. The composition according to para 843, wherein the composition comprises
   (a) a cell comprising a polypeptide according to any one of paras 57B to 73B at its cell surface, and
   (b) a cell comprising at its cell surface (i) α polypeptide comprising a sortase acceptor motif, and (ii) a sortase.
86B. The composition according to para 84B or para 85B. wherein the cell according to (a) is an antigen-presenting cell, and/or wherein the cell according to (b) is a T cell.
87B. A method for identifying a cell that has interacted with a cell comprising a polypeptide according to any one of paras 66B to 73B at its cell surface, comprising.
   (1) contacting (a) a cell comprising a polypeptide according to any one of paras 66B to
73B at its cell surface with (b) a population of cells comprising a cell comprising a polypeptide comprising a sortase acceptor motif at its cell surface, in the presence of a sortase; and
   (2) incubating the population of cells obtained after step (1) under conditions suitable for interaction between the cell of (α) and the population of cells of (b); and
   (3) subsequently analysing the cells obtained after step (2) to identify a cell within the population of cells of (b) that has undergone sortase-mediated transfer of an identifier moiety. thereby identifying a cell that has interacted with a cell comprising a polypeptide according to any one of paras 668 to 73B at its cell surface.
88B. The method according to para 87B, w herein the sortase is provided at the cell surface of the cell comprising a polypeptide comprising a sortase acceptor motif. 89B. A kit. comprising:
   (1) a nucleic acid or plurality of nucleic acids according to para 74B; and
   (2) a labelling moiety comprising an identifier moiety, wherein the labelling moiety is suitable for labelling the polypeptide encoded by the nucleic acid or plurality of nucleic acids according to (1) with the identifier moiety via the moiety facilitating labelling of the polypeptide with an identifier moiety.
90B. A kit. comprising.
   (1) a nucleic acid, or a plurality of nucleic acids. encoding a polypeptide comprising (i) the amino acid sequence of a protein that localises to the cell membrane, (ii) a sortase substrate motif, and (iii) a HaloTag®; and
   (2) a HaloTag® ligand comprising a ssDNA moiety and a chloroalkane moiety
91B. The kit according to para 89B or para 90., wherein the kit further comprises. a sortase, and reagents for modifying cells to comprise a polypeptide comprising a sortase acceptor motif at the cell surface.
92B. The kit according to any one of paras 89B to 91B, wherein the kit comprises reagents for modifying cells to comprise a sortase at the cell surface The present disclosure includes the combination of the aspects and preferred features described except where such a combination is clearly impermissible or expressly avoided.

The section headings used herein are for organisational purposes only and are not to be construed as limiting the subject matter described Aspects and embodiments of the present disclosure 6ill now be illustrated, by % ay of example. with reference to the accompanying figures. Further aspects and embodiments will be apparent to those skilled in the art. All documents mentioned in this text are incorporated herein by reference.

Throughout this specification, including the claims which follow, unless the context requires otherwise. the word ‘comprise,’ and variations such as ‘comprises' and ‘comprising,’ will be understood to imply the inclusion of a stated integer or step or group of integers or steps but not the exclusion of any other integer or step or group of integers or steps.

As used herein, a ‘peptide’ refers to a chain of two or more amino acid monomers linked by peptide bonds. A peptide typically has a length in the region of about 2 to 50 amino acids. A ‘polypeptide’ is a polymer chain of two or more peptides. Polypeptides typically have a length greater than about 50 amino acids. Reference herein to peptides, polypeptides and proteins also includes glycopeptides/glycopolypeptides/glycoproteins. lipopeptides/lipopolypeptides/lipoproteins, nucleopeptides/nucleopolypeptides.nucleoproteins, etc.

As used herein, an amino acid sequence, or a region of α polypeptide which -corresponds' to a specified reference amino acid sequence or region of α polypeptide has at least 60%, e g. one of at least ≥65%, ≥70%, ≥75%, ≥80%, ≥85%, ≥90%, ≥91%, ≥92%, ≥93%, ≥94%, ≥95%, ≥96%, ≥97%, ≥98%, ≥99% or ≥100% sequence identity to the amino acid sequence of the amino acid sequence/polypeptide/region An amino acid sequence/region/position of α polypeptide/amino acid sequence which ‘corresponds' to a specified reference amino acid sequence/region/position of a polypeptide/amino acid sequence can be identified by sequence alignment of the subject sequence to the reference sequence, e.g. using sequence alignment software such as ClustalOmega (Sding, J. 2005, Bioinformatics 21. 951-960).

As used herein, an amino acid sequence (e.g. the amino acid sequence of a peptide/polypeptide/domain/region) which is ‘derived from- a reference amino acid sequence (e.g. the amino acid sequence of a reference peptide/polypeptide/domain.regior) comprises. or consists of, an amino acid sequence having at least 60%. e.g. one of at least 65%., 70%, ≥75%, 80%, ≥85%, ≥90%, ≥91%, ≥92%, ≥93%, ≥94%, ≥95%, ≥96%, ≥97%, ≥98%, ≥99% or ≥100% sequence identity to the reference amino acid sequence.

It must be noted that, as used in the specification and the appended claims, the singular forms ‘a,’ ‘an,’ and ‘the’ include plural referents unless the context clearly dictates otherwise. Ranges may be expressed herein as from -about- one particular value., and/or to ‘about’ another particular value When such a range is expressed, another embodiment includes from the one particular value and/or to the other particular value. Similarly, when values are expressed as approximations, by the use of the antecedent ‘about,’ it will be understood that the particular value forms another embodiment.

Where a nucleic acid sequence is disclosed herein, the reverse complement thereof is also expressly contemplated.

Methods described herein may preferably be performed in vitro. The term ‘in vitro’ is intended to encompass procedures performed with cells in culture whereas the term ‘in vivo’ is intended to encompass procedures with/on intact multi-cellular organisms.

### BRIEF DESCRIPTION OF THE FIGURES

Embodiments and experiments illustrating the principles of the present disclosure will now be discussed with reference to the accompanying figures.

FIG. 1 293T cells KO HLA were transfected to express HaloTag -β2M-HLA-A *021. Cells stemming from a single clone generated by antibiotic selection and cells from the parental cell line were labeled with the HaloTag® ligand tetramethylrhodamine (HTL TMR). Saturation in HaloTag® ligand (HTL) staining was achieved at around 1 μM concentration and the same concentration resulted only in minor background signal increase in fluorescence on the parental cell line.

FIGS. 2A and 2B HaloTag®-β2M-HLA-A*02 01 293T cells were labeled with ssDNA-HTLs followed by a chase with the HTL AF488 (500 nM), ssDNA-HTLs reacted specifically with the HaloTag® (HT) and thus got attached to the cell surface as evident from the increase in fluorescence for the dye conjugated to the DNA as well as the decrease in the chase label. At around 10 μM of ssDNA-ITL the labeling achieved 60-70% completeness

FIG. 3: HaloTag®-D2M-HLA-A*02:01 293T cells were left unlabeled or w ere labeled with the HTL AF660 or the ssDNA-HT. Atto655. After pulsing the cells with a dilutions series of the NY-ESO-1 peptide. the cells served as target cells in a Jurkat activation assay. The Jurkats are modified to render the strength of TCR signaling into relative luminescence in this assay. The curves for the three conditions were close to one another and thus conjugation with the HTLs were not interfering with immune activation.

FIGS. 4A and 4B: HaloTag®-β2M-HLA-A*02:01 293T cells w ere labeled with the HTL TMR. After pulsing the cells with the WT-1 peptide for one hour at 37° C. in medium, they were used in co-culture with donor T cells and a bispecific antibody. The WT-1 bispecific antibody induces an artificial synapse by bridging CD3 on T cells and WT-1 pHLA on target cells. DP47 is a bispecific antibody with the exact same CD3 binder and identical layout but not recognizing a target in this assay. The co-cultures were set up with different target to effector cells ratios ranging from 5:1 to 1:5. After 2 hours the degrees of trogocytosis in those co-culture were analyzed by flow cytometry. The target and effector cells were distinguished by CD3 stain in the live, single cell population The median fluorescence intensity of TMR increased in the WT-1 bispecific antibody treated co-culture with a E:T 1:5 ratio to over 10 000. Lower E:T ratios, with thus less target cells, resulted in lower median fluorescence intensity (FT) (FIG. 4B), but still a high percentage of positive cells (FIG. 4A). The background noise overserved in the DP47 control was very low.

FIGS. 5A and 5B HaloTag®-β2M-HLA-A*02 01 293T cells were labeled with the HTL TMR. After pulsing the cells with the WT-1 peptide for one hour at 37° C. in medium, they were used in co-culture with donor T cells and a bispecific antibody. The WT-1 bispecific antibody induces an artificial synapse by bridging CD3 on T cells and WT-1 pHLA on target cells. DP47 is a bispecific antibody with the exact same CD3 binder and identical layout but not recognizing a target in this assay. The co-cultures were set up with different target to effector cells ratios ranging from 5:1 to 1:5. After 24 hours the degrees of trogocytosis in those co-culture were analyzed by flow cytometry. The target and effector cells were distinguished by CD3 stain in the live. single cell population The median fluorescence intensity of TMR increased in WT-1 bispecific antibody treated E:T 1 5 co-culture to over 10 000 Lower E:T ratios with thus less target cells resulted in low er median fluorescence intensity (F) (FIG. 513), but still a high percentage of positive cells (FIG. 5A). Compared to the to the 2 hour experiment, the percentage of positive cells had also increased in the co-culture with the E.T ratio of 5:1 from 40% to nearly 80%. The background noise overserved in the DP47 control was low, but a bit higher compared to the 2 hour experiment.

FIG. 6 HaloTag®-β2M-HL.A-A*0201 293T cells were labeled with the HTL TMR. After pulsing the cells with the MART-1 peptide for one hour at 37° C. in medium. they were co-cultured with a T cell pool having specificity for MART-L. The E.T was set to 1:1. After 1,2,3 and 24 hours the degrees of trogocytosis in those co-culture were analyzed by flow cytometry. The target and effector cells were distinguished by CD3 stain in the live, single cell population.

FIG. 7: The T cells of the co-culture where additionally analyzed for activation by looking at upregulation of CD69. An antibody stain revealed increased CD69 expression on T cells in pulsed co-cultures. The signal stayed elevated for the whole 24-hours' time window with low signal background in non-pulsed co-cultures.

FIG. : The T cells of the co-culture w ere additionally analyzed for activation by looking at upregulation of CD1 37. An antibody stain revealed increased CD1 37 expression on T cells in pulsed co-cultures at the 24 hours' time point. The signal in non-pulsed co-cultures stayed low for the whole 24-hour time window.

FIG. 9. The T cells of the co-culture were additionally analyzed for degranulation by looking at upregulation of CD107a An antibody stain revealed increased CD107a expression on T cells in pulsed co-cultures starting at the one-hour time point. The signal decreased over time and approached the background signal of T cell in non-pulsed co-cultures at the 24-hour time point.

FIGS. 10A and 10B: HaloTag®-2M-HL-A-A*02 01 293T cells were labeled with the HTL 15 AF660. After pulsing the cells with the NLV peptide for one hour at 37° C. in medium, they were co-cultured with a donor T cells of which around 1% have specificity for the CMV derived epitope NLV The E:T was set to 1:1 After 3 hours the degrees of trogocytosis in those co-culture were analyzed by flow cytometry. The target and effector cells were distinguished by CD3 stain in the live, single cell population. T cells with specificity for the NLV pH-ILA complex were revealed by dextramer stain (FIG. 10A) In all co-cultures (FIG. 10B), a percentage of the T cells became positive for the HaloTagged protein by trogocytosis. In pulsed co-cultures however, all T cells with specificity for the NLV epitope were also positive for the HTL.

FIG. 11. HaloTag -β2M-HLA*02:01 293T cells were labeled with the HTL-46 bp-Atto647N. After pulsing the cells with the MART-1 peptide for one hour at 37° C. in medium, they were co-cultured with a donor T cells of which around 1% have specificity for the tumor antigen MART-1. The E:T was set to 1 1. After 14 hours the degrees of trogocytosis in those co-culture were analyzed by flow cytometry The target and effector cells were distinguished by CD3 stain in the live, single cell population. T cells with specificity for the MART-1 pHLA complex were revealed by dextramer stain. The MFI derived from the barcode-HTL was higher on dextramer positive T cells in pulsed compared to non-pulsed co-cultures.

FIG. 12: T cells obtained from healthy donor subjects were fed with azide-modified sugars to install azide groups on their cell surfaces. to subsequently be used for strain-promoted alkyne-azide cycloaddition. After 48 hours. cells were incubated with a fluorescently-labeled peptide-DBCO conjugate for 120 minutes. Flow cytometry analysis of single. live cells showed specific attachment of the peptide to cells fed with azide-modified sugars, but not to control T cells., over the concentration range studied.

FIGS. 13A and 13B: Transduced Jurkat cells and T cells obtained from healthy donor subjects were fed with azide-modified sugars to install azide groups on their cell surfaces. to subsequently be used for strain-promoted alkyne-azide cycloaddition. After 48 hours, cells were incubated with a fluorescently-labeled peptide-DBCO conjugate and DOTAM-DBCO for 120 minutes, in different ratios Flow cytometry analysis of single, live cells showed high attachment of both components for a ratio of 10:1 peptide:DOTAM for T cells (A) and Jurkat cells (B).

FIGS. 14A and 14B: Transduced Jurkat cells were fed with azide-modified sugars to install azide groups on their cell surfaces, to subsequently be used for strain-promoted alkyne-azide cycloaddition. After 48 hours. cells were incubated with SortaseA acceptor peptide-DBCO conjugate and DOTAM_DBCO for 120 minutes. SortaseA was tethered to the effector cells using an antibody-enzyme conjugate directed against the hapten 293T HaloTag K-β2M-HL A-A*02.01 cells were labeled with the biotin-labeled sortase donor peptide-HT L conjugate, and pulsed to present the NY-ESO-1 antigen. Co-culture of the cells was found to result in peptide transfer after three (A) and 16 hours (B), as determined by an increase of biotin staining on the single, live effector cells.

FIG. 15 Transduced Jurkat cells were fed with azide-modified sugars to install azide groups on their cell surfaces, to subsequently be used for strain-promoted alksne-azide cycloaddition. After 48 hours, cells were incubated with SortaseA acceptor peptide-DBCO conjugate and DOTAM DBCO for 120 minutes. SortaseA % as tethered to the effector cells using an antibody-enzyme conjugate directed against the hapten. 293T HaloTag®-2M-HLA-A*02:01 cells were labeled with the biotin-labeled ssDNA peptide-HTL conjugate, and pulsed to present the NY-ESO-1 antigen. Co-culture of these cells was found to result in peptide transfer after 16 hours, as determined by an increase of biotin staining on the single, live effector cells.

### EXAMPLES

### Example 1: Materials and Methods

### 1.1 Recombinant DNA Techniques

Standard methods were used to manipulate DNA as described in Sambrook el al, Molecular Cloning: A Laboratory Manual Cold Spring Harbor Laboratory Press, Cold Spring Harbor. New York. 1989. The molecular biological reagents were used according to the manufacturers' instructions.

1.2 DNA sequencing

DNA sequences were determined by double strand Sanger sequencing.

### 1.3 Gene Synthesis

Desired gene segments where required were synthesized by Genscript Biotech (New Jersey, US) from synthetic oligonucleotides and PCR products by automated gene synthesis The gene segments flanked by single restriction endonuclease cleavage sites were cloned into standard cloning/sequencing vectors. The plasmid DNA %% as purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors.

### 1.4 Generation of Halo-Tagged Cell Lines

To produce a cell line expressing an HLA-A*02:01 comprising a HaloTag® fusion at the cell surface, a genetic fusion of the HaloTag® k enzyme to human β2M joined by a linker to HLA-A*02.01i was generated by standard cloning techniques, and sub-cloned into a suitable expression vector. The amino acid sequences of the nascent and mature HaloTag®-β2M-HLA-A02 fusion protein are shown in SEQ ID NOs:12 and 13.

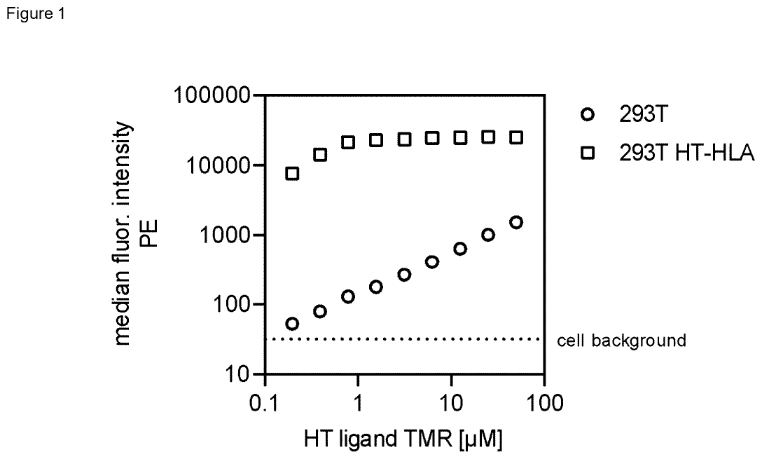

The plasmid encoding the HaloTag®-β2M-I-HLA-A02 fusion protein was introduced by lipofection into 293T cells modified by CRIPSR to knockout endogenous HLA loci. Transfected clones were selected by standard antibiotic selection. Resulting clones were further selected 30 based on a positive antibody stain for HLA-A*02:01 and 132M as determined by analysis by flow cytometry Selected positive clones were eventually exposed to a dilution series of the HaloTag® ligand (HTL) with the fluorophore tetramethylrhodamine (TMR): HTL TMR). Briefly. cells were detached. washed with PBS and incubated with HTL TMR at a density of 1×10⁶ cells/nil in PBS for 30 minutes. Cells were then washed with PBS and stained with a standard live/dead stain before being analyzed by flow cytometry Events were gated based on forward- and side-scatter, followed by exclusion of doublets and dead cells. The cellular median fluorescence intensity (MF1) was plotted against the concentration of the HIT. From a concentration of 1 μM, complete labeling was achieved for the final clone (FIG. 1). Low background labeling on the parental cell line was seen at 1 μM.

1.5 Attaching DNA barcodes to cells via the HaloTag® system

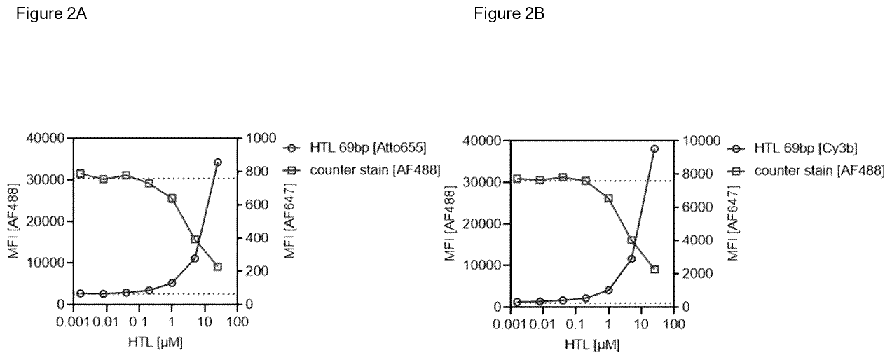

DNA oligonucleotide barcodes to be employed with the 29′3T HaloTag:-02M-HLA-A*02:01 cell line were prepared as follows. The 3′ end of 69 bp oligos having a nucleotide sequence conforming to the consensus shown in SEQ ID NO. 8 I were modified with a fluorophore (Atto655 or Cy 3b), and the 5′ end was conjugated to the HTL. The modified oligos were obtained from Biomers GmbH, and are hereafter referred to as ‘DNA-HTL’ 293T HaloTag®-(2M-HLA-A*02 01 cells were detached, washed with PBS and incubated with a dilution series of the DNA-HTL in modified DPBS (Gibco. #14287080) at a cell density of I x 10⁶ cells/ml for 120 minutes. Cells were then washed with PBS and stained with a standard live/dead stain. Furthermore, a HTL AF488 chase was included at 500 nM in PBS for 5 minutes Cells were subsequently analyzed by flow cytometry. Events were gated based on forward- and side-scatter. followed by exclusion of doublets and dead cells. The median fluorescence intensity (MFI) of the DNA-HTL and the chase were plotted against the concentration of the DNA-HTL. From a concentration of 10 μM labeling of about 60-70% was achieved (FIG. 2)

### 1.6 Preparation of Virus Like Particles (VLPs)

Lipofectamine® LTX-based transfection was performed using ˜70% confluent Lenti-X™293T cells (Takara, #632180) and the construct encoding transfer vectors as well as packaging vectors pCAG-VSVG and psPAX2 at a 2:1:2 molar ratio (Girn-Laterriere M. er at. Methods Mol Biol. 2011:737:183-209, Myburgh R. et al Mol Ther Nucleic Acids. 2014). As control for every experiment. mock virus-like particles (VLPs) using only the packaging vectors, but no transfer vector, were produced After 48 hours. the supernatant was collected and remaining cells were removed by centrifugation. VLPs were used directly or stored at −80° C.

1.7 Transduction of Jurkat cells

1×10⁶ Jurkat cells per well were seeded in wells of a 24-well plate. V LPs were used fresh or thawed at 37° C., and 300 pl were added together with 8 μg/ml Polybrene (Sigma Aldrich) and Lentiboost P (1:100) (Sirion Biotech, #SB-P-LV-101-12) to a 24 well plate for spinfection at 1100 x g for 99 min and 31° C. The cells were incubated for at least 72 hours at 37 ‘C, 5% CO₂ before the transduction was evaluated by flow cytometry.

1.8 Isolation of Primary T Cells from Blood

Donor blood was sourced from Blutspende Zuirich (Rutistrasse 19, 8952 Schlieren). Leucosep™ tubes (Fisher Scientific, #10349081) with 15 mL of room temperature Histopaque density gradient medium (Sigma-Aldrich. #10771) were prepared and centrifuged at 400× g for 5 minutes, until the Histopaque had passed the filter The blood was diluted with an equal amount of PBS. 30 ml of the blood/buffer mixture was added to the Leucosep” tubes. Tubes were centrifuged 1200× g for 20 min with the breaks off The band containing the peripheral blood mononuclear cells (PBMCs) was carefully pipetted into a fresh 50 ml Falcon tube and topped-up to 50 ml with DPBS. The cells were washed 3 times with DPBS and finally resuspended in DPBS and counted. Pan T cell isolation was performed by negative selection using the Pan T cell isolation kit (Miltenyi, #130-096-535), in accordance with the manufacturer's instructions. The cells were either frozen or used directly after isolation. Cells were cultured in advanced RPMI (Gibco, #1 1530446). 10% FBS (Sigma, #F4135-500M1.). 1% GlutaMAX™ (Gibco. #35050-038). 50 IU/PROLEUKINt (recombinant IL-2: Novartis), 25 ng/ml IL-7 (Miltenvi, #130-095-364) and 50 ng/ml IL-15 (Miltenyi, #130-095-766),

1.9 Metabolic glyco-engineering of cells and strain promoted alkyne-azide cycloaddition of peptides and small molecules

Cells w ere cultured in standard cell culture medium containing 50 μM N-azidoacetyimannosamine tetraacslated (ManNAz) for 24-48 hours to install click handles on the surface of the cells. The functionalized cells were incubated with DOTAM-DBCO and/or peptide conjugates having the following structures: [6-FAMI-GG(GG-[CYS(DBCO-MAL)f or ‘GGGGG-ICYS(DBCO-MAL)[‘for two hours in PBS, with different concentrations and ratios of the components.

1.10 Production of IgG-Like Proteins in Expi293™ Cells Antibodies and bispecific antibodies were generated by transient transfection of Expi293™ cells. Cells

were seeded in Expi293™ media (Gibco, #1435101) at a density of 2 5× 10⁶/ml. Expression vectors and ExpiFectamine™ (Gibco, ExpiFectamine™ transfection kit, #13385544) were separately mixed in OptiMEM™ (Gibco, #11520386). After 5 min, both solutions were combined, mixed by pipetting and incubated for 25 min at room temperature. Cells were added to the vector/ExpiFectamine™ solution and incubated for 24 hours at 37° C. in a shaking incubator with a 5% CO₂ atmosphere. One day post-transfection, supplements (Enhancer 1+2, ExpiFectamine™ transfection kit) were added. Cell supematants were harvested after 4-5 days by centrifugation and subsequent filtration (0.2 pm filter), and proteins were purified from the harvested supernatant by standard methods as indicated below

### 1.11 Purification of IgG-Like Proteins

Proteins were purified from filtered cell culture supernatants. Briefly. proteins were purified from cell culture supematants by Protein A or kappa-select affinity chromatography (equilibration buffer: 20 mM sodium citrate, 20 mM sodium phosphate, pH 7.5; elution buffer: 20 mM sodium citrate, pH 3.0). Elution was achieved at pH- 3.0 followed by immediate pH neutralization of the sample. The protein was concentrated by centrifugation (Millipore Amicon® ULTRA-15. #UFC903096), and aggregated protein was separated from monomeric protein by size-exclusion chromatography in 20 mM histidine. 140 mM sodium chloride, pH 6 0.

### 1.12 Analytics of IgG-Like Proteins

The concentrations of purified proteins were determined by measuring the absorbance at 280 nm using the mass extinction coefficient, calculated on the basis of the amino acid sequence according to Pace et al., Protein Science (1995) 4: 2411-1423. The purity and molecular weight of the proteins was analyzed by CE-SDS in the presence and absence of a reducing agent, using a LabChipX GXII or LabChipK GX11 Touch™ (Perkin Elmer) Determination of the aggregate content was performed by HPLC chromatography at 25° C. using analytical size-exclusion column (TSKgel G3000 SW XL or UP-SW3000) equilibrated in running buffer (200 mM KH₂PO₄, 250 mM KCl pH 6.2, 0 02% NaN3)

1.13 Coupling of IgGs with Fluorescent Dyes

Purified IgGs were labeled with fluorophores for subsequent use for analysis by flow cytometry Briefly, an appropriate amount of protein was labeled using commercially-available kits such as Alexa Fluor™ 647 Antibody Labeling Kit #A20186 (ThermoFisher). The resulting conjugates were analyzed in order to determine the degree of labelling, and the ideal amount of antibody to use for staining procedures was determined individually in titration experiments.

1.14 Peptide Barcodes to Use with the HaloTag® System

A peptide having the structure ‘[Biotin-Ahx]-SELPETGK’ was conjugated to the HTL via an ester reaction. The resulting conjugates are hereafter referred to as -peptide HTL. 293T HaloTag®-β2M-HL A-A*02.01 cells were detached. washed with PBS and incubated with 100 μM of peptide HTL in PBS, at a cell density of 3×10⁶ cells/mi, for 120 minutes. Cells were then washed with PBS and used in co-cultures.

1.15 DNA-Peptide Barcodes to Use with the HaloTag System

DNA oligonucleotide peptide barcodes to be employed with the 293T HaloTag®-β2M-HLA-A*02.01 cell line were prepared as follows. The 3′ end of 69 bp oligos having a nucleotide sequence confining to the consensus shown in SEQ ID NO. 18 were modified with ‘Biotin-TEG’, and the 5′ end was modified with ‘DBCO-TEG’. The modified oligos were obtained from IDT as HPLC-purified products. A peptide having the structure ‘[Lys(N3)|-SELPETGK’ was conjugated to the HTL via an ester reaction, and conjugated to the oligo via strain promoted alkyne-azide cycloaddition. The resulting conjugates are hereafter referred to as ‘DNA-peptide HFTL’, and have the following structure : Biotin-DNA-I DBCO/Azidel-peptide-[Amine/Ester]-HTL, wherein ‘[DBCO/Azide]’ indicates the connection formed by a strain-promoted alkyne-azide cycloaddition reaction between DBCO and an azide moiety, and wherein ‘[Amine/Ester]‘indicates ester bond formed by dehalogenase activity of the HaloTag® on a HaloTag® ligand comprising a chloroalkane moiety. 293T HaloTag®-β2M-HLA-A*02:01 cells were detached. washed with PBS and incubated with 100 μM if DNA-peptide HTL in modified DPBS (Gibco, #14287080), at a cell density of 3×10⁶ cells/mi, for 120 minutes. Cells were then washed with modified DPBS and used in co-cultures.

### Example 2: Conjugation of Halotag Lip-Ands does not Interfere with Activation of Immune Cells

The inventors investigated whether the proximity of the conjugated HTLs of the 293T HaloTag®-β2M-1LA-A*02 01 cell line to the TCRs of immune cells interferes with proper synapse formation and activation of the immune cells. Possible steric and charge-based interference was evaluated in a Jurkat activation assay. A Jurkat parental cell line modified to express a luciferase under the control of TCR-signaling- inducible promoter, having had their TCR deleted. endogenously expressing CD4, and also engineered to express CD8a was obtained from Promega (#GA 1162). The cells were additionally modified to present the 1G₄ TCR specific for NY-ESO-I by lentiviral transduction. The supplied protocol was followed for the assay. Briefly. 293T HaloTag®-β2M-HLA cells were detached, washed with PBS and labeled at optimal conditions as described above with HTL AF660 and a HTL-ssDNA-(69 bp)-Atto655 The labeled cells were pulsed with a dilution series of the NY-ESO-1 antigen peptide ‘SLLMWITQC’(SEQ ID NO: 14) for one hour at 37° C. in growth medium. Thereafter, a co-culture of the labeled and pulsed cells with the Jurkat cell line was performed at a ratio of 1:1 and I x 10′ cells per well. The assay was performed in a 96-well white F-bottom chimney plate.

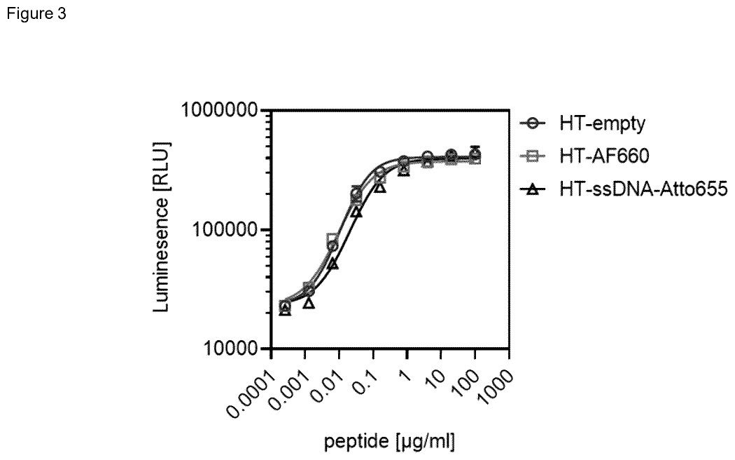

After 7 hours. the provided substrate (Promega. #J3081) was added and the resulting luminescence was read at a Tecan Spark multimode microplate reader. Plotting the peptide concentration vs. luminesce and fitting concentration-response curves using GraphPad Prism version 8 revealed no major difference of fluorophore or DNA-conjugated cells compared to cells with empty HaloTag® (FIG. 3),

### Example 3: TroGocytosis of HaloTagged HLA induced by Synthetic Immunity

Bispecific antibodies engaging CD3 on T cells and a target antigen on tumor cells can be used to engage and activate T cells, and direct their effector activity against cells expressing the target antigen In the artificial synapse. material is transferred from the engaged target cell onto the CD3-TCR complex-expressing cell by trogocytosis. A bispecific antibody recognizing WT-I (Augsberger el al., Blood (2021) 138(25).2655-2669) was used to evaluate Mether HaloTagged-HLA molecules can be transferred in this way.

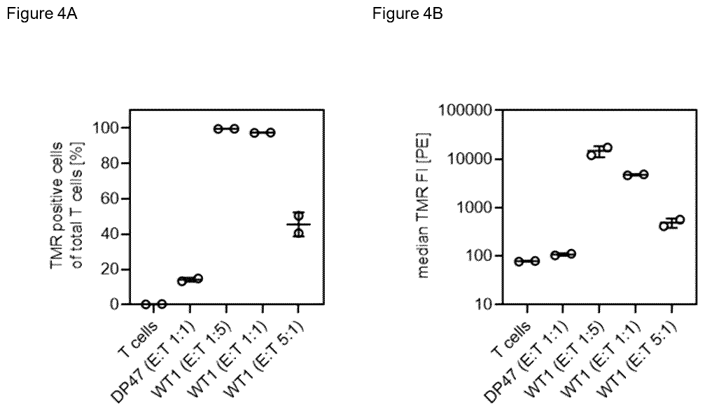

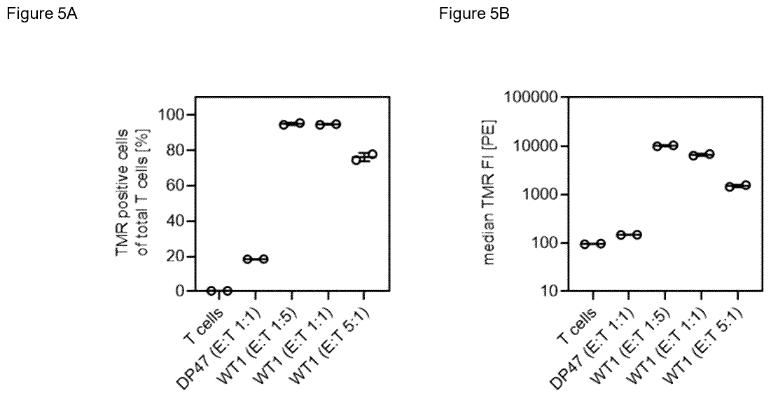

Human T cells were isolated from donor blood by standard procedure. 293T HaloTag) -02M-HLA-A*02l cells were labeled with I-TL TMR (as described above) and pulsed with the WT-I antigen peptide ‘RMFPNAPYI’ (SEQ ID NO: 15), which forms the epitope of the WT-1-binding arm of the bispecific antibody (described above). Next, a co-culture of effector and WT-1 peptide-pulsed target cells with the bispecific antibody was set-up in technical duplicates. The concentration of the bispecific antibody was fixed to 10 μg/ml. while the effector-to-target cell ratio varied from 5:1. 1:1 and 1:5 with a constant total cell count of 5×10′ cells per well in 100 μl of medium. The resulting trogocytosis was evaluated at 2 and 24 hours later by flow cytometry. From the live. single cell population, target and effector cells were distinguished by CD3 stain. Effector cell median fluorescence intensity in the TMR channel and the percentage of cells being positive for TMR of total effector cells were exported and further analyzed. The WT-1-targeting bispecific antibody induced trogocytosis of FHaloTag®-J12M-HLA-A*02:0II from target-to-effector cells already after two hours (FIGS. 4A and 4B) and was also seen after 24 hours (Figures SA and 5B). Very low background staining was observable with the non-target control bispecific antibody DP47 after two (FIGS. 4A and 4B) and 24 hours (FIGS. 5A and 5B).

### Example 4: Trogocytosis of HaloTagged H1LA Induced by TCR Interaction in a Pool with Given Specificity

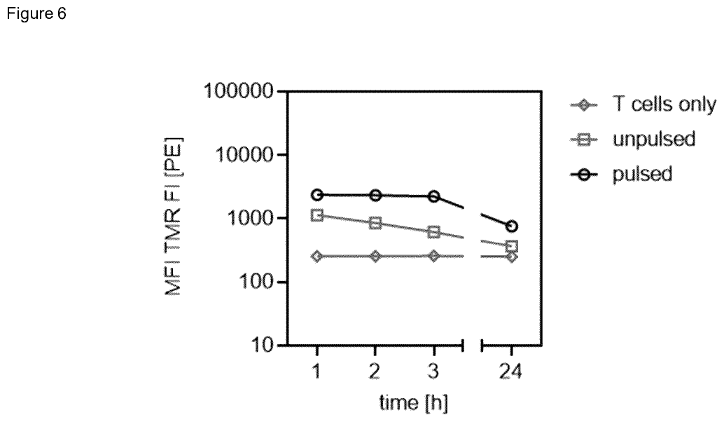

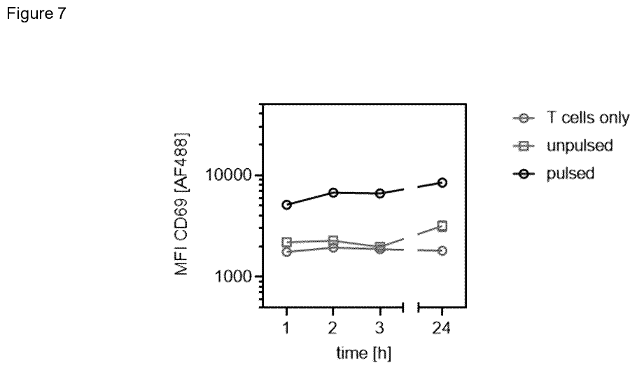

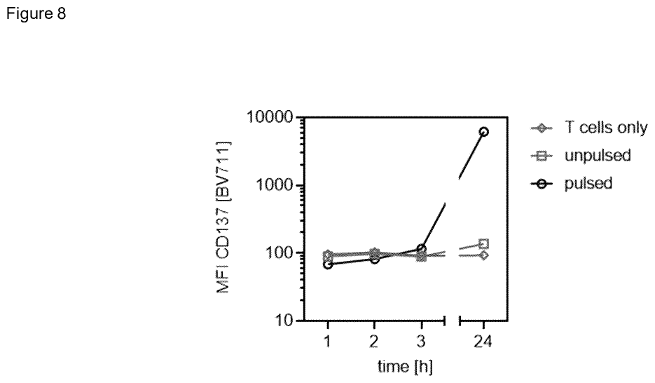

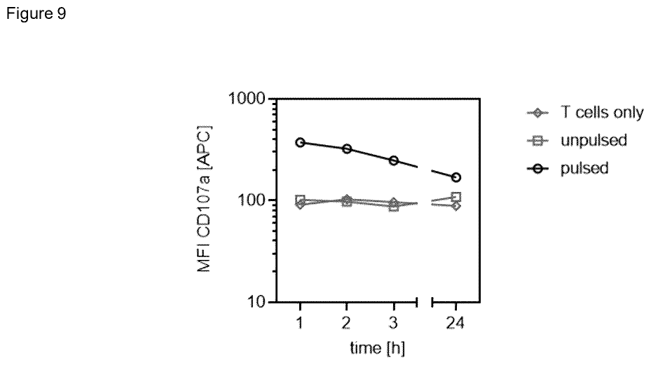

To test if HaloTagged HLA can also be transferred by TCR-driven trogocytosis, a polyclonal pool of T cells with specificity for the twnor-associated target MART-1 was used. 293T HaloTag®-β2M-1LA-A*02 O1 cells were labeled with HTL TMR (as described above) and pulsed with the MART-I antigen peptide -ELAGIGILTV’ (SEQ ID NO:16). Next, a co-culture of effector and target cells with a 1: 1 ratio and 5×10⁵ cells per w ell was set up. The resulting trogocytosis was analyzed 1, 2. 3 and 24 hours later by flow cytometry. From the live, single cell population, target and effector cells were distinguished by CD3 stain. Effector cell median fluorescence intensity in the TMR channel and percentage of cells being positive for TMR of total effector cells were exported and further analyzed as well as median fluorescence intensity of CD69, CD107a and CD137 T cells indeed became positive for the HaloTagged molecule as early as 1 hour after start of the co-culture (FIG. 6). The MFI was consistently higher compared to non-pulsed control cells over the whole period. Additionally. T cells were activated as seen by CD69 upregulation early on (FIG. 7) and CD137 after 24 hours (FIG. 8). Eventually. T cells showed degranulation as evident from CD107a appearing on the cell surface (FIG. 9)

### Example 5: Trogocytosis of HaloTagged HLA Induced by TCR Interaction Using Donor T Cells with More than One Specificity

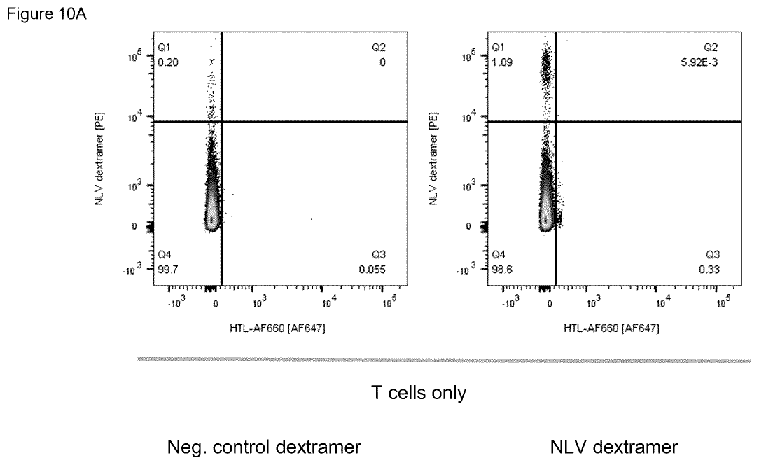

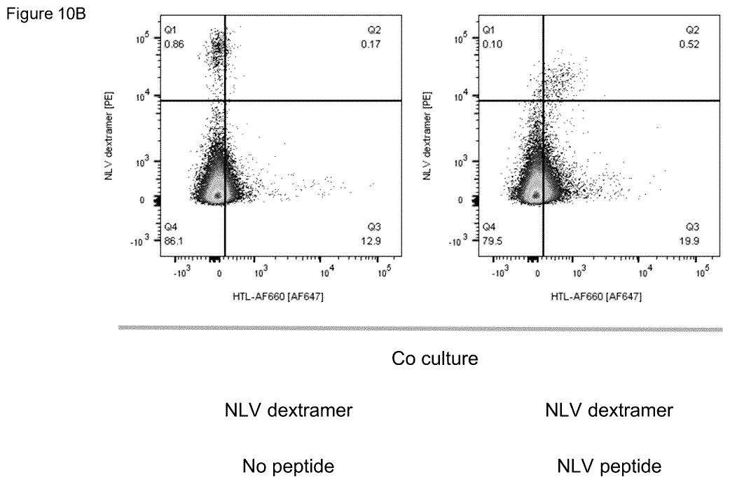

To test if HaloTagged HLA can also be transferred onto T cells having a given specificity present at low frequency within a pool of T cells having diverse specificities. donor-derived T cells were screened for the presence of HLA-A*02:01-restricted antiviral T cells bs dextramer stain (Immudex, Denmark). These donor T cells were used for the co-culture. 293T HaloTag®-D2M-HLA-A*02:01 cells were labeled with HTL AF660 (as described above) and pulsed with the CMV antigen peptide ‘NLVPMVATV’ (SEQ ID NO:17). Next, a co-culture of effector and target cells with a 1 1 ratio and 3×10⁵ cells per well was set up. The resulting trogocytosis was analyzed three hours later by flow cytometry From the live, single cell population. target and effector cells were distinguished by CD3 stain. Cells with the NLV specificity were identified by dextramer staining (Immudex. Denmark). While a certain fraction of T cells became positive for the HaloTag® independent of peptide pulsing, all NLV-reactive cells were HaloTag, positive in peptide-pulsed co-cultures (FIGS. 10A and 10B.

### Example 6: Trogocytosis of ssDNA Barcodes Attached to HaloTagged HLA Induced by TCR Interaction Using Donor T Cells with More than One Specificity

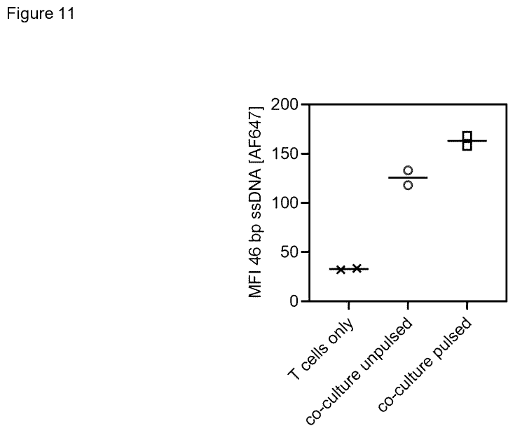

To evaluated whether DNA conjugated to the HaloTag®-β2M-HLA-A*02:M molecules can also be transferred onto present at low frequency within a pool of T cells having diverse specificities, donor-derived T cells were screened for the presence of 1L1.A-A*O2 1-restricted tumor-associated T cell epitopes such as MART-1 by dextramer stain (Immudex. Denmark). These donor T cells were used for the co-culture. 293T HaloTag®-R2M-HLA-A*02:0l cells were labeled with HTL 46 bp Expi293™ (as described above) and pulsed with the MART-1 peptide (SEQ ID NO 6). Next, a co-culture of effector and target cells with a 1:1 ratio and 3×10⁵ cells 10 per well was set up. The resulting trogocytosis was analyzed 14 hours later by flow cytometry. From the live, single cell population, target and effector cells were distinguished by CD3 stain. Cells with the MART-1 specificity were identified by dextramer staining (Immudex. Denmark). Pulsed cultures had a higher MFI in the Atto647N channel in the MART-1 positive population compared to non-pulsed controls (FIG. 11)

### Example 7: Functionalization of Glyco-Engineered Cells to Attach Peptides to the Surfaces of the Cells

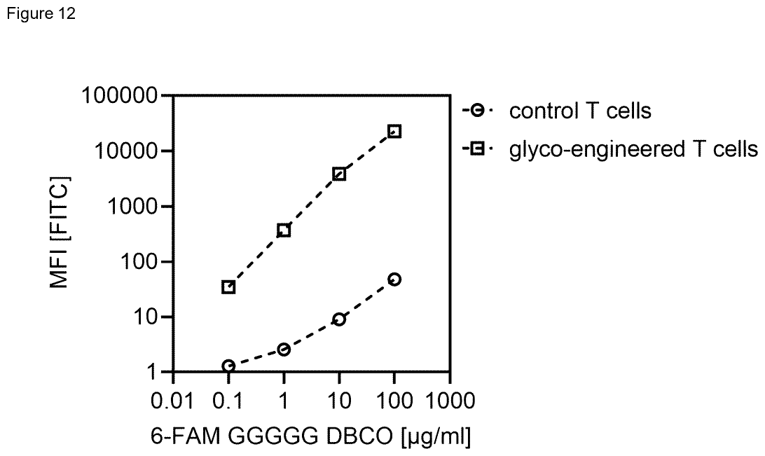

Healthy, donor T cells were functionalized with peptides attached to glycoproteins as follows. Cells were cultured for 48 hours in the presence of azide-modified sugars. As a consequence, azide-moieties were presented on the surface of the cells, and thus available for strain promoted alkyne-azide cycloaddition. Cells were incubated for two hours with different concentrations of a fluorescently-labeled, peptide-DBCO conjugate. Washed cells were subsequently analyzed by flow cytometry. Analysis of single. live cells revealed specific and concentration-dependent attachment of peptides, compared to not-engineered control cells (FIG. 12),

### Example 8: Functionalization of Glyco-Engineered Cells to Attach Small Molecules and Peptides to the Surfaces of the Cells

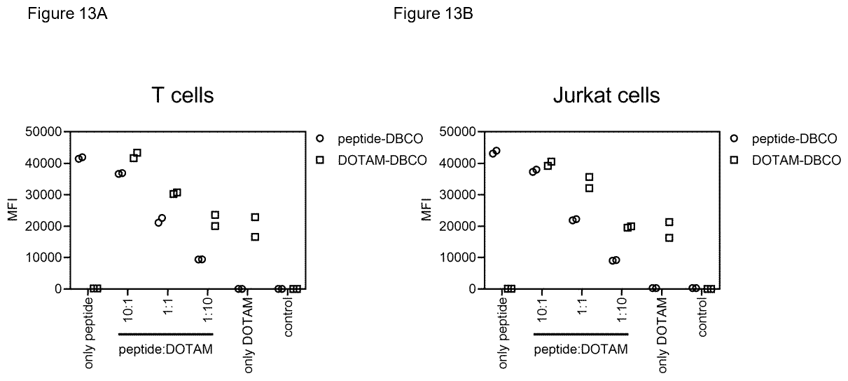

Transduced Jurkat cells and healthy donor T cells were functionalized with peptides and small molecules attached to glycoproteins as follows. Cells were cultured for 48 hours in the presence 30 of azide-modified sugars. As a consequence, azide-moieties were presented on the surface of the cells, and thus available for strain promoted alkyne-azide cycloaddition Cells were incubated for two hours with different ratios of a fluorescently-labeled. peptide-DBCO conjugate and/or DOTAM-DBCO. with total concentration fixed to 10 μg/ml. DOTAM conjugation was detected using a fluorescently -labeled DOTAM-specific antibody (the antibody formed of polypeptides having the amino acid sequences of SEQ ID NOs 29 and 31. labeled with AF647) Washed cells were subsequently analyzed by flow cytometry Analysis of single, live cells revealed specific and ratio-dependent attachment of the peptide and the bapten (FIG. 13),

### Example 9: Transferring a Peptide Between Cells by Tethered sortaseA in Co-Cultures

Transduced Jurkat cells were functionalized with peptides and DOTAM was attached to glycoproteins as described in Example 8 A modified version of sortase A was subsequently was tethered to the cells through the using an anti-DOTAM antibody-sortase A conjugate (formed of polypeptides having the amino acid sequences of SEQ ID NOs: 30 and 32),

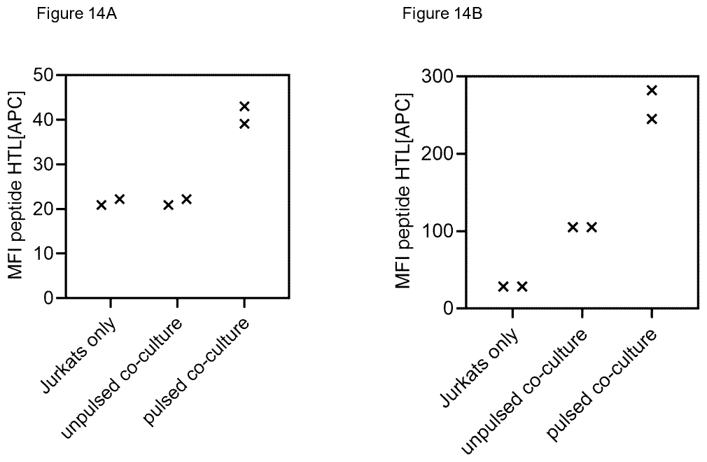

293T HaloTag.-02M-HL A-A*(2:01 cells were labeled with the peptide HTL (as described in Example 1.14) and pulsed with the NY-ESO-1 antigen peptide ‘SLLMWITQC’ (SEQ ID NO. 14). Next, a co-culture of effector and target cells with a 1.1 ratio and 3×10³ cells per well was set up. The resulting peptide transfer was analyzed three and sixteen hours later by flow cytometry. Within the population of live, single cells, target and effector cells were distinguished by CD45 staining. A specific increase in peptide HTL on effector cells was detected by anti-biotin antibody staining, and was observed after three (FIG. 14A) and sixteen hours of co-culture (FIG. 14B),

### Example 10: Transferring a Barcode Construct Between Cells by Tethered sortaseA in Co-Cultures

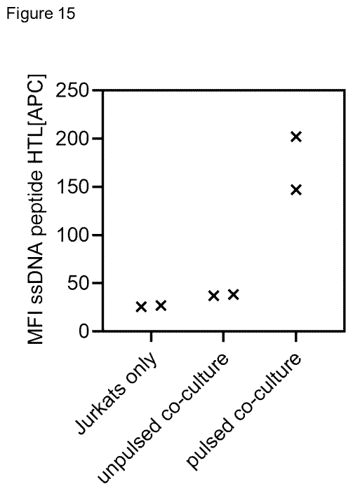

Transduced Jurkat cells were functionalized with peptides and DOTAM was attached to glycoproteins as described in Example 8. A modified version of sortase A was subsequently was tethered to the cells through the use of an ani-DOTAM antibody -sortase A conjugate (formed of polypeptides having the amino acid sequences of SEQ ID NOs 30 and 32), 293T HaloTag.-02M-HL A-A*(2:01 cells were labeled with the DNA-peptide HTL (as described in Example 1 15) and pulsed with the NY-ESO-1 antigen peptide ‘SLtLMWITQC’ (SEQ ID NO. 14) Next, a co-culture of effector and target cells with a 1:1 ratio and 3×10⁵ cells per well was set up. The resulting peptide transfer w as analyzed sixteen hours later by flow cytometry. Within the population of live. single cells, target and effector cells were distinguished by CD45 staining. A specific increase in DNA-peptide HTL on effector cells was detected by anti-biotin antibody staining (FIG. 15).

The present disclosure relates to the fields of molecular biology and immunology.

1. A polypeptide comprising (i) the amino acid sequence of a major histocompatibility complex (MHC) polypeptide, and (ii) a moiety facilitating labelling of the polypeptide with an identifier moiety.

2. The polypeptide according to claim 1, wherein the MHC polypeptide is β2 microglobulin.

3. The polypeptide according to claim 1 or claim 2, wherein the identifier moiety is a nucleic acid moiety, optionally wherein the identifier moiety comprises or consists of single-stranded DNA (ssDNA),

4. The polypeptide according to any one of claims 1 to 3, wherein the moiety facilitating labelling of the polypeptide with an identifier moiety is or comprises a self-labelling protein tag.

5. The polypeptide according to any one of claims 1 to 4, wherein the moiety facilitating labelling of the 15 polypeptide with an identifier moiety is or comprises a HaloTag.

6. The polypeptide according to any one of claims 1 to 5, wherein the polypeptide further comprises a sortase substrate motif.

7. The polypeptide according to any one of claims 1 to 6, further comprising a detectable moiety.

8. The polypeptide according to claim 7, wherein the detectable moiety is a fluorescent label.

9. The polypeptide according to any one of claims 1 to 8, wherein the polypeptide comprises or consists of an amino acid sequence having at least 70% amino acid sequence identity to SEQ ID NO:11, 10, 13 or 12.

10. A polypeptide comprising (i) the amino acid sequence of β2 microglobulin, and (ii) HaloTag.

11. A polypeptide comprising (i) the amino acid sequence of β2 microglobulin, (ii) a sortase substrate motif, and (iii) HaloTag.

12. An MHC molecule comprising a polypeptide according to any one of claims 1 to 11.

13. A MHC:peptide complex, comprising a MHC molecule according to claim 12, and a peptide presented by the MHC molecule.

14. A nucleic acid, or a plurality of nucleic acids, encoding a polypeptide according to any one of claims 1 to 13.

15. The nucleic acid or plurality of nucleic acids according to claim 14, further comprising nucleic acid encoding a peptide presented by an MHC molecule comprising a polypeptide according to any one of paras 1 to 11.

16. An expression vector, or a plurality of expression vectors, comprising a nucleic acid, or a plurality of nucleic acids, according to claims 14 or 15.

17. A cell comprising a polypeptide according to any one of claims 1 to 11, an MHC molecule according to claim 12, a MHC:peptide complex according to claim 13, a nucleic acid or plurality of nucleic acids according to claim 14 or 15, or an expression vector or plurality of expression vectors according to claim 16.

18. The cell according to claim 17, wherein the cell is an antigen presenting cell (APC),

19. A method for producing a cell comprising an MHC molecule labelled with an identifier moiety, comprising:
(1) introducing into a cell a nucleic acid or plurality of nucleic acids according to claim 15; and
(2) contacting the cell with a labelling moiety comprising an identifier moiety, wherein the labelling moiety is suitable for labelling the polypeptide encoded by the nucleic acid or plurality of nucleic acids of (1) with an identifier moiety.

20. A method for producing a cell comprising an MHC:peptide complex comprising an MHC molecule labelled with a single-stranded DNA (ssDNA) moiety, comprising:
(1) introducing into a cell a nucleic acid or plurality of nucleic acids encoding a polypeptide comprising (i) the amino acid sequence of β2 microglobulin, and (ii) a HaloTag;
(2) introducing into the cell: (i) a peptide presented by an MHC molecule comprising the polypeptide of (1), or (ii) nucleic acid encoding a peptide presented by an MHC molecule comprising the polypeptide of (1); and
(3) contacting the cell with a HaloTag ligand comprising the ssDNA moiety and a choroakane moiety.

21. A method for producing a cell comprising a MHC:peptide complex comprising a MHC molecule labelled with a single-stranded DNA (ssDNA) moiety, comprising:
(1) introducing into a cell a nucleic acid or plurality of nucleic acids encoding a polypeptide comprising (i) the amino acid sequence of β2 microglobulin, (ii) a sortase substrate motif, and (iii) a HaloTag;
(2) introducing into the cell: (i) a peptide presented by a MHC molecule comprising the polypeptide of (1), or (ii) nucleic acid encoding a peptide presented by a MHC molecule comprising the polypeptide of (1); and
(3) contacting the cell with a HaloTag ligand comprising the ssDNA moiety and a choroakane moiety.

22. A cell produced by the method according to any one of claims 19 to 21.

23. A composition comprising a cell according to any one of claims 17, 18 or 22, and a T cell.

24. A method for identifying a T cell receptor (TCR) that binds to an MHC:peptide complex, comprising:
(1) contacting a cell comprising an MHC:peptide complex according to claim 13 with a population of T cells;
(2) incubating the cells under conditions suitable for trogocytosis of an MHC:peptide complex by a T cell comprising a TCR that binds to the MHC:peptide complex; and
(3) subsequently analysing T cells to identify a TCR that binds to the MHC:peptide complex.

25. A method for identifying a T cell receptor (TCR) that binds to a MHC:peptide complex, comprising:
(1) contacting (a) a cell comprising a MHC:peptide complex with (b) a population of T cells;
(2) incubating the cells obtained after step (1) under conditions suitable for trogocytosis of a MHC:peptide complex by a T cell comprising a TCR that binds to the MHC:peptide complex; and
(3) subsequently analysing the cells obtained after step (2) to identify a TCR that binds to the MHC:peptide complex;
wherein the MHC:peptide complex comprises a MHC molecule, and wherein the MHC molecule comprises a polypeptide comprising (i) the amino acid sequence of a MHC polypeptide, and (ii) an identifier moiety, wherein the identifier moiety is covalently associated with the polypeptide via linkage formed by a self-labelling protein tag.

26. A method for identifying a T cell receptor (TCR) that binds to a MHC:peptide complex, comprising:
(1) contacting (a) a cell comprising a MHC:peptide complex with (b) a population of cells comprising T cells comprising a polypeptide comprising a sortase acceptor motif at the cell surface, in the presence of a sortase;
(2) incubating the cells obtained after step (1) under conditions suitable for interaction between the cell of (α) and the population of cells of (b); and
(3) subsequently analysing the cells obtained after step (2) to identify a T cell comprising a TCR that binds to the MHC:peptide complex;
wherein the MHC:peptide complex comprises a MHC molecule, and wherein the MHC molecule comprises a polypeptide comprising (i) the amino acid sequence of a MHC polypeptide, (ii) a sortase substrate motif, and (iii) an identifier moiety, wherein the identifier moiety is covalently associated with the polypeptide via linkage formed by a self-labelling protein tag.

27. The method according to claim 26, wherein the sortase is provided at the cell surface of T cells comprising a polypeptide comprising a sortase acceptor motif.

28. The method according to any one of claims 25 to 27, wherein the MHC polypeptide is β2 microglobulin.

29. The method according to any one of claims 25 to 28, wherein the identifier moiety is a nucleic acid moiety, optionally wherein the identifier moiety comprises or consists of single-stranded DNA (ssDNA).

30. The method according to any one of claims 25 to 29, wherein the self-labelling protein tag is or comprises a HaloTag.

31. The method according to claim 30, wherein the identifier moiety is covalently associated with the polypeptide via an ester bond formed by dehalogenase activity of the HaloTag on a HaloTag ligand comprising the identifier moiety and a chloroakane moiety.
